(19) United States
(12) Patent Application Publication
(10) Pub. No.: US 2021/0348124 A1
(43) Pub. Date: Nov. 11, 2021

(54) SEBACEOUS GLAND ORGANOIDS AND USE THEREOF

(71) Applicant: TECHNION RESEARCH & DEVELOPMENT FOUNDATION LIMITED, Haifa (IL)

(72) Inventors: Yaron FUCHS, Haifa (IL); Alona FELDMAN, Halutz (IL)

(21) Appl. No.: 17/276,770

(22) PCT Filed: Sep. 16, 2019
(86) PCT No.: PCT/IL2019/051034

§ 371 Date: Mar. 16, 2021

(30) Foreign Application Priority Data
Sep. 17, 2018 (IL) 261849

(51) Int. Cl. C12N 5/071; A61K 35/36

(52) U.S. Cl. CPC C12N 5/0633; A61K 35/36; C12N 5/063; C12N 2513/00; C12N 2500/30; C12N 2503/02; C12N 2501/11; C12N 2501/119; C12N 2500/14; C12N 2533/90; C12N 2501/999

(57) ABSTRACT

Sebaceous gland-like organoids comprising a Blimp1 positive cell are provided. Methods of producing the organoids and using the organoids to test a skin drug are also provided; as are methods of treating acne.

### CROSS REFERENCE TO RELATED APPLICATIONS

This application claims the benefit of priority to Israel Patent Application No. 261849 filed Sep. 17, 2018, entitled “SEBACEOUS GLAND ORGANOIDS AND USE THEREOF” the contents of which are incorporated herein by reference in their entirety.

### FIELD OF INVENTION

The present invention is in the field of organ modeling and drug testing.

### BACKGROUND OF THE INVENTION

The epidermis consists of three main compartments: the hair follicle (HF), interfollicular epidermis (IFE) and the sebaceous gland (SG). While the HF and IFE have been the focus of numerous investigations, incredibly little is known regarding the homeostasis of the SG and mechanisms regulating this unique mini-organ.

The mouse HF cycles between phases of growth (anagen), destruction (catagen) and rest (telogen) and is fueled by subpopulations of HF stem cells (HFSCs) that reside within the bulge. In contrast, the SG is found to be in a constant state of renewal and is replenished by distinct pools of SCs or progenitors located in the HF or base of the SG. SGs play a central role in the function of our skin, yet very little is known regarding the mechanisms of its regulation. Furthermore, SGs can give rise to various pathologies including acne, and SG carcinomas, which can metastasize and result in high mortality. Thus, there is an immediate need for generating an ex vivo model for specifically studying and manipulating the SG.

An important population that has been reported to govern SG homeostasis is defined by the expression of B-lymphocyte-induced nuclear maturation protein 1 (Blimp1). These Blimp1⁺ cells reside at the base of the SG and function as a sebocyte progenitor population (Donati, G. & Watt, F. M. Stem cell heterogeneity and plasticity in epithelia. Cell stem cell 16, 465-476 (2015).). Alternatively, it has recently been suggested that Blimp1 functions in terminally differentiated sebocytes as well as in granular IFE cells. An ex vivo model for the SG however, still has yet to be generated.

### SUMMARY OF THE INVENTION

The present invention provides sebaceous gland-like organoids comprising Blimp1 positive cells are provided. Methods of producing the organoids are also provided, as are methods for testing a skin drug with the organoid.

According to a first aspect, there is provided a sebaceous gland (SG)-like organoid, comprising:
a. an outer layer of cells, wherein the outer layer comprises a proliferating cell, a stem cell and a B-lymphocyte-induced nuclear maturation protein 1 (Blimp1) expressing cell; and
b. an inner core of non-proliferating cells wherein the inner core comprises a Blimp1 expressing cell.

According to another aspect, there is provided a method of producing an SG-like organoid, the method comprising culturing a Blimp1 expressing skin stem cell in a 3D growth matrix for a time sufficient for the production of the SG-like organoid.

According to some embodiments, the outer layer cells express Keratin 5 (K-5). According to some embodiments, the outer layer cells express K-5 at a higher level than the inner core cells. According to some embodiments, the outer layer comprises cells expressing keratin 15 (K-15).

According to some embodiments, the organoid is derived from a Blimp1 expressing stem cell. According to some embodiments, the organoid is devoid of immortalized cells, genetically modified cells or both.

According to some embodiments, the outer layer further comprises a stem cell. According to some embodiments, the stem cell expresses Blimp1. In some embodiments, every stem cell of the outer layer expresses Blimp 1.

According to some embodiments, the outer layer comprises cells expressing c-Myc. According to some embodiments, the c-Myc regulates organoid size.

According to some embodiments, the outer layer comprises between 20% and 60% proliferating cells. According to some embodiments, at least one outer layer proliferating cell does not express Blimp 1. According to some embodiments, the outer layer cells migrate to the inner cell core. According to some embodiments, a portion of the outer layer cells divide asymmetrically into an outer layer cell and an inner core cell. According to some embodiments, the organoid is characterized by outer layer cells that migrate to the inner cell core. According to some embodiments, the organoid is characterized by a portion of the outer layer cells that divide asymmetrically into an outer layer cell and an inner core cell

According to some embodiments, the inner core cells are larger than the outer layer cells. According to some embodiments, the inner core comprises sebocyte-like cells. According to some embodiments, the inner core comprises lipid-producing cells. According to some embodiments, the sebocyte-like cells and the lipid-producing cells stain positive with Oil red O (ORO) dye. According to some embodiments, the sebocyte-like cells and the lipid-producing cells comprise a lipid composition similar to in vivo sebocytes.

According to some embodiments, the inner core is devoid of proliferating cells. According to some embodiments, the inner core Blimp1 expressing cells are terminally differentiated.

According to some embodiments, the sebocyte-like cells and the lipid-producing cells express at least one sebocyte marker. According to some embodiments, the sebocyte marker is selected from the group consisting of androgen receptor (AR), fatty acid synthase (FASN), peroxisome proliferator-activated receptor beta (PPAR-β) and peroxisome proliferator-activated receptor gamma (PPAR-γ).

According to some embodiments, the organoid is ex-vivo. According to some embodiments, the organoid is in a Matrigel.

According to some embodiments, the SG-like organoid is an SG-like organoid of the invention.

According to some embodiments, the Blimp1 expressing skin stem cell is a primary epidermal keratinocyte. According to some embodiments, the epidermal keratinocyte expresses integrin alpha 6 (α6). According to some embodiments, the epidermal keratinocyte does not express lymphocyte antigen 6 complex (SCA-1). According to some embodiments, the method further comprises preculturing the primary epidermal keratinocyte stem cell on a feeder layer before the culturing in a 3D growth matrix.

According to some embodiments, the method further comprises providing Blimp1 expressing mitotic skin cells. According to some embodiments, the providing comprises FACS sorting of skin cells.

According to some embodiments, only Blimp1 expressing skin cells are cultured in the 3D growth matrix.

According to some embodiments, the 3D growth matrix is Matrigel.

According to some embodiments, the culturing comprises addition of media, and wherein the media is hair follicle stem cell media. According to some embodiments, the culturing is in media comprising 45-55 mM Ca2+. According to some embodiments, the culturing is in media comprising at least one growth factor selected from epidermal growth factor (EGF), fibroblast growth factor (FGF), Noggin and R-spondin. According to some embodiments, the media comprises EGF, FGF, Noggin and R-spondin.

According to some embodiments, the method of the invention further comprises preculturing a Blimp1 expressing mitotic skin cell on feeder cells for at least 8 passages and transferring at least one Blimp1 expressing mitotic skin cell to a 3D growth matrix. According to some embodiments, the feeder cells are J2 feeder cells.

According to some embodiments, the culturing is in the presence of an agent that inhibits cell-cell interaction-dependent anoikis. According to some embodiments, the culturing is in media comprising a ROCK inhibitor. According to some embodiments, the ROCK inhibitor is Y27632.

According to some embodiments, the method is performed ex vivo.

According to some embodiments, the time sufficient for the production of the SG-like organoid is at least 8 days.

According to some embodiments, the method further comprises coculturing a Thy1.2 expressing stem cell in the 3D growth matrix with the Blimp1 expressing skin cell. According to some embodiments, the Thy1.2 expressing stem cell is an interfollicular epidermis (IFE) stem cell. According to some embodiments, the Thy1.2 expressing stem cell is a keratinocyte

According to another aspect, there is provided a method of ex vivo testing a skin drug, the method comprising contacting a SG-like organoid of the invention with the skin drug. According to some embodiments, the skin drug is for treating acne.

According to some embodiments, the method further comprises contacting the SG-like organoid with at least one of dihydrotestosterone androgen (DHT), PPAR-γ BRL-49653 activator (BRL) and linoleic acid (LIN) before contacting with the skin drug. According to some embodiments, the contacting is with DHT, BRL and LIN.

According to another aspect, there is provided a method of treating acne in a subject in need thereof, the method comprising decreasing c-Myc expression in a SG of the subject, thereby treating acne in the subject.

According to another aspect, there is provided use of a pharmaceutical composition comprising a c-Myc inhibitor for treating acne in a subject in need thereof.

According to some embodiments, the decreasing comprises contacting the SG with a c-Myc inhibitor. According to some embodiments, the decreasing is in the inner lipid-producing cells of the SG.

According to some embodiments, the c-Myc inhibitor is 10058-F4.

According to some embodiments, the pharmaceutical composition is configured for topical administration.

According to some embodiments, the treating comprises at least one of decreasing the size of the SG; decreasing lipid production by the SG; and decreasing transcription of PPAR-γ, Cyclin D or both.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-P: Blimp1−YFP⁺ cells generate a sebaceous gland-resembling structure in 3D culture. (1A-B) Micrograph of dorsal skin wholemount extracted from P56 (telogenic)-old B6. Cg-Tg(Prdm1−EYFP) 1Mnz/J (denoted Blimp1−YFP) mouse stained for Oil Red O (ORO). (1A) Blimp1−YFP⁺ signal (designated with yellow arrows) can be seen at the base of the sebaceous gland, (1B) in mature sebocytes (designated by pink arrow), and differentiated keratinocytes in the interfollicular epidermis. Scale bars: 20 μm. (1C) Dot plots of fluorescent-activated cell sorting (FACS) analysis employing Blimp1−YFP reporter mice demonstrating the α6⁺; Sca1⁻; Blimp1−YFP⁺ population [n=5 pooled mice]. (1D-E) Micrographs of Image stream analysis showing brightfield (BF) images of Blimp1−YFP⁺ cells, indicating that these cells display three distinct morphologies: 1) small cells with high circularity, 2) large cells resembling sebocytes and 3) differentiated keratinocytes. Scale bars: 10 μm. (1F) Micrograph of 2D culture of α6⁺; Sca1⁻; Blimp1−EYFP⁺ cells at passage 10. (1G) A schematic diagram representing the process of Blimp1−YFP⁺-derived organoid generation. (1H) Time course of a single α6⁺; Sca1⁻; Blimp1−EYFP⁺ cell maintained in 3D culture for a period of twelve days. Scale bars: 20 μm. (1I) A micrograph of a single-cell suspension of α6⁺; Sca1⁻; Blimp1−EYFP⁺ cells, supplemented with growth factors, which forms a large number of organoids after a period of one week. Scale bars: 100 μm. (1J) A micrograph of 3D culture of α6+; Sca1⁻; Blimp1−EYFP⁺-derived organoids grown in Matrigel without supplemented growth factors. (1K) A bar chart of organoid forming efficiency of cultured α6⁺; Sca1⁻; Blimp1−EYFP⁺ cells, grown in the presence or absence of growth factors (GFs). (1L) 3D reconstructed confocal image of an organoid after 10 days in culture stained for actin. First panel shows an external view, revealing a layer of compact cells (Scale bars: 50 μm), while the other panels show cross-sections demonstrates large cells of varying sizes. (1M-N) 3D culture of (1M) α6⁺; CD34⁺ and (1N) α6⁺; ScaI⁺ cells in Matrigel. (1O) Micrographs of a 3D culture of isolated α6⁺; ScaI⁻; Blimp1−EYFP⁺ cells directly seeded in Matrigel, supplemented with GFs and ROCK inhibitor Y-27632 after 7 and 14 days. (1P) Bar chart of organoid forming efficiency from isolated α6⁺; ScaI⁻; Blimp1−EYFP⁺ cells directly seeded in Matrigel, supplemented with GFs and grown in the presence or absence of ROCK inhibitor Y-27632. ***p<0.001.

FIGS. 2A-O: Expression patterns of sebaceous gland markers in sebaceous glands and organoids. (2A) A schematic view of the sebaceous gland structure. (2B) Tail skin wholemount extracted from P56 (telogenic)-old wild type (WT) mouse stained for K15 and Ki67. (2C) A micrograph of confocal analysis of Blimp1−YFP⁺ generated organoids cultured for 10 days, stained for Ki67. (2D) A bar graph showing the percentages of Ki67+ cells in SGs of P56 (telogenic)-old wild type (WT) mice and Blimp1−YFP⁺-derived organoids cultured for 10 days. Data are represented as mean±SEM (n=60). NS means no significance. (2E) A micrograph of confocal analysis of Blimp1−YFP⁺ generated organoids cultured for 10 days, stained for MCM2. (2F) A bar graph showing the percentages of MCM2⁺ cells in SGs of P56 (telogenic)-old wild type (WT) mice and Blimp1−YFP⁺-derived organoids cultured for 10 days. (2G-H) Micrographs of confocal analysis of (2G) Blimp1−YFP⁺ generated organoids cultured for 10 days, and (2H) dorsal skin (left panels) and tail skin (right panels) whole mounts extracted from P56 (telogenic)-old wild type (WT) mouse stained for K5 and K15. Scale bars: 20 μm. (2I) Micrographs of confocal analysis of Blimp1−YFP⁺ generated organoids cultured for 10 days, showing Blimp1−YFP⁺ cells in the proliferating outer layer and in the inner compartment as shown in organoid cross sections. Scale bars: 50 μm. (2J-K) Fluorescent micrographs of BrdU pulse-chase analysis for (2J) 24 hours, (2K) 48 hours and 72 hours showing migration of BrdU-labeled cells from the outer layer to the inner non-proliferating mass. (2L-M) Fluorescent micrographs of BrdU pulse-chase analysis in 7 day old Blimp1−YFP⁺ cell-derived organoids, chased over (2L) 24 hours, (2M) 48 hours and 72 hours. (2N-O) Micrographs of live imaging of movement kinetics, utilizing light sheet microscopy, examining day 7 organoids derived from Blimp1−YFP-H2B-GFP (nuclear labeled) over a (2N) 24 hour and (2O) 7-day period. (2N) Two tracked cells and their progeny (blue and red dots, respectively) over time are marked. (2O) Analysis of cell movement was performed and each nucleus was marked as a spheroid. Spatial movement was tracked and represented as a vector. Scale bars: 10 μm (2N, 2O), 20 μm (2B, 2C, 2E, 2G, 2M), 50 μm (2J, 2L).

FIGS. 3A-F: Lipidomics analysis of Blimp1−YFP⁺ cell-derived organoids. (3A) A micrograph of Blimp1−YFP⁺-derived sebaceous gland organoids lipid stained with oil red O (ORO). Scale bars: 50 (3B) Base peak chromatogram of Sebaceous gland (SG) sample. Lipids specifically abundant in the three SG-like samples: 1) extracted SGs (SG), 2) SG organoids (SG Org) and 3) Blimp1−YFP⁺ (Blimp1) cells grown in 2D culture, are denoted with red. Several most abundant lipids are denoted with blue. (3C) Representative RT/mz map for triacylglycerides (TGs) peak positions across all superimposed LC/MS samples. Each dot represents the center of a peak group for an identified TG compound. Arrows join peaks separated by mass difference of C₂H₄ fragment (5 ppm mass precision) sharing the same number of double bonds. (3D) Heat map of normalized intensities of lipid compounds identified in the samples. Eighteen columns are arranged by unsupervised hierarchical clustering, while rows are composed according to k-means clustering outcome (three clusters: SG/SG-Org/Blimp (cluster I; red), epidermis/SG/SG-Org (cluster II; green) and epidermis/HEK293T cells (HEK)/brain (cluster III; blue)). For each sample type, three biological replicates were analyzed. PCA loadings plot showing two major directions identified among samples accounting for more than half of the total variance. The main effect is associated with SG features (SG-like), where loadings are close between SG, SG Org, and Blimp1 samples. The second strongest effect is epidermis-like (Epi-like) profile, where loadings are close between epidermis and SG. (3E) PCA loadings plot shows three major directions identified among samples accounting for 75% of total variance. The main effect is associated with SG features, where loadings are close between SG, SG Org, and Blimp1 samples. The second strongest effect is epidermis-like profile, where loadings are close between epidermis and SG features. (3F) Density plots of PCA scores showing separation of total lipidome into two major groups of compounds: SG-specific and the rest of the lipids, as highlighted by color. Density of scores shows non-continuous distribution supporting distinct lipidome profiles for the denoted groups of samples.

FIGS. 4A-O: c-Myc regulates proliferation and differentiation in the sebaceous gland organoid. (4A) Micrographs of dorsal skin whole mount extracted from P56 (telogenic)-old wild type (WT) mouse stained for Ki67 and cMyc. (4B-C) Micrographs of confocal analysis of Blimp1−YFP⁺ cell-derived organoids cultured for 10 days, stained for (4B) Ki67 and cMyc and (4C) MCM2 and cMyc, indicates proliferating cells only in the outer layer of the SG and SG organoids. Scale bars: 20 μm (4A-C). (4D-N) Micrographs and bar charts of Blimp1−YFP⁺-derived sebaceous gland organoids cultured for six days and treated for four days with cMyc inhibitor 10058-F4, or DMSO as control. (4D) Brightfield micrographs showing treated organoids display decreased organoid size compared to control organoids. (4E) Micrographs of confocal analysis of treated and control organoids stained for actin. (4F) Bar chart of organoid size in treated organoids compared to control organoids. Data is represented as the mean±SEM relative to control organoids (n=150 organoids). (4G) Bar chart of cell size of treated and control organoids (n=80), measuring cell diameter of inner cells and cells at the outer layer. (4H) Micrographs of cross sections of control and treated organoids stained for actin. (4I) Bar chart of cell number in the outer layer and inner mass of control and treated organoids. Data is represented as the mean±SEM (n=60). (4J-K) Micrographs of confocal analysis of treated and control organoids stained with (4J) K5 and Ki67 and (4K) MCM2, showing dramatic decrease in proliferating cells upon cMyc inhibition. (4L) Bar charts of the average percentage of proliferating cells, as marked by Ki67 and MCM2, per control or treated organoid. Data is represented as the mean±SEM (n=60). (4M-N) Micrographs of brightfield cross sections of control and treated organoids stained for Oil Red O (ORO), showing diminished differentiation upon cMyc inhibition. (4O) Bar charts of quantitative RT-PCR analysis of treated organoids. mRNA levels of known sebocyte differentiation markers including androgen receptor (AR), fatty Acid Synthase (FASN) and peroxisome proliferator-activated receptors beta and gamma (PPAR-β,γ) are significantly decreased upon cMyc inhibition. Changes in cycle threshold values were normalized to RPL0. Data are represented as mean±SEM relative to control organoids (n=3 independent wells). Scale bars: 50 μm (4E, 4G, 4H, 4J, 4K, 4M), 200 μm (4D). *p<0.05, **p<0.005, ***p<0.001.

FIGS. 5A-V: Thy1.2 marks a distinct population of epidermal cells. (5A) A schematic view of the hair follicle and epidermis structure. (5B) Whole-mount micrograph of a Brainbow 2.1 embryo at E15.5 (5C) Micrograph of confocal Z-stack of a Brainbow 2.1 placode at E15.5. (5D) Micrograph of a section of a E15.5 embryo dorsal skin stained for Thy1.2. (5E) Micrograph of dorsal skin whole-mount extracted from P1-old wild type (WT) mouse stained for Thy1.2 during hair peg development. (5F) Micrograph of dorsal skin whole-mount extracted from P1-old WT mouse stained for Thy1.2 in the interfollicular epidermis (IFE). (5G) Micrograph of dorsal skin whole-mount extracted from P56 (telogen)-old WT mouse stained for Thy1.2 and Ki67 shows homogenous distribution of Thy1.2+ cells in the dorsal skin IFE. (5H) Schematic representation of average distance in micrometers of a cluster of surrounding Thy1.2+ cells (red) to the central Thy1.2+ cell (orange) (n=30). (5I) Micrograph of tail skin whole-mount extracted from P56-old WT mouse stained for Thy1.2 indicates non-homogenous distribution of Thy1.2+ cells in the tail skin IFE. (5J) Micrograph of exposed IFE of tail skin whole-mount extracted from P56-old WT mouse shows Thy1.2+ cells restricted to label-retaining cells (LRC) areas. The dashed line demarcates non-LRC areas. (5K) A schematic view of tail skin epidermis indicating LRC and non-LRC regions relative to hair follicles. (5L) Micrographs showing confocal analysis of dorsal skin whole-mount extracted from P56-old WT mouse stained for Thy1.2 and CD104 showing Thy1.2+ cells at the basal layer. (5M-N) Micrograph of dorsal skin whole-mount extracted from P56-old WT mouse stained for Thy1.2 in the IFE. (5O) Micrograph of dorsal skin whole-mount extracted from P56-old WT mouse stained for Thy1.2+ and Ki67+ cells in the sebaceous gland (SG) base and infundibulum (IFD). (5P) Micrograph of tail skin whole-mount extracted from P56-old WT mouse stained for CD104 and Thy1.2. (5Q) Micrograph of confocal analysis of tail skin whole-mount extracted from P56-old WT mouse, stained for Thy1.2 and CD104, shows Thy1.2+ cells in the basal layer. (5R) Micrograph of dorsal skin whole-mount extracted from P56-old WT mouse stained for Thy1.2 and Ki67. (5S) Pie charts of the percentage of Thy1.2+ cells positive for Ki67 or BrdU in dorsal skin and tail skin IFE (n=800). (5T) Micrograph of dorsal skin whole-mount extracted from P56-old WT mouse stained for Thy1.2 and Ki67. (5U) Micrograph of tail skin whole-mount extracted from P56-old WT mouse stained for Thy1.2 and Ki67 shows minimal co-localization. (5V) Micrograph of dorsal skin whole-mount extracted from P56-old WT mouse after a 24h BrdU chase stained for Thy1.2 and BrdU shows minor overlap. Scale bars: 20 um (5C, 5Q, 5L, 5R), 50 um (5D, 5F, 5K-L, 5T, 5U-V), 100 um (5E, 5G, 5J, 5O-P), 200 um (5I). Denotations: DAPI, 4′,6-diamidino-2-phenylindole; LRC, label retaining cells; IFE, interfollicular epidermis; BrdU, 5-bromo-2-deoxyuridine; YFP, yellow fluorescent protein; GFP, green fluorescent protein

FIG. 6A-S: Characterization of Thy1.2^{high} epidermal cells. (6A) Micrograph of dorsal skin extracted from P56-old WT mouse stained for Thy1.2 and Lrig1 indicating that Thy1.2+ cells are distinct from the Lrig1+ population. (6B-C) Contour plots of FACS employing B6.Cg−Tg(Prdm1−EYFP)1Mnza (Blimp-YFP) and B6.129P2-Lgr6tm2.1(cre/ERT2)Clea (Lgr6-GFP) reporter mice showing no overlap with Thy1.2 high population. (6D) Micrograph of dorsal skin whole-mount extracted from P56-old wilt type (WT) mouse immunostained for Axin2 and Thy 1.2 in the skin IFE. (6E) Micrograph of negative control staining with secondary antibodies Alexa Fluor-488 and Alexa Fluor-546 for Thy1.2 and Axis2, respectively, in the IFE. (6F) A pie graph of the percentage of Axin2+ cells positive for Thy1.2 in dorsal skin IFE (n=400). (6G-H) Micrographs of dorsal skin whole-mount extracted from P56-old WT mice shows Axin2+ cells at the hair follicle bulge. Inset shows the same image without brightfield. (6I) Micrographs of tail skin whole-mount extracted from P56-old WT mouse stained for Axin2. Inset shows negative control staining with secondary antibody only. (6J) Contour plots of FACS analysis of dorsal skin from P56-old WT mice (n=5) using antibodies for integrin α6, integrin (31, Sca-1, CD34 and Thy1.2. α6+(31+Cd34−Scal1−Thy1.2high (pink), α6+(31+Cd34+Scal1−Thy1.2− (blue) and α6+β1+Cd34−Scal1+Thy1.2low (purple). (6K) Image stream analysis showing brightfield images of α6+β1+Cd34+Scal1− and α6+Thy1.2high Sca1− cells, showing that the α6+Thy1.2high Sca1− cells display high circularity. (6L) Micrograph of fluorescent image of α6+Thy1.2high Sca1− indicating that these cells express Thy1.2 through the membrane and lack CD34. (6M) Micrograph of sorted α6+CD34+Scal−Thy1.2−(bulge) and α6+CD34−Sca1−Thy1.2high cells were grown with sustaining J2 feeder cells. Bright field phographs show cells two weeks post-plating. (6N) Bar graphs of the number of colonies (upper) and fold increase (lower) during long term passaging. X-axis shows passage (P) number. Error bars represent SEM. (6O) Micrograph of Oil red O (ORO) staining of α6+CD34−Sca1−Thy1.2high showing that α6+CD34−Sca1−Thy1.2high can produce differentiated sebocytes in vitro. (6P) Bar plot of log 2-fold ratios of FPKM values for a selected number of markers comparing α6+CD34−Sca1−Thy1.2high and α6+CD34+Sca1−Thy1.2− populations. (6Q) Heatmap of absolute gene expression (FPKM values) based on RNA-seq data comparing α6+CD34+Sca1−Thy1.2− (a6+CD34+) and α6+CD34−Sca1−Thy1.2high (α6+Thy1.2high) populations. Genes are clustered according to expression similarity. (6R) Scatterplot of log 2 FPKM expression values of RNA-seq data from α6+CD34+Sca1−Thy1.2− versus α6+CD34−Sca1−Thy1.2high. Log4-fold upregulated genes are marked red; log 4-fold downregulated genes are marked blue. (6S) Ven diagram summarizing the number of genes with less than log 4-fold changes in expression (grey), higher than log 4-fold expression (red) and lower than log 4-fold expression (blue). 20 um (6D-E, 6G-H, 6O), 50 um (6A, 6I), 100 um (6M).

FIGS. 7A-R: Thy1.2 high cells can generate the IFE. (7A) Schematics of the Thy1.2 promoter driving Cre-ERT2 expression (upper), Brainbow 2.1 construct encoding four fluorescent proteins driven by the strong CAGG promoter in the Rosa26 locus (middle) and upon Cre recombination, the Neomycin (Neo) cassette is removed and the multicolor construct recombines randomly to result in four possible outcomes with different fluorescent proteins being expressed in cells and their progeny. Upon Tamoxifen (TMX) treatment Cre-ERT2 fusion protein can translocate into the nucleus. (7B) Micrograph of confocal analysis of representative TMX-Cre-ERT2-targeted clones at day 1. (7C) Micrograph of a cross section of dorsal skin extracted from Thy-Cre; Rosa26-Confetti mice at E15.5 showing expression in the placode. (7D-F) Micrographs of confocal images of tail skin whole-mounts of Thy-Cre; Rosa26-Confetti mice extracted at P1 and P3 indicating that Thy1.2high cells can contribute to the (7D) interfollicular epidermis and (7E-F) hair follicle. (7G-K) Micrographs of confocal images of tail skin whole-mounts indicating that Thy1.2high cells can generate the (7G-H) sebaceous gland, (7I-J) hair follicle, (7J-K) interfollicular epidermis, and (7K) infundibulum. (7L) Bar graph showing that the size distribution of clones comprised of more than one labelled cell at 24 hours PI diminishes according to basal and total cell number (n=57 clones). (7M-N) Micrograph of confocal analysis of representative TMX-Cre-ERT2-targeted clones at (7M) day 7, (7N) day 16, (7O-P) day 30, and (7Q) 1 year post induction (PI). (7R) Micrographs of representative section of multiple optical sections from a Z-stack acquisition of dorsal skin interfollicular epidermis (IFE) at 30 days PI. White lines mark polygonal projection of a cornified layer cell termed a polygonal projection unit (PPU). Scale bars: 10 um (7R), 20 um (7B, 7H), 50 um (7C-D, 7G, 7I, 7K, 7M-O), 100 um (7E-F, 7J, 7P-Q).

FIGS. 8A-J: Lineage tracing in the inducible Thy1.2CreERT2-Confetti mouse. (8A) A bar graph showing events of successful label initiation per area containing 20 hair follicles (HFs) 3 days post Tamoxifen (TMX) administration in the dermal papilla (DP), hair germ (HG), isthmus, hair follicle (HF) bulge, sebaceous gland (SG) base, infundibulum (IFD) and interfollicular epidermis (IFE). (8B-J) Micrographs of confocal analysis of representative CreERT2-targeted clones at (8B) day 1, (8C) day 5, (8D-E) day 16 and (8F-J) day 30 post TMX induction. Scale bars: 20 um (8B, 8E), 50 um (8C-D, 8F), 100 um (8G-I, 8J).

FIGS. 9A-G: Long term lineage tracing indicate that Thy1.2high cells can give rise to all layers of the interfollicular epidermis. (9A-C) Micrographs of Z-stack 3D reconstruction of interfollicular epidermis (IFE) of dorsal skin whol-mount 30 days post Tamoxifen (TMX) administration from the (9A) basal side and (9B) the cornified side. (9D-F) Micrographs of confocal analysis of dorsal skin whole-mount (9D) 60, (9E) 120 days and (9F) 1 year post induction (PI). (9G) A graph of basal footprint per polygonal projection unit (PPU) in the basal layer. Measurements represent number of labelled basal cells in eah PPU of surviving clones as imaged by confocal microscopy on dorsal skin from day 1 to 1 year PI (n=50). Scale bars: 50 um (9A-E), 100 um (9F).

FIGS. 10A-T: Thy1.2+SCs contribute to epidermal wound repair. (10A) Micrographs of dorsal skin whole-mount stained for Thy1.2 showing colocalization of induced Thy1.2high cells 3 days post TMX administration. (10B) Micrographs of Z-stack 3D reconstruction of interfollicular epidermis of dorsal skin 30 days post induction showing co-staining of Thy1.2 and labeled cells at the basal layer. (10C) Side view 3D reconstruction of dorsal skin whole-mount 30 days PI. (10D) Micrograph of exposed interfollicular epidermis of dorsal skin extracted from P56-old WT mouse stained for Thy1.2. (10E) Computerized segmentation and identification of individual Thy1.2 marked cells that appear in 10D. (10F) Voronoi diagram showing partitioning of the basal layer into regions that are closest to each cell identified in 10E. (10G-K) Micrographs showing Thy1.2 progeny migrate to the wound in a clonal fashion and can be detected at (10G) day 5, (10H) day 7, (10I) day 30, and (10J-K) day 60 post wound infliction (PWI). (10L-Q) Micrographs of dorsal skin harvested at (10L) day 7, (10M) day 30, (10N-O) day 60, (10P-Q) and day 120 post wound infliction (PWI) in induced animals showing clonal trails emanating towards the wound site. (10R) Micrograph showing at 120 days PWI clonal units can be detected in the neo-epidermis that deviate from the regular polygonal projection unit structure. Arrows indicate co-localization of immunostaining and induced cells at the basal layer. (10S) Confocal analysis of dorsal skin whole-mount 120 days post induction shows polygonal projection units (PPUs) with structured organization. (10T) Confocal analysis of dorsal skin 120 days post wounding reveals abnormal PPU organization within the neo-epidermis. Dashed line demarcates wound area. Scale bars: 10 um (10A, 10B inset), 20 um (10C), 50 um (10D-F, 10I inset, 10K, 10R-T), 200 um (10G-J).

FIG. 11: Sebaceous gland organoid derived from Thy1.2 coculture. Micrographs of SG organoids derived from culture of just Blimp1⁺ cells (top row) and from coculture of Blimp1⁺ cells and Thy1.2+ stem cells (bottom row).

FIGS. 12A-E: The sebaceous gland organoid as a model system. (12A) Brightfield micrographs of SG organoids after incubation with caspase-3 inhibitors zDEVD-fmk and Ivachtin and with DMSO control. (12B) Bar graph quantifying organoid size reduction after treatment with the caspase-3 inhibitors and the anti-acne drug Tretinoin (Ret-Avit). (12C-D) Fluorescent micrographs of SG organoids treated with zDEVD-fmk and stained for proliferation markers (12C) Ki67 and (12D) MCM2. (12E) A bar graph quantifying the reduction in proliferating cells in the SG organoid after treatment with zDEVD-fmk.

FIGS. 13A-R: Sebaceous gland organoids can be used as a model for acne vulgaris. (13A-I) Blimp1−YFP⁺-derived sebaceous gland organoids cultured for five days and treated for three days with a combination of DHT, BRL, LIN (DBL), or pure ethanol as control, followed by treatments for three days with Retinoic acid (RA), 13-cis-Retinoic acid (13cis RA) or Ret-Avit (Tretinoin). (13A) DBL-treated organoids display increased organoid size in compared to control organoids, which is reduced upon treatments with Retinoic acid derivatives. (13B) Statistical analysis of treated organoids size compared to control organoids. (n=150 organoids). (13C) Bar chart of real time PCR analysis of mRNA levels of androgen receptor (AR), Fatty Acid Synthase (FASN), peroxisome proliferator-activated receptors gamma (PPAR-γ) and beta (PPAR-β) are significantly increased upon combined treatment (DBL). (13D) Organoids stained for Oil Red O (ORO) show increased lipid production upon DBL treatment and Retinoic acid derivatives. (13E) Bar chart of the number of Oil red O (ORO) positive cells in combined DBL treatment compared to control organoids (n=120). (13F) Confocal analysis of treated and control organoids stained with Ki67, showing increased proliferation upon androgen induction and decreased proliferation in retinoic acid derivatives-treated organoids. (13G) Bar chart showing mRNA levels of cMyc and Cyclin-D1 are significantly increased upon combined treatment. (13H) Bar chart showing DBL-treated organoids display increased organoid size in comparison to control organoids, which is reduced upon treatments with retinoic acid derivatives. (13I) Quantitative real time (RT)-PCR analysis of treated organoids showing changes in mRNA levels of cMyc, Cyclin D1, androgen receptor (AR), fatty Acid Synthase (FASN) and peroxisome proliferator-activated receptors beta and gamma (PPAR-β,γ). (13J) Bar charts showing the number of Ki67+ proliferating cells indicates increased proliferation in DBL-treated organoids, which is significantly reduced upon treatments with retinoic acid derivatives (n=100). (13K-R) Blimp1−YFP⁺-derived sebaceous gland organoids cultured for five days and treated for three days with DBL combination as before, following treatments for three days with c-Myc inhibitor 10058-F4. (13K) c-Myc inhibitor-treated organoids display decreased organoid size in compared to DBL-treated organoids. (13L) Bar chart showing the size of treated organoids compared to control organoids. (13M) Organoids stained for ORO, showing decreased lipid production upon c-Myc inhibition. (13N) Bar chart showing the number of ORO+ cells in c-Myc inhibited organoids compared to DBL-treated organoids. (13O) Bar chart showing analysis of treated and control organoids, measuring cell diameter of inner cells. (13P) Quantitative RT-PCR analysis of treated organoids showing changes in mRNA levels of c-Myc, Cyclin D1, androgen receptor (AR), fatty Acid Synthase (FASN) and peroxisome proliferator-activated receptors beta and gamma (PPARβ,γ). (13Q) Bar chart showing the number of Ki67+ proliferating cells in c-Myc inhibitor-treated organoids compared to DBL-treated organoids, showing significant reduction upon treatments with the inhibitor. (13R) Confocal analysis of treated and control organoids stained with Ki67, showing decreased proliferation in c-Myc inhibited organoids. In all RT-PCR experiments, changes in cycle threshold values were normalized to Rplp0. Data are represented as mean relative to control organoids (n=3 independent wells). Error bars show ±s.e.m. Data are represented as mean, error bards show ±s.e.m. Significance was determined using two-tailed unpaired Student's t test where *p<0.05, **p<0.005, ***p<0.001. Scale bars: 50 μm.

### DETAILED DESCRIPTION OF THE INVENTION

The present invention, in some embodiments, provides a sebaceous gland-like organoid comprising Blimp1 positive cells. Methods of producing the organoid are also provided, as are methods for testing a skin drug with the organoid.

The invention is based on the surprising finding that a single Blimp1 positive cell is capable of generating a highly organized sebaceous gland (SG)-like organoid. A single stem cell is capable of generating a highly complex organ with an outer proliferating layer of cells, that are capable of asymmetric division into a new stem cell and a lipid-producing cell which migrates into the non-proliferating center of the organoid. The lipid-producing cells are highly sebocyte-like and secrete a lipid composition similar to that of endogenous sebocytes. Interestingly, the SG-organoid can be improved by culturing the Blimp1+ cell with Thy1.2+ interfollicular epidermis (IFE) stem cells.

By a first aspect, there is provided an organoid, comprising
a. an outer layer of cells, wherein the outer layer comprises a proliferating cell and a B-lymphocyte-induced nuclear maturation protein 1 (Blimp1) expressing cell; and
b. an inner core of non-proliferating cells wherein the inner core comprises a Blimp1 expressing cell.

By another aspect, there is provided a method of producing an SG-like organoid, the method comprising culturing a Blimp1 expressing cell in a growth matrix for a time sufficient for the production of the organoid.

As used herein, an “organoid” refers to a simplified version of an organ produced in vitro. In some embodiments, the organoid is smaller than the in vivo organ. In some embodiments, the SG-like organoid is an SG-like organoid of the invention. In some embodiments, the organoid has a microanatomy or cellular organization similar to the organ. In some embodiments, the cellular organization is 2D organization. In some embodiments, the cellular organization is 3D cellular organization. In some embodiments, the organoid functions similarly to the organ. In some embodiments, the organoid secretes similar to the organ. In some embodiments, the organoid signals similar to the organ. In some embodiments, the organoid responds to a drug or compound similar to the organ. In some embodiments, the organoid is useful for testing a drug to be used on the organ. In some embodiments, the organoid is useful for testing a side effect of a drug or compound of the organ. In some embodiments, the organoid is useful for modeling a disease of the organ. In some embodiments, the organoid self-renews. In some embodiments, the organoid is not immortalized. In some embodiments, the organoid is derived from a stem cell. In some embodiments, the organoid is derived from a single stem cell. In some embodiments, the stem cell is a Blimp1 expressing stem cell. In some embodiments, the stem cell is an epithelial stem cell. In some embodiments, the stem cell is a keratinocyte stem cell.

As used herein, the term “stem cell” refers to a non-differentiated cell which without exogenous manipulation is capable of replicating indefinitely and giving rise to indefinitely more non-differentiated and differentiated cells. In some embodiments, the stem cell is an adult stem cell. In some embodiments, the stem cell is not an embryonic stem cell. Adult stem cells are able to produce differentiated cells of a particular lineage, i.e. epithelial stem cells produce cells differentiated into the epithelial lineage. Generally, stem cells also have one or more of the following properties: an ability to undergo asynchronous, or symmetric replication that is where the two daughter cells after division can have different phenotypes; extensive self-renewal capacity; and clonal regeneration of the tissue in which they exist. “Progenitor cells” differ from stem cells in that they typically do not have the extensive self-renewal capacity. Stem cells are not cells that have been immortalize, but rather cells that naturally can divide indefinitely. Methods of identifying stem cells are well known in the art and include, but are not limited to, identification of cells that express a stem cell surface marker, identification of cells that express a stem cell transcriptional marker and repeated growth in culture to remove differentiated cells. Examples of skin stem cell surface markers include, but are not limited to CD34, CD200, and Integrin alpha 6 (a6). Transcriptional markers of stem cells include, but are not limited to SOX-9, SOX-2, GATA-6, c-myc and MYB.

In some embodiments, the organoid is not genetically manipulated. In some embodiments, the organoid comprises stem cells. In some embodiments, the organoid comprises diploid cells. In some embodiments, the organoid consists of diploid cells. In some embodiments, the organoid is devoid of aneuploid cells. In some embodiments, the organoid is devoid of hyperploid cells. In some embodiments, the organoid is devoid of SZ95 cells. In some embodiments, the organoid is devoid of viral proteins. In some embodiments, the organoid is devoid of Simian virus 40-T antigen.

In some embodiments, the organoid is ex vivo. In some embodiments, the organoid is in vivo. In some embodiments, the method is performed ex vivo. In some embodiments, the method is performed in vivo. In some embodiments, the organoid is in a Matrigel. In some embodiments, the organoid is in a 3D scaffold. In some embodiments, the organoid is in a 3D growth matrix. In some embodiments, the organoid is a sebaceous gland (SG) organoid. In some embodiments, the organoid is a SG-like organoid.

In some embodiments, the organoid is highly organized. In some embodiments, the organoid comprises and outer layer of cells and an inner core of cells. In some embodiments, both the outer layer and core comprise Blimp1 expressing cells. In some embodiments, the Blimp1 expressing cells are Blimp1 highly expressing cells. In some embodiments, the cells are stem cells. In some embodiments, the cells are derived from stem cells. In some embodiments, the Blimp1 expressing cells are stem cells. In some embodiments, the Blimp1 expressing cells of the outer layer are stem cells. In some embodiments, the Blimp1 expressing cells of the outer layer are proliferating cells. In some embodiments, the cells are not genetically manipulated. In some embodiments, the cells are devoid of viral proteins. In some embodiments, the cells do not express Simian virus 40-T antigen. In some embodiments, the cells are diploid cells. In some embodiments, the cells are not aneuploid cells. In some embodiments, the cells are not hyperploid cells. In some embodiments, the cells are not SZ95 cells. In some embodiments, the cells of the organoid are eukaryotic cells. In some embodiments, the cells of the organoid are mammalian cells. In some embodiments, the cells of the organoid are murine cells. In some embodiments, the cells of the organoid are human cells.

In some embodiments, the organoid comprises at least 10, 15, 20, 25, 30, 35, 40, 45, 50, 55, 60, 65, 70, 75, 80, 85, 90, 95 or 100 cells. Each possibility represents a separate embodiment of the invention. In some embodiments, the organoid comprises at least 50 cells. In some embodiments, the organoid comprises between 10-500, 10-450, 10-400, 10-350, 10-300, 10-250, 10-200, 10-150, 20-500, 20-450, 20-400, 20-350, 20-300, 20-250, 20-200, 20-150, 30-500, 30-450, 30-400, 30-350, 30-300, 30-250, 30-200, 30-150, 40-500, 40-450, 40-400, 40-350, 40-300, 40-250, 40-200, 40-150, 50-500, 50-450, 50-400, 50-350, 50-300, 50-250, 50-200, or 50-150 cells. Each possibility represents a separate embodiment of the invention. In some embodiments, the organoid comprises between 50-350 cells. A skilled artisan will appreciate that as the organoid ages it will increase in size, diameter and cell number. In some embodiments, an organoid grown for between 10-14 days comprises between 50-350 cells. In some embodiments, an organoid grown for 7-9 days comprises between 20-250 cells.

In some embodiments, the organoid comprises a diameter of at least 5, 10, 15, 20, 25, 30, 35, 40, 45, 50, 55, 60, 65, 70, 75, 80, 85, 90, 95 or 100 μm. Each possibility represents a separate embodiment of the invention. In some embodiments, the organoid comprises a diameter of between 5-500, 5-450, 5-400, 5-350, 5-300, 5-250, 5-200, 5-150, 10-500, 10-450, 10-400, 10-350, 10-300, 10-250, 10-200, 10-150, 20-500, 20-450, 20-400, 20-350, 20-300, 20-250, 20-200, 20-150, 30-500, 30-450, 30-400, 30-350, 30-300, 30-250, 30-200, 30-150, 40-500, 40-450, 40-400, 40-350, 40-300, 40-250, 40-200, 40-150, 50-500, 50-450, 50-400, 50-350, 50-300, 50-250, 50-200, or 50-150 μm. Each possibility represents a separate embodiment of the invention. In some embodiments, the organoid comprises a diameter of between 40-200 um. In some embodiments, the organoid comprises a diameter of about 100 um. In some embodiments, the organoid comprises an average diameter of 100 um. A skilled artisan will appreciate than when generating organoids, the exact size and diameter will vary from organoid to organoid. Thus, in producing many organoids the average size can be calculated. In some embodiments, an organoid grown for between 10-14 days comprises a diameter of between 40-200 um. In some embodiments, an organoid grown for 7-9 days comprises a diameter of between 10-100 um.

In some embodiments, the organoid comprises a diameter of at least 3, 4, 5, 7, 10, 15, 20, 25, 30, 35, 40, 45, or 50 cells. Each possibility represents a separate embodiment of the invention. In some embodiments, the organoid comprises a diameter of between 3-50, 3-40, 3-30, 3-25, 3-20, 3-15, 3-10, 4-50, 4-40, 4-30, 4-25, 4-20, 4-15, 4-10, 5-50, 5-40, 5-30, 5-25, 5-20, 5-15, 5-10, 7-50, 7-40, 7-30, 7-25, 7-20, 7-15, or 7-10 cells. Each possibility represents a separate embodiment of the invention. In some embodiments, the organoid comprises a diameter of between 5-15 cells. In some embodiments, an organoid grown for between 10-14 days comprises a diameter of between 5-15 cells. In some embodiments, an organoid grown for 7-9 days comprises a diameter of between 3-10 cells.

In some embodiments, the outer layer is 1 cell thick. In some embodiments, the outer layer is not more than 1, 2, 3, 4, or 5 cells thick. Each possibility represents a separate embodiment of the invention. In some embodiments, the outer layer has a uniform thickness. In some embodiments, the outer layer has a variable thickness. In some embodiments, the cells of the outer layer are small and compact. In some embodiments, the cells of the outer layer are smaller than the cells of the inner core. In some embodiments, the cells of the outer layer comprise a diameter of at most 1, 2, 3, 4, 5, 10, 15, 20, 25, 30, 35, 40, 45 or 50 um. Each possibility represents a separate embodiment of the invention. In some embodiments, the cells of the outer layer comprise a diameter of about 17 um. In some embodiments, the cells of the outer layer comprise an average diameter of 17 um. In some embodiments, the cells of the outer layer comprise a diameter of between 10-25, 10-13, 10-21, 10-19, 10-17, 13-25, 13-23, 13-21, 13-19, 13-17, 15-25, 15-23, 15-21, 15-19, 15-17, 17-25, 17-23, 17-21, or 17-19 um. Each possibility represents a separate embodiment of the invention. In some embodiments, the cells of the outer layer comprise a diameter of between 13-21 um. In some embodiments, the cells of the outer layer comprise an average diameter of between 13-21 um. In some embodiments, the cells of the outer layer comprise a volume of at most 30, 50, 70, 100, 130, 150, 170, 200, 250, 300, 350, 400, 450, 500, 600, 700, 800, 900, 1000, 1200, 1400, 1600, 1800, 2000, 2500, 3000, 3500, 4000, 4500, 5000 μm³. Each possibility represents a separate embodiment of the invention.

In some embodiments, the outer layer comprises proliferating cells. In some embodiments, the outer layer comprises Blimp1+ proliferating cells. In some embodiments, the outer layer comprises Blimp1− proliferating cells. In some embodiments, the outer layer comprises Blimp1⁺ and Blimp1− proliferating cells. In some embodiments, the outer layer comprises a stem cell. In some embodiments, the outer layer comprises stem cells. In some embodiments, the outer layer comprises cells derived from stem cells. In some embodiments, the outer layer comprises a Blimp1+ stem cell. In some embodiments, the Blimp1⁺ cells are stem cells. In some embodiments, the Blimp1+ proliferating cells are stem cells. In some embodiments, the outer layer is devoid of Blimp1+ non-proliferative cells.

In some embodiments, the outer layer comprises at least 10, 15, 20, 25, 30, 35, 40, 45, 50, 55, 60, 65, 70, 75, 80, 85, 90, 95, 97, 99 or 100% proliferating cells. Each possibility represents a separate embodiment of the invention. In some embodiments, the organoid comprises at least 10, 15, 20, 25, 30, 35, 40, 45, 50, 55, 60, 65, 70, 75, 80, 85, 90, 95, 97, 99 or 100% proliferating cells. Each possibility represents a separate embodiment of the invention. In some embodiments, the outer layer comprises between 10-60, 10-55, 10-50, 10-45, 10-40, 10-35, 15-60, 15-65, 15-50, 15-45, 15-40, 15-35, 20-60, 20-55, 20-50, 20-45, 20-40, 20-35, 25-60, 25-55, 25-50, 25-45, 25-40, 25-35, 30-60, 30-55, 30-50, 30-45, 30-40 or 30-35% proliferating cells. Each possibility represents a separate embodiment of the invention. In some embodiments, the organoid comprises between 10-60, 10-55, 10-50, 10-45, 10-40, 10-35, 15-60, 15-65, 15-50, 15-45, 15-40, 15-35, 20-60, 20-55, 20-50, 20-45, 20-40, 20-35, 25-60, 25-55, 25-50, 25-45, 25-40, 25-35, 30-60, 30-55, 30-50, 30-45, 30-40 or 30-35% proliferating cells. Each possibility represents a separate embodiment of the invention. In some embodiments, the outer layer comprises at least 20% proliferating cells. In some embodiments, the outer layer comprises at least 25% proliferating cells. In some embodiments, the outer layer comprises at least 30% proliferating cells. In some embodiments, the outer layer comprises between 20-60% proliferating cells. In some embodiments, the outer layer comprises between 20-40% proliferating cells. In some embodiments, the outer layer comprises between 25-55% proliferating cells. In some embodiments, the organoid comprises a similar number (+/−10%) of proliferating cells as compared to the in vivo organ. In some embodiments, the outer layer of the organoid comprises a similar number of proliferating cells as compared to the outer layer of the in vivo organ. In some embodiments, similar or comparable is within +/−10% of the compared to or similar value. Identifying proliferating cells is well known in the art. Methods such as, Ki67 staining, BrdU staining, and MCM2 staining as well as flow cytometry may be used to determine the percentage of proliferating cells.

In some embodiments, the outer layer comprises at least 1, 3, 5, 7, 10, 15, 20, 25, 30, 35, 40, 45, 50, 55, 60, 65, 70, 75, 80, 85, 90, 95, 97, 99 or 100% stem cells. Each possibility represents a separate embodiment of the invention. In some embodiments, the organoid comprises at least 1, 3, 5, 7, 10, 15, 20, 25, 30, 35, 40, 45, 50, 55, 60, 65, 70, 75, 80, 85, 90, 95, 97, 99 or 100% stem cells. Each possibility represents a separate embodiment of the invention. In some embodiments, the outer layer comprises between 1-60, 1-55, 1-50, 1-45, 1-40, 1-35, 1-30, 1-25, 1-20, 1-15, 1-10, 1-7, 1-5, 3-60, 3-55, 3-50, 3-45, 3-40, 3-35, 3-30, 3-25, 3-20, 3-15, 3-10, 3-7, 3-5, 5-60, 5-55, 5-50, 5-45, 5-40, 5-35, 5-30, 5-25, 5-20, 5-15, 5-10, 5-7, 7-60, 1-55, 7-50, 7-45, 7-40, 7-35, 7-30, 7-25, 7-20, 7-15, 7-10, 10-60, 10-55, 10-50, 10-45, 10-40, 10-35, 10-30, 10-25, 10-20, 10-15, 15-60, 15-65, 15-50, 15-45, 15-40, 15-35, 15-30, 15-25, 15-20, 20-60, 20-55, 20-50, 20-45, 20-40, 20-35, 20-30, 20-25, 25-60, 25-55, 25-50, 25-45, 25-40, 25-35, 25-30, 30-60, 30-55, 30-50, 30-45, 30-40 or 30-35% stem cells. Each possibility represents a separate embodiment of the invention. In some embodiments, the organoid comprises between 1-60, 1-55, 1-50, 1-45, 1-40, 1-35, 1-30, 1-25, 1-20, 1-15, 1-10, 1-7, 1-5, 3-60, 3-55, 3-50, 3-45, 3-40, 3-35, 3-30, 3-25, 3-20, 3-15, 3-10, 3-7, 3-5, 5-60, 5-55, 5-50, 5-45, 5-40, 5-35, 5-30, 5-25, 5-20, 5-15, 5-10, 5-7, 7-60, 1-55, 7-50, 7-45, 7-40, 7-35, 7-30, 7-25, 7-20, 7-15, 7-10, 10-60, 10-55, 10-50, 10-45, 10-40, 10-35, 10-30, 10-25, 10-20, 10-15, 15-60, 15-65, 15-50, 15-45, 15-40, 15-35, 15-30, 15-25, 15-20, 20-60, 20-55, 20-50, 20-45, 20-40, 20-35, 20-30, 20-25, 25-60, 25-55, 25-50, 25-45, 25-40, 25-35, 25-30, 30-60, 30-55, 30-50, 30-45, 30-40 or 30-35% stem cells. Each possibility represents a separate embodiment of the invention. In some embodiments, the outer layer comprises at least 1% stem cells. In some embodiments, the outer layer comprises at least 3% stem cells. In some embodiments, the outer layer comprises at least 5% stem cells. In some embodiments, the outer layer comprises at least 7% stem cells. In some embodiments, the outer layer comprises at least 10% stem cells. In some embodiments, the outer layer comprises at least 15% stem cells. In some embodiments, the outer layer comprises at least 20% stem cells. In some embodiments, the outer layer comprises at least 25% stem cells. In some embodiments, the outer layer comprises at least 30% stem cells. In some embodiments, the outer layer comprises between 1-40% stem cells. In some embodiments, the outer layer comprises between 1-20% stem cells. In some embodiments, the outer layer comprises between 3-25% stem cells. In some embodiments, the organoid comprises a similar number (+/−10%) of stem cells as compared to the in vivo organ. In some embodiments, the outer layer of the organoid comprises a similar number of stem cells as compared to the outer layer of the in vivo organ. In some embodiments, similar or comparable is within +/−10% of the compared to or similar value.

In some embodiments, the outer layer comprises Blimp1⁺ cells. In some embodiments, the outer layer comprises a Blimp1+ cell. In some embodiments, the outer layer comprises Blimp1− cells. In some embodiments, the outer layer comprises a Blimp1− cell. In some embodiments, the outer layer comprises Blimp1+ stem cells. In some embodiments, the outer layer comprises stem cells. In some embodiments, Blimp1⁺ cells in the outer layer are stem cells. In some embodiments, the outer layer comprises cells derived from a stem cell. In some embodiments, the Blimp1⁺ cells are derived from a stem cell. In some embodiments, the proliferating cells are derived from a stem cell. In some embodiments, the outer layer comprises cells derived from a primary cell. In some embodiments, the Blimp1⁺ cells are derived from a primary cell. In some embodiments, the proliferating cells are derived from a primary cell. In some embodiments, the outer layer comprises at least 1, 2, 3, 5, 7, 10, 15, 20, 25, 30, 35, 40, 45, or 50% Blimp1⁺ cells. In some embodiments, the outer layer comprises at least 1% Blimp1⁺ cells. Each possibility represents a separate embodiment of the invention. In some embodiments, the organoid comprises at least 1, 2, 3, 5, 7, 10, 15, 20, 25, 30, 35, 40, 45, or 50% Blimp1⁺ cells. Each possibility represents a separate embodiment of the invention. In some embodiments, the organoid comprises at least 1% Blimp1⁺ cells. In some embodiments, the outer layer comprises between 1-30, 1-20, 1-15, 1-10, 1-7, 1-5, 1-3, 2-30, 2-20, 2-15, 2-10, 2-7, 2-5, 2-3, 3-30, 3-20, 3-15, 3-10, 3-7, or 3-5% Blimp1⁺ cells. Each possibility represents a separate embodiment of the invention. In some embodiments, the outer layer comprises between 1-3% Blimp1⁺ cells. In some embodiments, the organoid comprises a similar number (+/−10%) of Blimp1⁺ cells as compared to the in vivo organ. In some embodiments, the outer layer of the organoid comprises a similar number of Blimp1⁺ cells as compared to the outer layer of the in vivo organ.

In some embodiments, the outer layer cells express at least one cytoskeletal protein. In some embodiments, all the outer layer cells express at least one cytoskeletal protein. In some embodiments, a portion of outer layer cells express at least one cytoskeletal protein. In some embodiments, at least 10, 15, 20, 25, 30, 35, 40, 45, 50, 55, 60, 65, 70, 75, 80, 85, 90, 95, 97, 99 or 100% of outer layer cells express at least one cytoskeletal protein. Each possibility represents a separate embodiment of the invention. In some embodiments, the cytoskeletal protein is selected from actin and keratin 5 (K-5). In some embodiments, all the outer layer cells express K-5. In some embodiments, all the outer layer cells express K-5. In some embodiments, the cytoskeletal protein is selected from actin, K-5 and keratin 15 (K-15). In some embodiments, the cytoskeletal protein is actin. In some embodiments, the cytoskeletal protein is K-5. In some embodiments, the cytoskeletal protein is K-15. In some embodiments, the outer layer cells express the cytoskeletal protein at a higher level than do the inner core cells. In some embodiments, the outer layer cells express K-5 at a higher level than the inner core cells. In some embodiments, the outer layer comprises at least one cell expressing K-15. In some embodiments at least 1, 3, 5, 10, 15, 20, 25, 30, 35, 40, 45, or 50% of outer layer cells express K-15. Each possibility represents a separate embodiment of the invention. In some embodiments, between 0.5-10, 0.5-7, 0.5-5, 0.5-3, 0.5-2, 1-10, 1-7, 1-5, 1-3, 1-2, 2-10, 2-7, 2-5, or 2-3% of outer layer cells express K-15. Each possibility represents a separate embodiment of the invention. In some embodiments, between 1-3% of outer layer cells express K-15.

In some embodiments, the outer layer comprises cells expressing c-Myc. In some embodiments, the proliferating cells express c-Myc. In some embodiments, at least 1, 5, 10, 15, 20, 25, 30, 35, 40, 45, or 50% of outer layer cells express c-Myc. Each possibility represents a separate embodiment of the invention. In some embodiments, between 3-50, 3-40, 3-30, 3-25, 3-20, 3-15, 3-10, 5-50, 5-40, 5-30, 5-25, 5-20, 5-15, 5-10, 10-50, 10-40, 10-30, 10-25, 10-20, 10-15, 15-50, 15-40, 15-30, 15-25, or 15-20% of outer layer cells express c-Myc. Each possibility represents a separate embodiment of the invention. In some embodiments, between 10-20% of outer layer cells express c-Myc. In some embodiments, c-Myc regulates organoid size. In some embodiments, c-Myc regulates organoid self-renewal. In some embodiments, c-Myc regulates organoid growth. Monitoring expression of markers is well known in the art. Any assay that employs antibodies specific to c-Myc, actin, K-5 and K-15, such as immunostaining or western blotting for non-limiting example, may be employed. Measuring mRNA of these proteins would also be suitable, and can be performed by RT-PCT, real-time PCR, and the like.

In some embodiments, the outer layer is devoid of lipid producing cells. In some embodiments, the outer layer comprises an epidermal keratinocyte. In some embodiments, the outer layer consists of epidermal keratinocytes. In some embodiments, the outer layer comprises or consists of cells expressing integrin alpha 6 (a6). In some embodiments, the cells expressing α6 are epidermal keratinocytes. In some embodiments, the outer layer is devoid of cells expressing lymphocyte antigen 6 complex (SCA-1). In some embodiments, the epidermal keratinocyte does not express SCA-1.

In some embodiments, at least one cell of the outer layer divides asymmetrically. In some embodiments, a portion of the outer layer cells divide asymmetrically. In some embodiments, the proliferating cells are the portion. In some embodiments, the proliferating Blimp1− cells are the portion. In some embodiments, the proliferating non-stem cells are the portion. In some embodiments, the Blimp1⁺ cells are the portion. In some embodiments, the stem cells are the portion. In some embodiments, the asymmetrical division is into a proliferating cell and a differentiated cell. In some embodiments, the asymmetrical division is into a stem cell and a differentiated cell. In some embodiments, the asymmetrical division is into a stem cell and a sebocyte-like cell. In some embodiments, the differentiated cell is a sebocyte-like cell. In some embodiments, the asymmetrical division is into a stem cell and a sebocyte. In some embodiments, the differentiated cell is a sebocyte. In some embodiments, the asymmetrical division is into an outer layer cell and an inner core cell. In some embodiments, a portion of outer layer cells migrate to the inner core. In some embodiments, upon division into a differentiated cell the differentiated cell migrates to the inner core. In some embodiments, the organoid is characterized by outer layer cells that migrate to the inner core.

In some embodiments, an inner core cell is a differentiated cell. In some embodiments, an inner core cell is a sebocyte-like cell. In some embodiments, the inner core comprises differentiated cells. In some embodiments, the inner core comprises sebocyte-like cells. In some embodiments, the inner core is devoid of proliferating cells. In some embodiments, the inner core is substantially devoid of proliferating cells. In some embodiments, the inner core comprises lipid-producing cells. In some embodiments, the inner core cells are larger than the outer layer cells.

In some embodiments, the inner core comprises at least 5, 10, 15, 20, 25, 30, 35, 40, 45, 50, 55, 60, 65, 70, 75, 80, 85, 90, 95 or 100 cells. Each possibility represents a separate embodiment of the invention. In some embodiments, the inner core comprises at least 15 cells. In some embodiments, an organoid grown for between 10-14 days comprises an inner core comprising at least 15 cells. In some embodiments, the inner core comprises between 5-200, 5-150, 5-100, 5-90, 5-80, 5-70, 5-60, 5-50, 5-40, 10-200, 10-150, 10-100, 10-90, 10-80, 10-70, 10-60, 10-50, 10-40, 15-200, 15-150, 15-100, 15-90, 15-80, 15-70, 15-60, 15-50, 15-40, 20-200, 20-150, 20-100, 20-190, 20-80, 20-17, 20-16, 20-50, or 20-40 cells. Each possibility represents a separate embodiment of the invention. In some embodiments, the inner core comprises between 15-50 cells. In some embodiments, an organoid grown for between 10-14 days comprises an inner core comprising between 15-50 cells. In some embodiments, an organoid grown for between 7-9 days comprises an inner core comprising between 5-40 cells.

In some embodiments, the inner core comprises a diameter of at least 50, 75, 100, 125, 150, 175, 200, 225, 250, 275, 300, 325, 350, 375, or 400 μm. Each possibility represents a separate embodiment of the invention. In some embodiments, the inner core comprises a diameter of at least 1, 2, 3, 5, 10, 15, 20, 25, or 30 cells. Each possibility represents a separate embodiment of the invention. In some embodiments, inner core comprises a diameter of between 3-30, 3-25, 3-20, 3-15, 3-10, 3-5, 4-30, 4-25, 4-20, 4-15, 4-10, 5-30, 5-25, 5-20, 5-15 or 5-10 cells. Each possibility represents a separate embodiment of the invention. In some embodiments, the inner core comprises a diameter of between 4-10 cells. In some embodiments, an organoid grown for between 10-14 days comprises an inner core comprising between 4-10 cells. In some embodiments, an organoid grown for between 7-9 days comprises an inner core comprising between 2-8 cells.

In some embodiments, the cells of the inner core are large and diffuse. In some embodiments, the cells of the inner core comprise a diameter of at least 20, 25, 30, 35, 40, 45, 50, 55, 60, 65, 70 or 75 μm. Each possibility represents a separate embodiment of the invention. In some embodiments, the cells of the inner core comprise a diameter of about 36 um. In some embodiments, the cells of the inner core comprise an average diameter of 36 um. In some embodiments, the cells of the inner core comprise a diameter of between 20-75, 20-70, 20-65, 20-60, 20-55, 20-50, 20-45, 20-40, 25-75, 25-70, 25-65, 25-60, 25-55, 25-50, 25-45, 25-40, 30-75, 30-70, 30-65, 30-60, 30-55, 30-50, 30-45, 30-40, 35-75, 35-70, 35-65, 35-60, 35-55, 35-50, 35-45, 35-40, 40-75, 40-70, 40-65, 40-60, 40-55, or 40-50 um. Each possibility represents a separate embodiment of the invention. In some embodiments, the cells of the inner core comprise a diameter of between 20-50 um. In some embodiments, the cells of the inner core are substantially round. In some embodiments, the cells of the inner core comprise a volume of at least 30, 50, 70, 100, 130, 150, 170, 200, 250, 300, 350, 400, 450, 500, 600, 700, 800, 900, 1000, 1200, 1400, 1600, 1800, 2000, 2500, 3000, 3500, 4000, 4500, 5000 μm³. Each possibility represents a separate embodiment of the invention.

In some embodiments, the inner core comprises a Blimp1+ cell. In some embodiments, the inner core comprises Blimp1⁺ cells. In some embodiments, the inner core comprises Blimp1− cells. In some embodiments, the inner core comprises a Blimp1− cell. In some embodiments, the inner core comprises at least 1, 3, 5, 10, 15, 20, 25, 30, 35, 40, 45, or 50% Blimp1⁺ cells. Each possibility represents a separate embodiment of the invention. In some embodiments, the inner core comprises between 0.5-30, 0.5-25, 0.5-20, 0.5-15, 0.5-10, 0.5-7, 0.5-5, 1-30, 1-25, 1-20, 1-15, 1-10, 1-7, 1-5, 2-30, 2-25, 2-20, 2-15, 2-10, 2-7, 2-5, 3-30, 3-25, 3-20, 3-15, 3-10, 3-7, or 3-5% Blimp1⁺ cells. Each possibility represents a separate embodiment of the invention. In some embodiments, the inner core comprises between 1-10% Blimp1⁺ cells. In some embodiments, the inner core comprises a similar number (+/−10%) of Blimp1⁺ cells as compared to the inner core of the in vivo organ. In some embodiments, the inner core Blimp1⁺ cells are not stem cells. In some embodiments, the inner core Blimp1⁺ cells are terminally differentiated cells.

Analogously to a natural sebaceous gland, this organoid comprises Blimp1 expressing stem cells in its outer layer. These cells divide and produce both Blimp1+ stem cells and Blimp1− proliferating cells. These Blimp1− cells proliferate and can differentiated into sebocyte-like cells that are still Blimp1− and migrate into the inner mass. In the inner mass these cells develop like sebocytes and eventually re-express Blimp1. Re-expression of Blimp1 coincides roughly with production of sebum.

In some embodiments, the inner core comprises at least 10, 15, 20, 25, 30, 35, 40, 45, 50, 55, 60, 65, 70, 75, 80, 85, 90, 95, 97, 99 or 100% lipid-producing cells. Each possibility represents a separate embodiment of the invention. Lipid production can be examined by staining with Oil-red O (ORO) dye, or by other limit marking dyes, for example. In some embodiments, the sebocyte-like cells and/or lipid-producing cells stain positive for ORO dye. In some embodiments, the sebocyte-like cells and/or lipid-producing cells comprise a lipid composition similar to vivo sebocytes. In some embodiments, the sebocyte-like cells and/or lipid-producing cells secrete a lipid composition similar to vivo sebocytes. In some embodiments, the sebocyte-like cells and/or lipid-producing cells comprise a lipid composition similar to vivo SG. In some embodiments, the sebocyte-like cells and/or lipid-producing cells secrete a lipid composition similar to vivo SG.

In some embodiments, the sebocyte-like cells and/or lipid-producing cells express at least one sebocyte marker. In some embodiments, the marker is selected from the group consisting of androgen receptor (AR), fatty acid synthase (FASN), peroxisome proliferator-activated receptor beta (PPAR-B) and peroxisome proliferator-activated receptor gamma (PPAR-γ). In some embodiments, the marker is AR. In some embodiments, the marker is FASN. In some embodiments, the marker is PPAR-B. In some embodiments, the marker is PPAR-γ. The expression of a marker may be checked by immunoblotting, immunostaining, ELISA, FACS or any similar assay known in the art.

In some embodiments, the Blimp1 expressing cell is a eukaryotic cell. In some embodiments, the Blimp1 expressing cell is a mammalian cell. In some embodiments, the Blimp1 expressing cell is a murine cell. In some embodiments, the Blimp1 expressing cell is a human cell. In some embodiments, the Blimp1 expressing cell is a Blimp1 expressing skin cell. In some embodiments, the Blimp1 expressing cell is an epidermal cell. In some embodiments, the Blimp1 expressing cell is a keratinocyte. In some embodiments, the Blimp1 expressing cell is an epidermal keratinocyte. In some embodiments, the Blimp1 expressing cell is from a SG. In some embodiments, the Blimp1 expressing cell is a mitotic cell. In some embodiments, the Blimp1 expressing cell is an actively dividing cell. In some embodiments, the Blimp1 expressing cell is not a terminally differentiated cell. In some embodiments, the Blimp1 expressing cell is a stem cell. In some embodiments, the Blimp1 expressing cell is derived from a stem cell. In some embodiments, the Blimp1 expressing cell is a primary cell. In some embodiments, the Blimp1 expressing cell is derived from a primary cell.

As used herein, the terms “Blimp1 expressing cell” and “Blimp1+ cell” are synonymous and used interchangeably. In some embodiments, the Blimp1 expressing cell expresses integrin alpha 6 (a6). In some embodiments, the Blimp1 expressing cell does not express lymphocyte antigen 6 complex (SCA-1). In some embodiments, the Blimp1 expressing cell is a stem cell. In some embodiments, the Blimp1 expressing cell is a primary cell. In some embodiments, the primary cell is a skin cell. In some embodiments, the primary cell is an epidermal cell. In some embodiments, the epidermal cell is a keratinocyte.

In some embodiments, the growth matrix is a 3D growth matrix. In some embodiments, the growth matrix is Matrigel. In some embodiments, the growth matrix is a 3D scaffold. In some embodiments, the Blimp1 expressing cell is cultured alone. In some embodiments, only Blimp1 expressing cells are cultured. In some embodiments, only Blimp1 expressing skin cells are cultured. In some embodiments, only Blimp1 expressing skin stem cells are cultured. In some embodiments, the Blimp1 expressing cell is cocultured with at least one other cell. In some embodiments, the method further comprises coculturing another cell in the 3D growth matrix. In some embodiments, the method further comprises coculturing another cell with the Blimp1 expressing skin stem cell. In some embodiments, the Blimp1 expressing cell is cocultured with at least one other Blimp1 expressing cell. In some embodiments, the other cell is a Blimp1 expressing skin cell. In some embodiments, the other cell is a Blimp1 expressing mitotic skin cell. In some embodiments, the other cell is a Thy1.2 expressing cell. In some embodiments, the method further comprises providing the Blimp1 expressing cell. In some embodiments, the method further comprises providing the other cell. In some embodiments, the providing comprises FACS sorting. In some embodiments, the FACS sorting is FACS sorting of skin cells.

In some embodiments, the Thy1.2 expressing cell highly expresses Thy1.2. In some embodiments, the Thy1.2 expressing cell is a stem cell. In some embodiments, the Thy1.2 expressing cell is a skin cell. In some embodiments, the Thy1.2 expressing cell is an epidermal cell. In some embodiments, the Thy1.2 expressing cell is an interfollicular epidermis cell. In some embodiments, the Thy1.2 expressing cell is a keratinocyte. In some embodiments, the Thy1.2 expressing cell is an interfollicular epidermis stem cell.

In some embodiments, the culturing comprises addition of media. In some embodiments, the cell is cultured in media. In some embodiments, the culturing is in media. In some embodiments, the growth matrix comprises media. In some embodiments, the media is stem cell media. In some embodiments, the media is skin stem cell media. In some embodiments, the media is hair follicle stem cell media. In some embodiments, the media comprises between 20-80, 20-70, 20-60, 20-50, 30-80, 30-70, 30-60, 30-50, 40-80, 40-70, 40-60, 40-50, 45-55, 46-54, 47-53, 48-52, or 49-51 mM calcium (Ca2+). Each possibility represents a separate embodiment of the invention. In some embodiments, the media comprises between 45-55 mM Ca2+. In some embodiments, the media comprises about 50 mM Ca2+. In some embodiments, the media comprises 50 mM Ca2+.

In some embodiments, the media comprises at least one growth factor. In some embodiments, the growth factor is selected from the group consisting of: epidermal growth factor (EGF), fibroblast growth factor (FGF), Noggin and R-spondin. In some embodiments, the growth factor is EGF. In some embodiments, the growth factor is FGF. In some embodiments, the growth factor is Noggin. In some embodiments, the growth factor is R-spondin. In some embodiments, the media comprises EGF, FGF, Noggin and R-spondin. In some embodiments, the media comprises an agent that inhibits cell-cell interaction-dependent anoikis. In some embodiments, when the Blimp1 expressing primary skin cell in not precultured on feeder cells the culturing in a 3D growth matrix is in the presence of an agent that inhibits cell-cell interaction-dependent anoikis. In some embodiments, the agent that inhibits cell-cell interaction-dependent anoikis is a ROCK inhibitor. In some embodiments, the media comprises a ROCK inhibitor. In some embodiments, the ROCK inhibitor is Y27632.

In some embodiments, the method of the invention further comprises preculturing a Blimp1 expressing cell on a feeder cell or feeder cell layer. In some embodiments, the culturing on a feeder cell layer is for at least 1, 2, 3, 4, 5, 6, 7, 8, 9 or 10 passages. Each possibility represents a separate embodiment of the invention. In some embodiments, the culturing on a feeder layer is for at least 4 passages. In some embodiments, the culturing on a feeder layer is for at least 8 passages. In some embodiments, the culturing on a feeder cell layer is for between 6-12, 6-11, 6-10, 7-12, 7-11, 7-10, 8-12, 8-11 or 8-10 passages. Each possibility represents a separate embodiment of the invention. In some embodiments, the culturing on a feeder cell layer is for between 8-10 passages. some embodiments, after the culturing on a feeder layer at least one Blimp1 expressed cell is selected and/or transferred to the growth matrix. In some embodiments, only a mitotic Blimp1 expressing cell is transferred. In some embodiments, the number of passages is sufficient such that the culture is devoid, or substantially devoid, of terminally differentiated cells. In some embodiments, the culture is devoid of terminally differentiated SG and/or IFE cells. In some embodiments, the feeder cells are fibroblasts. In some embodiments, the feeder cells are mammalian cells. In some embodiments, the feeder cells are murine cells. In some embodiments, the feeder cells are human cells. In some embodiments, the feeder cells are J2 feeder cells. In some embodiments, the feeder cells are 3T3-J2 feeder cells. In some embodiments, the feeder cells are irradiated or not irradiated.

In some embodiments, the time sufficient for production of the organoid is at least 4, 5, 6, 7, 8, 9, 10, 11, or 12 days. Each possibility represents a separate embodiment of the invention. In some embodiments, the time sufficient for production of the organoid is at least 8 days. In some embodiments, the time sufficient for production of the organoid is at least 12 days. In some embodiments, the time sufficient for production of the organoid is between 6-12, 6-11, 6-10, 7-12, 8-12, 9-12, 7-11, 8-11, 9-11, 7-10, 8-10, or 9-10 day. Each possibility represents a separate embodiment of the invention. In some embodiments, the time sufficient for production of the organoid is between 8-10 days. The production of the organoid can be monitored by any means which would inform the producer that the organoid had be successfully produced. This would include, but not be limited to, microscopy to check for the morphology or organization of the cells/organoid, immunostaining or immunoblotting for markers of the organoid's creation, such as those markers described herein, and testing of the lipid secretion from the organoid. It will be understood that not all of these identifying features may start of the same day after culturing, but rather the performer of the method can halt culturing at a time that suits their particular use of the organoid. In some embodiments, the culture is for a time sufficient to produce all the markers of the organoid described herein. In some embodiments, the culture is for a time sufficient to produce at least one of the markers of the organoid described herein.

In some embodiments, the organoid is a model of a naturally occurring SG. In some embodiments, the organoid is a model of a healthy naturally occurring SG. In some embodiments, the organoid is a model of a hyper-proliferative organoid. In some embodiments, the organoid is a model of an acne producing organoid. In some embodiments, acne is acne vulgaris. In some embodiments, the organoid is a model of a pathogenic organoid. In some embodiments, the organoid is contacted with at least one of dihydrotestosterone androgen (DHT), PPAR-γ BRL-49653 activator (BRL) and linoleic acid (LIN). In some embodiments, the organoid is contacted with DHT. In some embodiments, the organoid is contacted with BRL. In some embodiments, the organoid is contacted with LIN. In some embodiments, the organoid is contacted with DHT, BRL and LIN.

By another aspect there is provided a composition comprising the organoid of the invention and a growth matrix. In some embodiments, the growth matrix is a 3D growth matrix. In some embodiments, the growth matrix is Matrigel. In some embodiments, the composition is ex vivo. In some embodiments, the composition is in vivo.

By another aspect, there is provided a method of testing a skin drug, the method comprising contacting an organoid of the invention with the skin drug. In some embodiments, the testing is ex vivo. In some embodiments, the testing is in vivo. In some embodiments, the organoid is in a model organism while testing. In some embodiments, the model organism is murine. In some embodiments, the model organism is a mammal. In some embodiments, the model organism is a monkey.

In some embodiments, the contacting comprises addition of the drug to the organoid's growth media. In some embodiments, the contacting comprises addition of the drug to the growth matrix. In some embodiments, the contacting comprises topical contact with the organoid. In some embodiments, the contacting comprises injection into the organoid.

In some embodiments, the drug is a skin drug. In some embodiments, the drug is an acne drug. In some embodiments, acne is acne vulgaris. In some embodiments, the drug is a cancer drug. In some embodiments, the cancer is cancer of the SB. In some embodiments, the cancer is sebaceous gland carcinoma. In some embodiments, the drug treats a sebaceous gland cyst. In some embodiments, the drug treats a disease or pathology of the SB. In some embodiments, the disease or pathology of the SB is selected from seborrhea, seborrheic dermatitis, acne, androgenetic alopecia, rosacea and cancer. In some embodiments, the drug targets a cell of the SB. In some embodiments, the drug targets the SB. In some embodiments, the testing is testing the side effects of the drug on the SB. In some embodiments, the drug does not target the SB, and the testing is testing side effects of the drug on the SB.

In some embodiments, the method further comprises contacting the organoid with at least one of dihydrotestosterone androgen (DHT), PPAR-γ BRL-49653 activator (BRL) and linoleic acid (LIN). In some embodiments, the method further comprises contacting the organoid with DHT. In some embodiments, the method further comprises contacting the organoid with BRL. In some embodiments, the method further comprises contacting the organoid with LIN. In some embodiments, the method further comprises contacting the organoid with DHT, BRL and LIN.

By another aspect, there is provided a method of modeling acne in a SG, comprising providing an SG-like organoid of the invention and contacting the organoid with at least one of dihydrotestosterone androgen (DHT), PPAR-γ BRL-49653 activator (BRL) and linoleic acid (LIN), thereby modeling acne in a SG. In some embodiments, the method is for producing an acne-like SG organoid. In some embodiments, the method is for producing a hyperproliferative SG organoid. In some embodiments, the method is for producing a pathological SG organoid. In some embodiments, the pathology is acne. In some embodiments, the acne is acne-vulgaris.

By another aspect, there is provided a method of treating acne in a subject in need thereof, the method comprising decreasing c-Myc expression, function or both in a SG of the subject, thereby treating acne in the subject.

By another aspect, there is provided a pharmaceutical composition comprising a c-Myc inhibitor. In some embodiments, the composition further comprises a pharmaceutically acceptable carrier, excipient or adjuvant. In some embodiments, the pharmaceutical composition is for use in treating acne. In some embodiments, the pharmaceutical composition is configured to topical or dermatological administration. In some embodiments, the pharmaceutical composition is in the form of a gel, paste, foam or cream.

In some embodiments, decreasing c-Myc expression comprises decreasing c-Myc mRNA, protein or both. In some embodiments, decreasing c-Myc expression comprises decreasing c-Myc mRNA. In some embodiments, decreasing c-Myc expression comprises decreasing c-Myc protein. In some embodiments, decreasing c-Myc function comprises administering a c-Myc inhibitor. In some embodiments, the decreasing is in the lipid-producing cells of the SG. In some embodiments, the decreasing is in the inner cells of the SG. In some embodiments, the decreasing is in the sebocytes of the SG. In some embodiments, the decreasing is in the outer cells of the SG.

In some embodiments, the decreasing comprises contacting the SG with a c-Myc inhibitor. In some embodiments, contacting with a c-Myc inhibitor comprises contacting with a pharmaceutical composition comprising a c-Myc inhibitor. In some embodiments, the contacting is with the skin of the subject. In some embodiments, the contacting is with an area of the skin of the subject comprising acne. In some embodiments, the treating is contacting an area of skin of the subject comprising acne.

In some embodiments, the c-Myc inhibitor is 10058-F4. As used here in 10058-F4 refers to 5-[(4-Ethylphenyl)methylene]-2-thioxo-4-thiazolidinone. In some embodiments, a therapeutically effective dose of the c-Myc inhibitor is contacted. In some embodiments, the c-Myc inhibitor is in a pharmaceutical composition. In some embodiments, the pharmaceutical composition comprises a pharmaceutically acceptable carrier, adjuvant or excipient. In some embodiments, the pharmaceutical composition is configured for topical administration. In some embodiments, the pharmaceutical composition is in the form of a gel, paste, foam or cream.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the terms “administering,” “administration,” and like terms refer to any method which, in sound medical practice, delivers a composition containing an active agent to a subject in such a manner as to provide a therapeutic effect. One aspect of the present subject matter provides for topical administration of a therapeutically effective amount of a composition of the present subject matter to a patient in need thereof. Other suitable routes of administration can include parenteral, subcutaneous, intravenous, oral, dermal, subdermal, intramuscular, or intraperitoneal.

The dosage administered will be dependent upon the age, health, and weight of the recipient, kind of concurrent treatment, if any, frequency of treatment, and the nature of the effect desired.

The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods:

Mice: All animal studies were approved by the Committee on the Ethics of Animal Experiments of the Technion. B6.Cg-Tg(Prdm1-EYFP)1Mnz/J mice were purchased from Jackson. Brainbow1.0, Brainbow 2.1, B6.129P2-Gt(ROSA)26Sortm1(CAG Brainbow2.1)C1 FVB/N-Tg(Thylcre)1V1n/fIg and B6; SJL-Tg(Thy1-cre/ERT2, −EYFP)VGfng/J mice were purchased from Jackson. For lineage tracing Tamoxifen (TMX) was dissolved in DMSO (30 mg/ml) and diluted in PBS (1.5 mg/ml). 200 μl were injected subcutaneous or TMX dissolved in ethanol (150 mg/ml) was applied topically on shaved and depilated mouse dorsal skin for five consecutive days. For 5-Bromo-2′-deoxyuridine (BrdU) (GE Healthcare) pulse-chase experiments mice were injected subcutaneously with 50 μg/g BrdU (Sigma-Aldrich) and animals were sacrificed thereafter, either directly or following a chase period.

Flow cytometry: α6+ScaI−Blimp−YFP+ cells isolation was performed by fluorescence-activated cell sorting (FACS) with Blimp1−YFP, Integrin α6 and Sca1 antibodies. Cells were grown as previously described (Fuchs et al., 2013). To visualize Blimp−YFP+sorted cells, imaging flow cytometry (IFC) was performed using the Amnis ImageStreamX Mark II Flow cytometer.

Fluorescence-activated cell sorting (FACS) was performed with Blimp1−YFP, Lgr6-GFP, CD34, Integrin α6, Integrin β1, Thy1.2 and Sca1 antibodies.

Cell culture: α6+ScaI−Blimp−YFP+ cells were cultured in hair follicle stem cell (HFSC) media on sustaining J2 feeder cells for 4-6 passages until stable colonies were formed, followed by culturing without the feeders. Serum was chelated for a calcium concentration of 50 μM.

For organoid generation a total of ˜1000 cells were mixed with 50 μl of Matrigel (BD Bioscience) and plated in 24-well plates. After polymerization of Matrigel, 500 μl of HFSC media containing growth factors; 20 ng ml-1 hFGF (Peprotech), 40 ng ml-1 mEGF (Peprotech), 500 ng ml-1 hR-spondin 1, 100 ng ml-1 mNoggin (Peprotech) and B-27 Supplement (50×) (Invitrogen)) was added.

Organoid treatment with cMyc inhibitor 10058-F4 (50 Tocris Bioscience) or DMSO (as control) was performed for 4 days and renewed upon media change. Organoids were fixed in 4% PFA for 1 hour for immunofluorescence analysis or harvested for RNA extraction.

For 5-Bromo-2′-deoxyuridine (BrdU) (GE Healthcare) pulse-chase experiments, 6 days old organoids were treated with BrdU (1:1000) for 8 hours and organoids were harvested for immunofluorescence analysis following a chase period of 24, 48 or 72 hours.

α6+CD34⁺ and α6+CD34⁻Thy1.2^{high} cells were isolated and cultured in hair follicle stem cell (HFSC) media on sustaining J2 feeder cells as described previously (Fuchs et al., 2013). Serum was chelated for a calcium concentration of 50 μM.

Histology and Immunofluorescence: For whole mount preparation, tail skin samples were treated with 5 mM EDTA for 4 hrs at 37° C. to separate skin epithelium from dermis and fixed in 4% formaldehyde for 2 hrs at room temperature. Dorsal skin samples were treated with 5 mM EDTA for 6 hrs at 37° C. to separate skin epithelium from dermis and fixed in 4% formaldehyde for 1 hr at room temperature.

Organoids were fixed in 4% PFA for 1 hour for immunofluorescence analysis.

Samples were blocked for 2 hrs in blocking buffer consisting of 10% goat serum, 2% BSA and 1% Triton-X. Primary antibodies were diluted in blocking buffer and samples were incubated overnight at 4° C. Samples were washed at least 3 times with PBS. Secondary antibodies were incubated for 1 hr at room temperature followed by 4 washes with PBS.

The following primary antibodies were used: Ki67 (Rabbit, 1:100, Abcam; Rat, 1:100, eBioscience), MCM2 (Rabbit, 1:500, Abcam), BrdU (mouse, 1:200, Santa Cruz), cMyc (mouse, 1:100, Santa Cruz), Phalloidin (1:250, Life Technologies), K15 (Mouse, 1:100, Thermo, Chicken, 1:1000, Abcam), K5 (Rabbit, 1:500, Abcam).

Antibody staining was visualized using secondary antibodies conjugated to Alexa Fluor-488, Alexa Fluor-546, and Alexa Fluor-633. Analysis was performed on Zeiss LSM 880 confocal microscope. Oil-red-O (ORO) staining was performed by incubating samples in 0.18% ORO for 20 min and washing with PBS.

Tailskin samples were treated with 5 mM EDTA for 4 hrs at 37° C. to separate skin epithelium from dermis and fixed in 4% formaldehyde for 2 hrs at room temperature. Dorsalskin samples were treated with 5 mM EDTA for 6 hrs at 37° C. to separate skin epithelium from dermis and fixed in 4% formaldehyde for 1 hr at room temperature. Skins were embedded in OCT, frozen, sectioned and fixed in 4% formaldehyde.

Epitope retrieval was performed by incubation of wholemount samples in citrate buffer (pH=6) at 95° C. for 7 min. Samples were blocked for 2 hrs in blocking buffer consisting of 10% goat serum, 2% BSA and 1% Triton-X. Primary antibodies were diluted in blocking buffer and tissue or sections were incubated overnight at 4° C. Wholemounts or sections were washed at least 3 times with PBS. Secondary antibodies were incubated for 1 hr at room temperature followed by 4 washes with PBS.

The following primary antibodies were used: Ki67 (Rabbit, 1:100, Abcam; Rat, 1:100, eBioscience), CD34 (Rat, 1:100, eBioscience), CD90.2-PE conjugated (Rat, 1:100, BD Bioscience), BrdU (mouse, 1:200, Santa Cruz), Lrig1-488 conjugated (Goat, 1:50, R&D systems), Axin2 (Rabbit, 1:100, Abcam), CD104 (Rat, 1:100, BD Pharmingen). Antibody staining was visualized using secondary antibodies conjugated to Alexa Fluor-488, Alexa Fluor-546, and Alexa Fluor-633. Analysis was performed on Zeiss LSM 880 confocal microscope. Oil-red-O (ORO) staining was performed by incubating samples in 0.18% ORO for 20 min and washing with PBS.

RNA extraction, reverse transcription and real-time PCR: RNA was isolated using Trizol (Sigma) and up to 2 μg of RNA were subjected to cDNA synthesis (Applied Biosystems). Real time PCR was carried out using the PerfeCTa SYBR Green FastMix (Quanta), with the following gene-specific primers: RpLO: GCGACCTGGAAGTCCAACTA (SEQ ID NO: 1) and ATCTGCTTGGAGCCCACAT (SEQ ID NO: 2); AR: AGAATCCCACATCCTGCTCAA (SEQ ID NO: 3) and AAGTCCACGCTCACCATATGG (SEQ ID NO: 4); PPAR-gamma: GATGGAAGACCACTCGCATT (SEQ ID NO: 5) and AACCATTGGGTCAGCTCTTG (SEQ ID NO: 6); PPAR-beta: TTCCTTCCAGCAGCTGTG (SEQ ID NO: 7) and TCGCACGCGTGGACC (SEQ ID NO: 8); FASN: CCCTTGATGAAGAGGGATCA (SEQ ID NO: 9) and ACTCCACAGGTGGGAACAAG (SEQ ID NO: 10). Amplicon levels were analyzed in triplicate and quantitated relative to a standard curve comprising cDNA, and values normalized to levels of the housekeeping gene (Rpl0). Reactions were: 3 min at 95° C., then 40 cycles of 10 sec at 95° C. and 30 sec at 60° C. with addition of melt curve step: 10 sec at 95° C., and increments of 0.5° C. every 5 sec between 65° C. and 95° C.

Lipidomics sample preparation: Solvents for sample preparation, as well as for chromatography (LC/MS grade acetonitrile, isopropanol, methanol, and water, HPLC grade chloroform) were purchased from Bio-Lab, Inc. Solvent composition is provided as volume-to-volume ratio. Lipids were extracted with the 2:1 chloroform:methanol solution according to the Folch method31. Phase separation in the extract was achieved by the addition of equivalent water volume and centrifugation at 2000 g for 5 min. The upper and middle phases were removed by aspiration, and the lower phase was dried in the nitrogen flow. Lipid extract was redissolved in 1:1 isopropanol:methanol and stored for analysis.

LC/MS data acquisition: For the reversed-phase chromatographic separation, Phenomenex Kinetex C-18-XB 150 mm column (3 mm i.d., 2 μm beads) was used. The column compartment of HPLC was maintained at 65° C., sample vials at 30° C. Mobile phase was composed of two solvent mixes as follows: A −60:40 acetonitrile:water mix having 10 mM ammonium formate (HPLC grade, Sigma), B −90:8:2 acetonitrile:isopropanol:water with 10 mM ammonium formate. For the solvent B, ammonium formate was dissolved in corresponding water volume before mixing with the organic solvents. The buffer loop in the autosampler was filled with 1:1 isopropanol:methanol. Mobile phase flow was 0.5 mL/min. Mobile phase composition was changing according to the following program with linear gradients: 0 min-15% B; 2 min-30% B; 2.5 min-48% B; 11 min-82% B; 11.5 min-99% B, maintained until 14 min, and then switched back to 15% B and equilibrated for 2.5 min. Chromatographic eluent was analyzed by ESI-MS Thermo Orbitrap Q Exactive mass spectrometer. Ion source supplied 50 AU of sheath gas flow and 20 AU of auxiliary gas flow. Auxiliary gas and the ion transfer capillary were heated to 350° C. S-Lens RF level was 80 AU. Analysis was performed in polarity switching mode with the negative mode voltage −3.5 kV, and the positive mode voltage +3.3 kV. Automatic gain control was tuned to 106, scanning range was from 120 to 1800 m/z with mass resolution 70,000 in both polarity modes. Tandem mass spectra for the corresponding samples were acquired in the separate technical repeats with the following parameters of data-dependent acquisition: isolation width 1.4 Da, maximum ion accumulation time prior to fragmentation 600 ms, normalized collision energy 30 AU, minimum AGC target 104, fixed first mass 50 Da, three fragmentation spectra per one MS1 scan, multiple-charge ions exclusion, resolution 17,500 for MS1 and MS2 scans, no polarity switching.

Untargeted Lipidomics analysis: According to the LC/MS method, two primary types of ions were detected in both polarity modes: protonated analytes and NH4+ adducts in the positive mode; deprotonated analytes and HCOO− adducts in the negative mode. Peak peaking and intensity calling was performed in XCMS39, 40Stable reproducibility of chromatograms allowed not to use peak alignment. A custom-made R script was used to identify M+0 peaks. For further analysis, only M+0 peaks of lipid compounds were considered, with the intensity not less than 5×10\hat{ }5 in at least one of samples. For lipid mass list, lipids from Lipid Mass Structure Database41 were used together with a list of compounds created by a combination of hydrophilic “head” structures with even-numbered fatty acids. Selected groups of peaks and their natural carbon mass-isotopomer distributions were verified manually in Maven software. Intensities in each sample were log-transformed and converted to z-scores. Additionally, to compare intensity profiles between compounds, second normalization to z-scores was applied to each row.

Principle component analysis was performed on normalized dataset. Together, first four principal components represented 81% of variance. Space of these four principal components, each representing more than 10% of total variance, was used for clustering of peak profiles. Clustering was performed with k-means algorithm for eight centers. Resulted clusters were attributed to specific groups of samples according to the maximal intensity in the averaged profile.

Wound repair assay: 50-day old mice received subcutaneous injections of TMX for 5 days prior to wound excision. In all wound repair experiments mice were sedated with isoflurane. Mice were shaved treated topically with a depilation cream. Full-thickness excision wounds (1 cm2) were generated on the dorsal skins and monitored for wound coverage in the following days. At the desired time post wounding, mice were euthanized with CO2 and the wounded skins were harvested and prepared for wholemounts as described above for immunofluorescence analysis.

RNA-Seq: α6⁺CD34⁺Sca1⁻Thy⁻ (CD34⁺) and α6⁺CD34⁻Sca1⁻Thy1.2^{high} were sorted into TRIzol-LS. Total RNA was isolated for polyA purification and cDNA library construction using the TruSeq v1.5 kit (Illumina). cDNA was barcoded using the Illumina Multiplexing Sample Preparation Oligonucleotide Kit and analyzed on two lanes of an Illumina HiSeq 2000 in a 100 bp single-end sequencing run. The resulting sequence reads were aligned to the reference genome (mm9) using Tophat default settings. Gene expression was estimated using the mm9 ncbi gene model using the Cufflinks software. Downstream analysis was performed using R Bioconductor.

### Example 1: Single Blimp1⁺ Cells Generate a Sebaceous Gland-Like Structure Ex Vivo

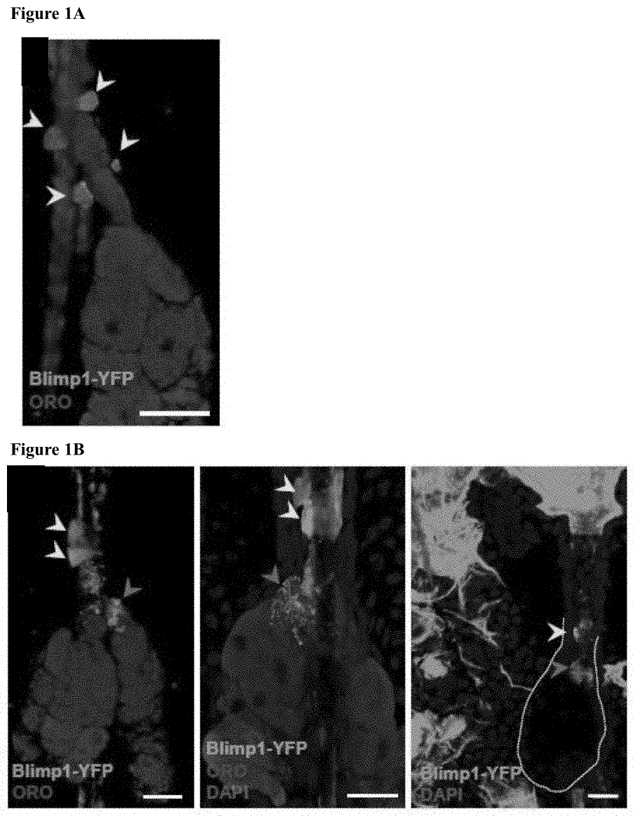

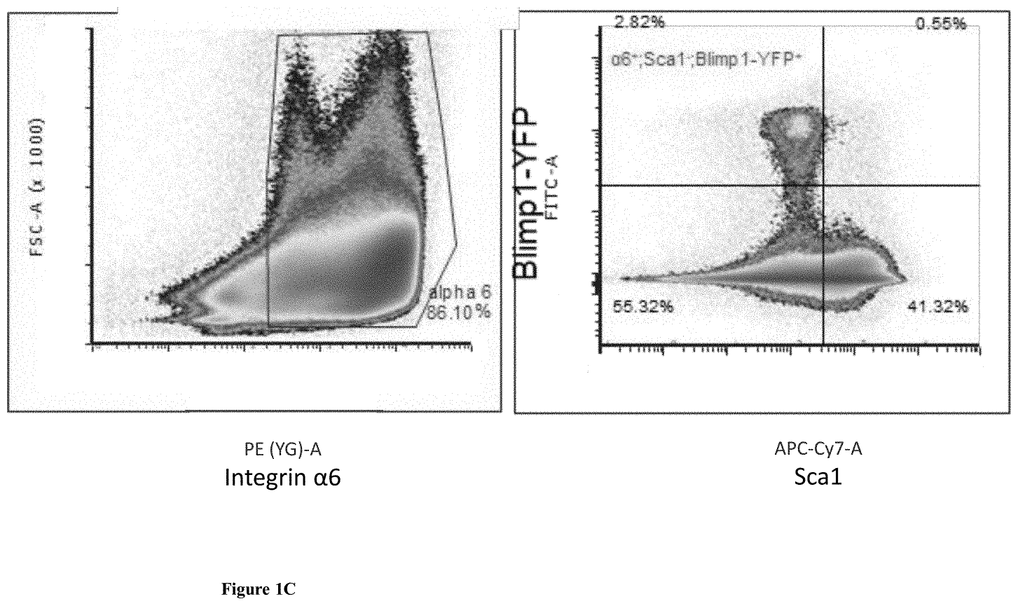

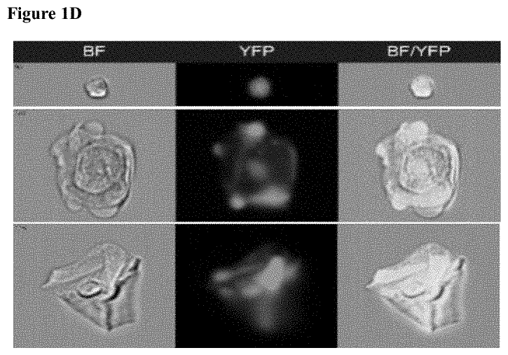

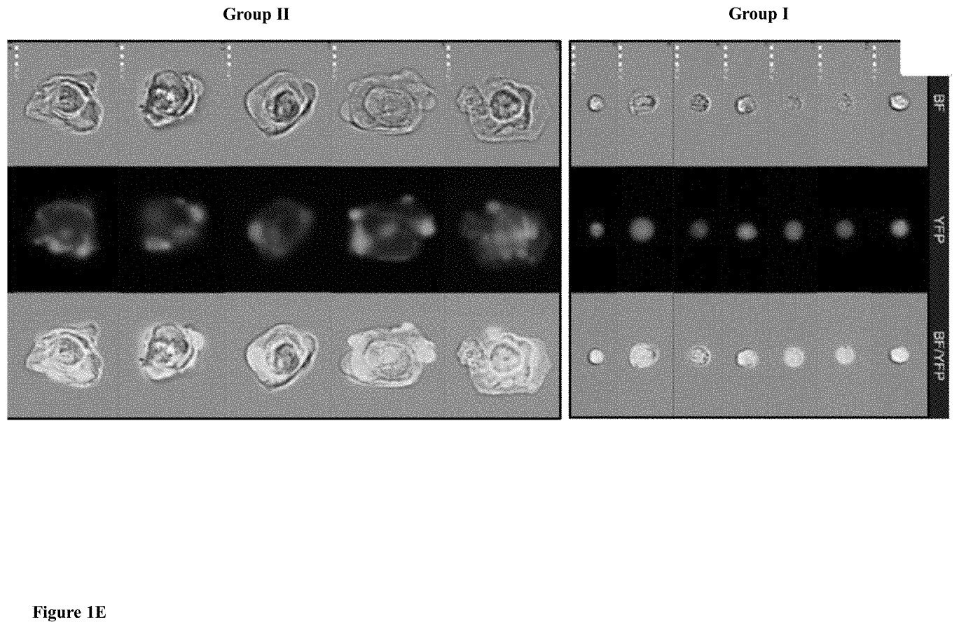

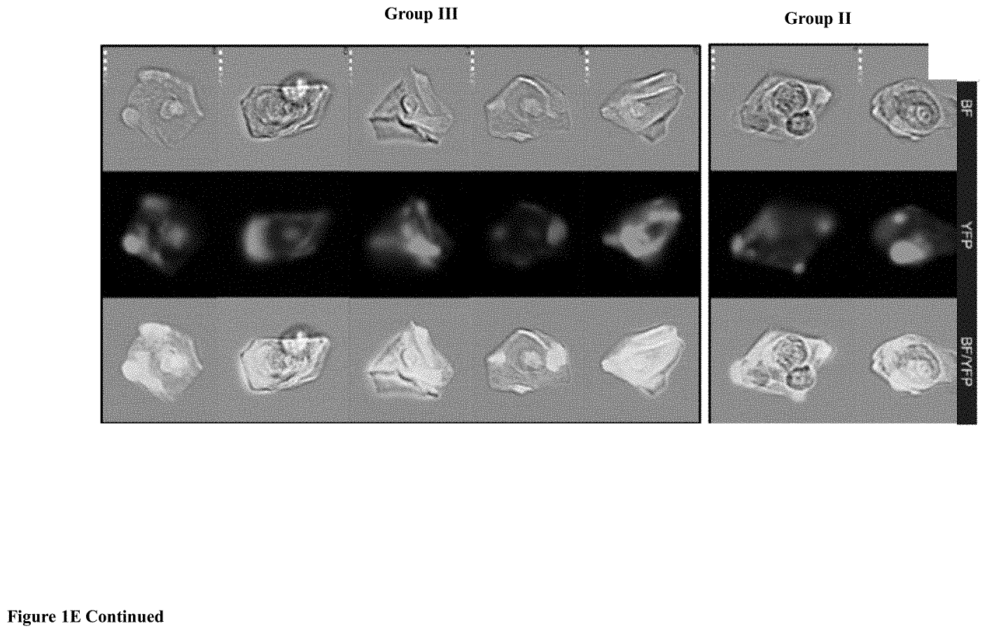

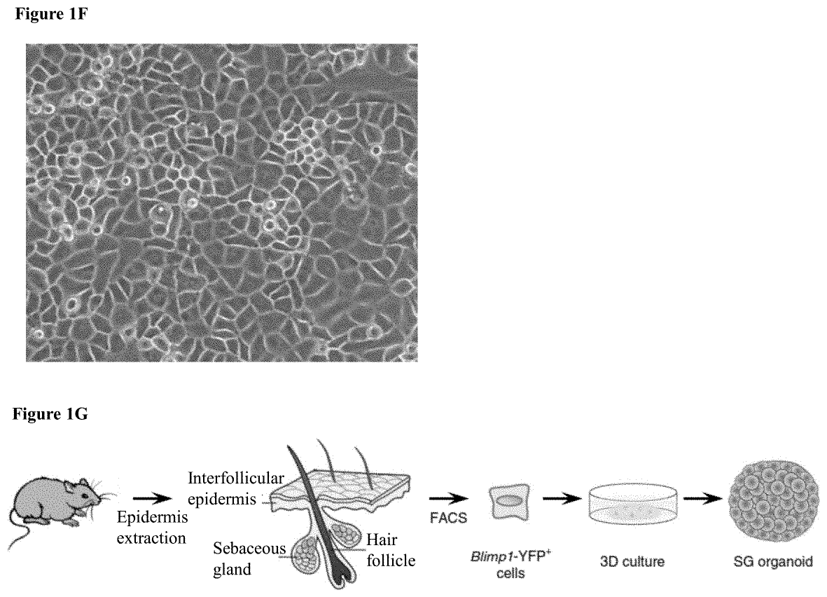

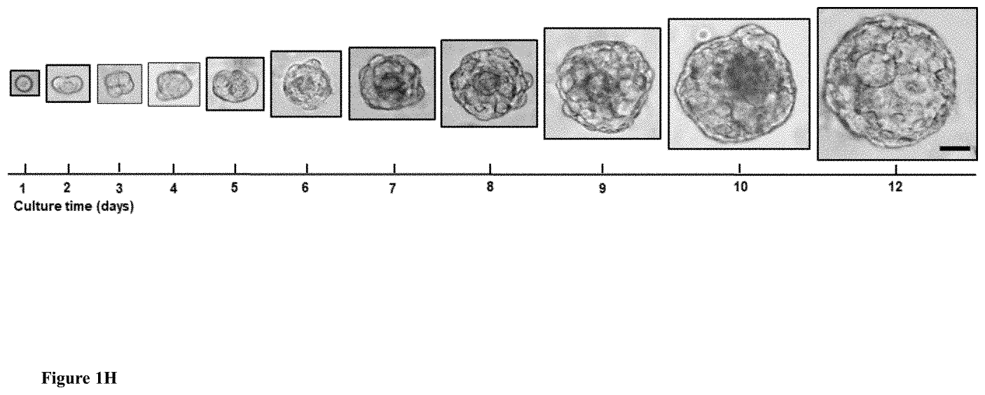

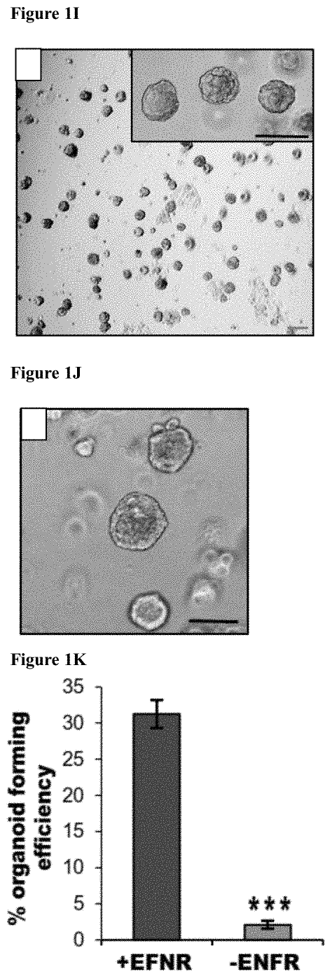

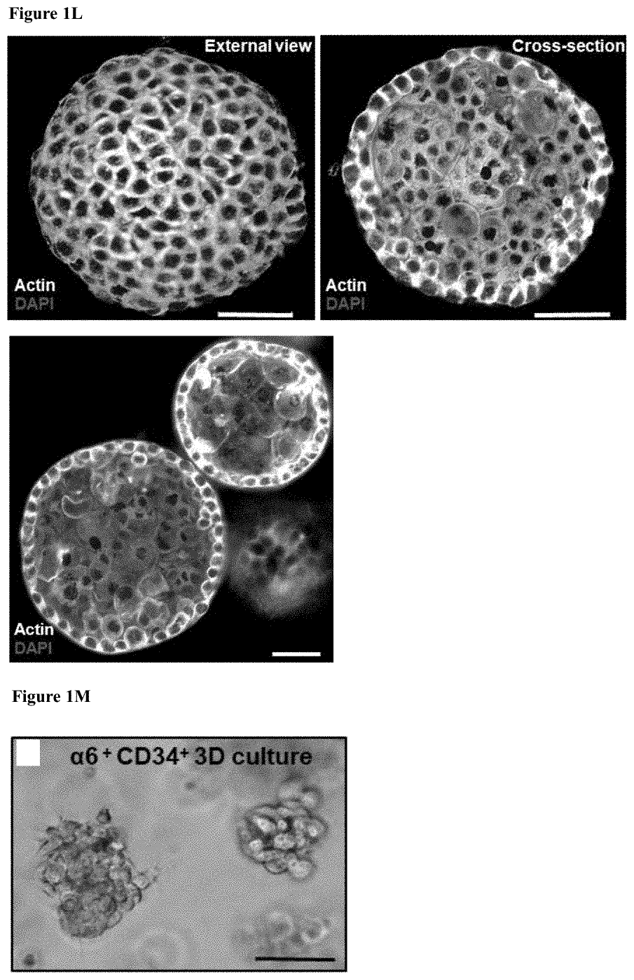

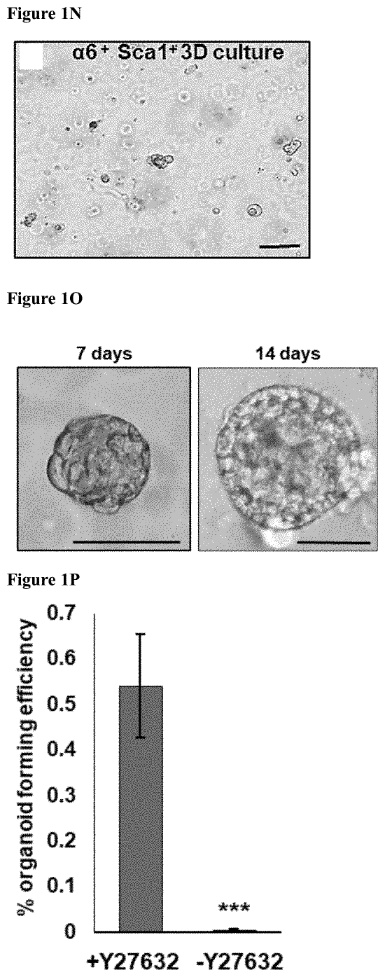

To investigate the function of Blimp1⁺ cells in the skin Blimp1−YFP reporter mice (B6.Cg-Tg(Prdm1-EYFP)1Mnz/J) were employed. By performing confocal Z-stack analysis it was found that Blimp 1 is expressed in cells at the base of the SG, as well as in differentiated SG cells and IFE granular cells (FIG. 1A-B). These findings suggest that Blimp1 does not mark a distinct cell population but is expressed in SG progenitors, mature sebocytes and differentiated IFE cells, as had been recently reported.

Next, fluorescence-activated cell sorting (FACS) was performed employing Blimp1-YFP mice and antibodies for integrin α6 (epidermal keratinocytes) and ScaI (IFE and infundibulum cells). Skin from 8-week old mice in second telogen were harvested, from which α6+; ScaI−; Blimp1−YFP+ cells were isolated (FIG. 1C). Additionally, the morphology of Blimp1−YFP+ cells could be examined by performing flow cytometry coupled with high-resolution microscopy (ImageStream), verifying that these cells highly express YFP (FIG. 1D-E). Furthermore, using this approach, and in accordance with previous findings, there was observed three morphologically distinct groups: (I) small cells with high circularity, (II) large cells resembling mature sebocytes and (III) differentiated IFE keratinocytes (FIG. 1E). α6+; ScaI−; Blimp1−YFP+ (Blimp1−YFP+) cells were expanded on J2 feeder cells for several passages in 2D culture (FIG. 1F), ensuring post-mitotic/terminally differentiated SG/IFE cells were completely excluded. Next, it was examined whether providing a basement membrane-like extracellular 3D environment such as Matrigel, which has been successfully utilized in several epithelial stem cell platforms, would enable establishment of SG organoids (FIG. 1G). α6+; ScaI−; Blimp1−YFP+ cells were imbedded in Matrigel and cultured in a custom-made epidermal media such as is described in Nowak and Fuchs, 2009, Isolation and Culture of Epithelial Stem Cells, Methods Mol Biol. 2009; 482:215-32, herein incorporated in reference in its entirety. Since epithelial cells are extremely sensitive to calcium, bovine serum was chelated, and cells were grown in 50 mM of Ca2+. Additionally, given that several key signaling cascades are known to regulate the function and expansion of epithelial cells in the pilosebaceous units the culture media was supplemented with EGF, FGF, Noggin and R-spondin (EFNR). Within one day, it was clear that α6+; ScaI−; Blimp1−YFP+ cells had started expanding, doubling every 24 hours (FIG. 1H). Strikingly, within twelve days, seeded single isolated Blimp1⁺ cells could generate large spheroid structures, which highly resembled the morphology of a SG in vivo (FIG. 1H). Additionally, single Blimp 1+ cells could generate a large number of organoids at a high efficiency (32% of plated cells) and could be passaged for extended periods of time (˜40 passages) (FIG. 1I). In contrast, when α6+; ScaI−; Blimp1−YFP+ cells were grown without EFNR factors, growth was significantly impaired and only a very limited number of organoids were generated (FIG. 1J-K).

Examining the morphology of these organoids revealed a very defined ring of compact cells at the outer layer, as well as larger cells of varying sizes in the inner mass that appeared reminiscent of differentiated sebocytes (FIG. 1L). However, when α6+; CD34+ HFSCs were grown in similar conditions, the formation of these structures was not observed. Such cultures resulted in non-organized spheroid clusters highly similar to those recently reported by others (Chacon-Martinez, C. A., Klose, M., Niemann, C., Glauche, I. & Wickstrom, S. A. Hair follicle stem cell cultures reveal self-organizing plasticity of stem cells and their progeny. EMBO J. 36, 151-164 (2017)) (FIG. 1M). Additionally, under these condition α6+; ScaI+ IFE/IFD cells did not give rise to any defined cellular structure (FIG. 1N).

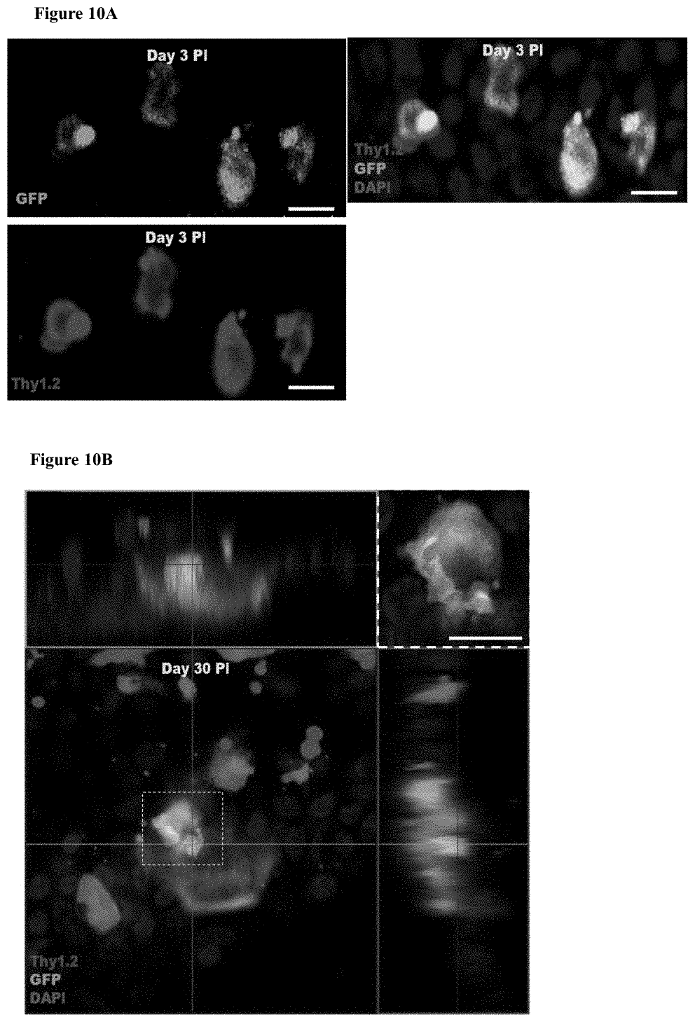

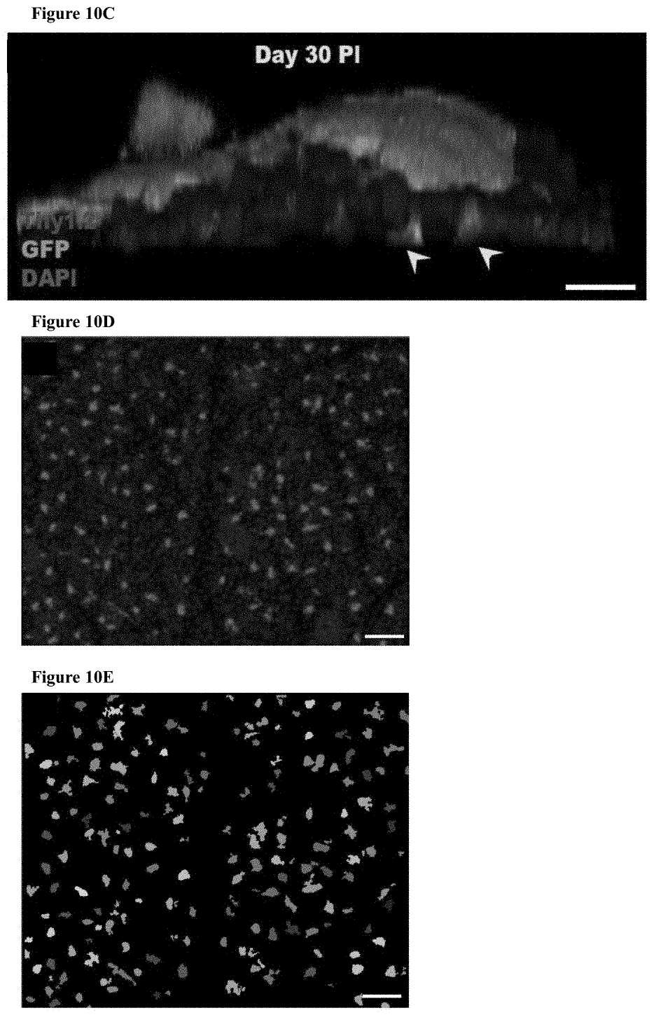

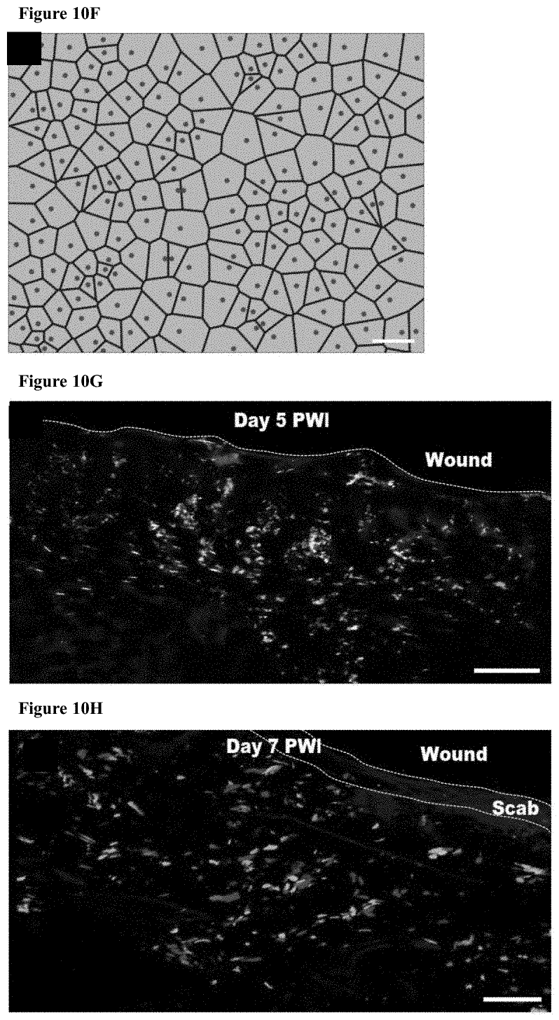

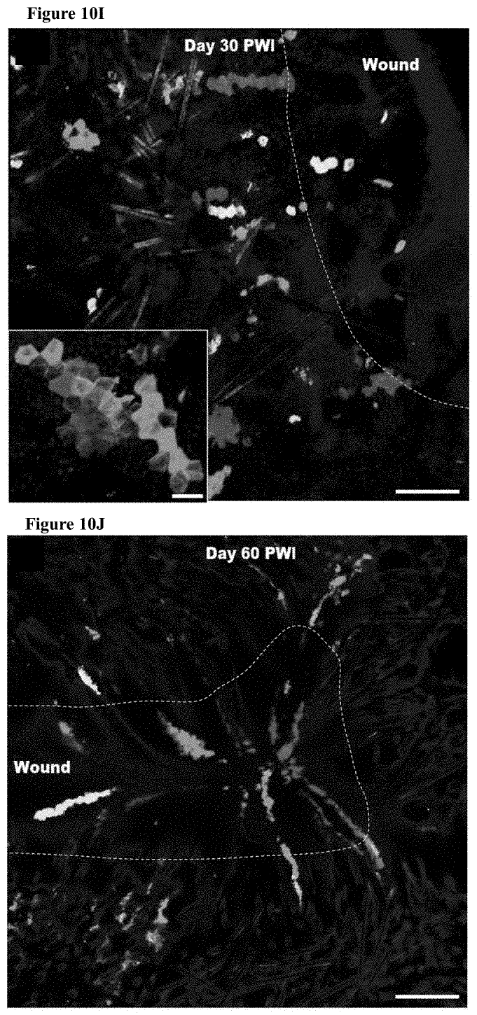

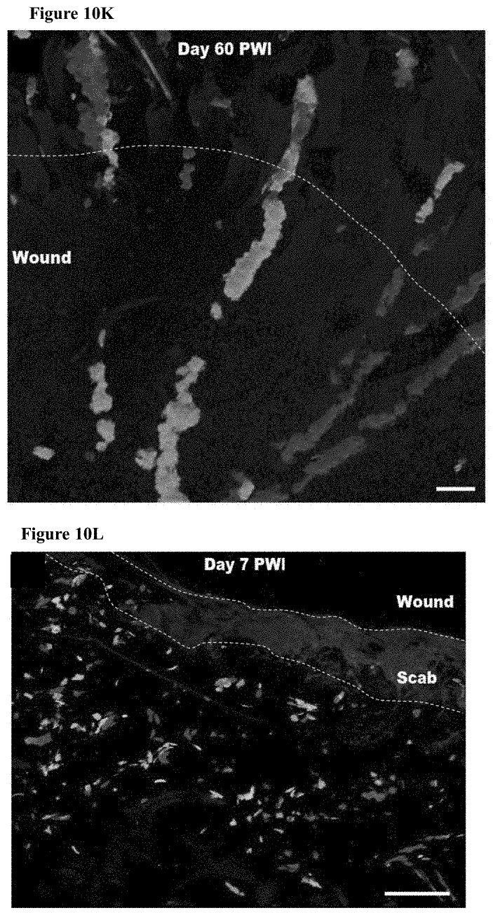

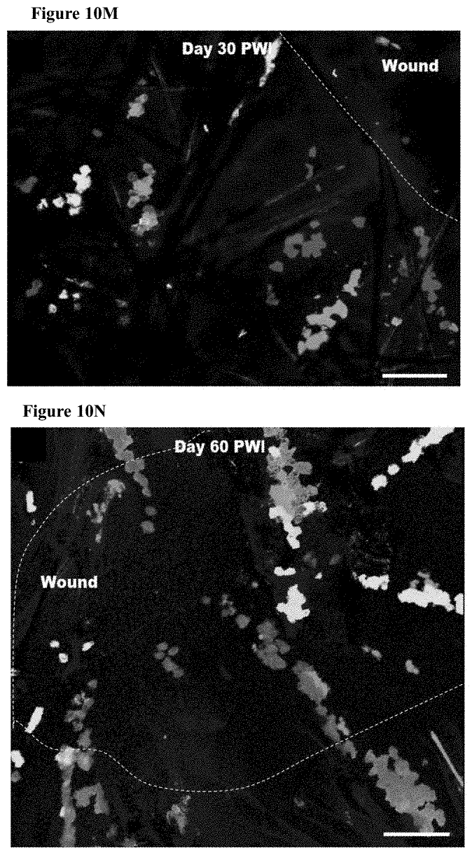

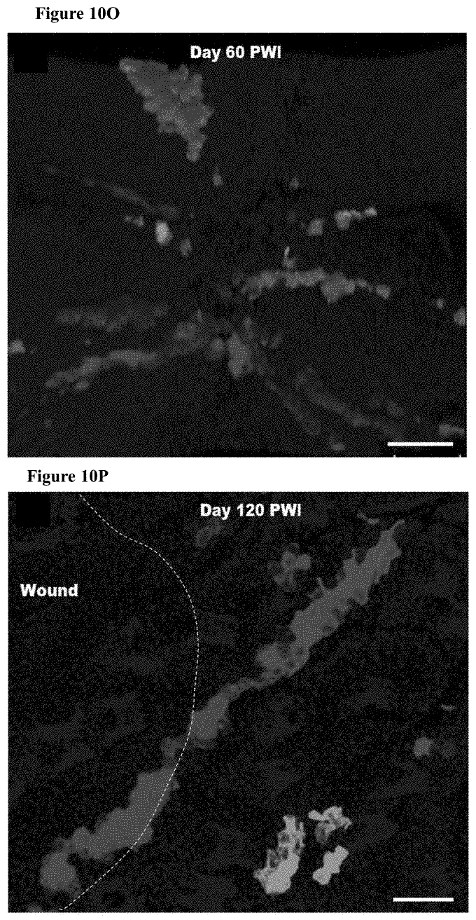

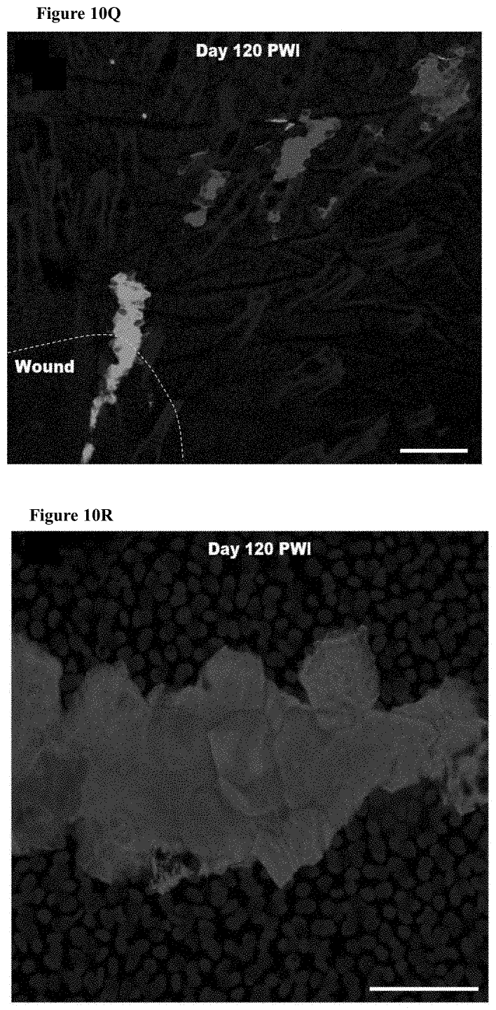

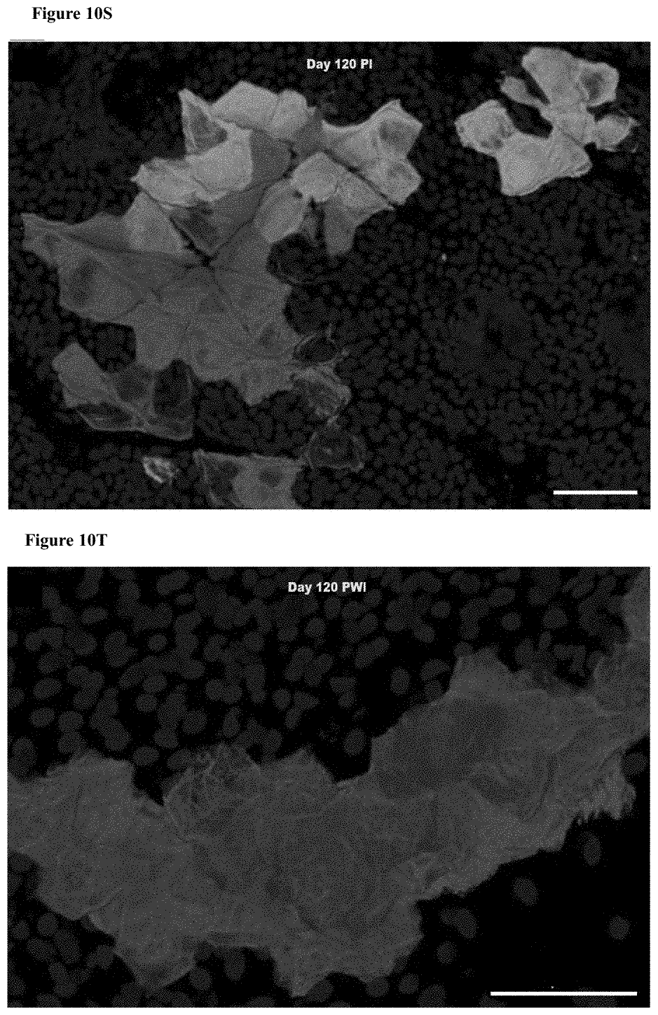

It was next examined if these Blimp1+ cell-derived organoids could be generated by isolating primary α6+; ScaI−; Blimp1−YFP+ cells and directly seeding them into Matrigel without a pre-culturing phase. These attempts did not give rise to organoid structures presumably due to low cell survival through loss of cell-cell contact. Thus, these cultures were supplemented with a ROCK inhibitor (Y27632), which prevents cell-cell interaction-dependent anoikis. Under these culture conditions freshly isolated α6+; ScaI−; Blimp1−YFP+ cells were able to give rise to organoids, although at a lower efficiency (FIG. 10-P).

### Example 2: Blimp1−YFP⁺ Cell-Derived Organoids Express Sebaceous Gland Markers

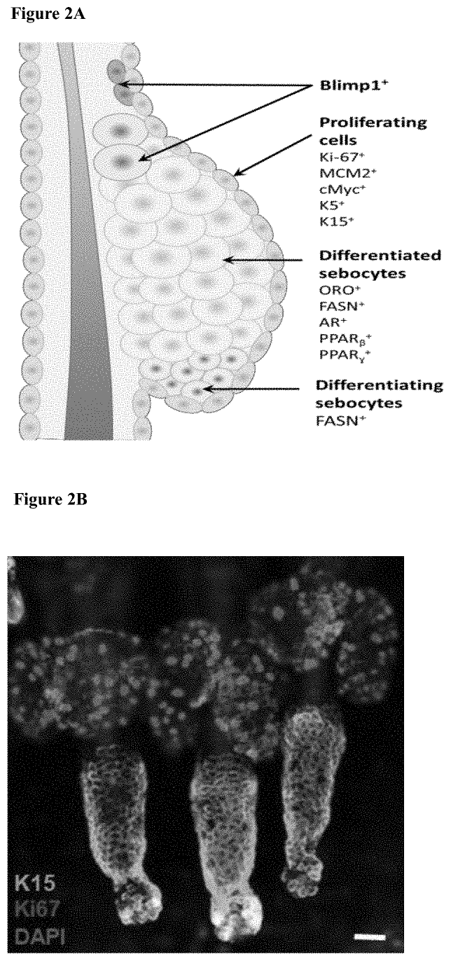

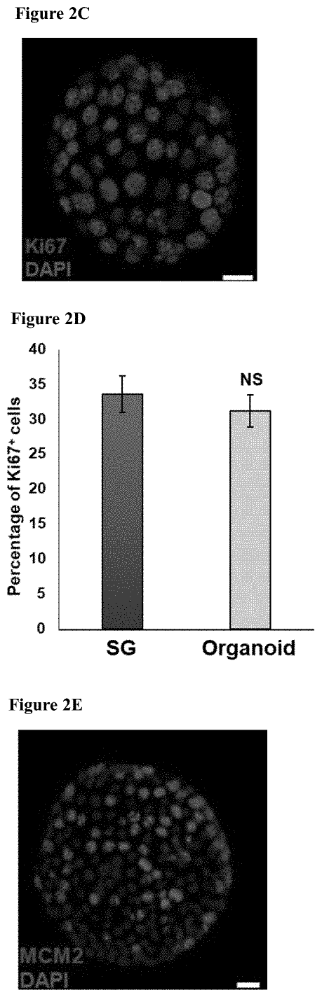

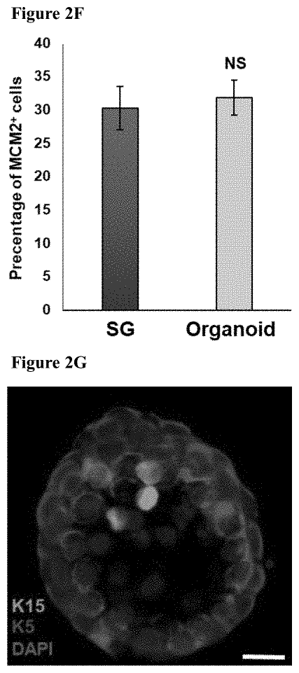

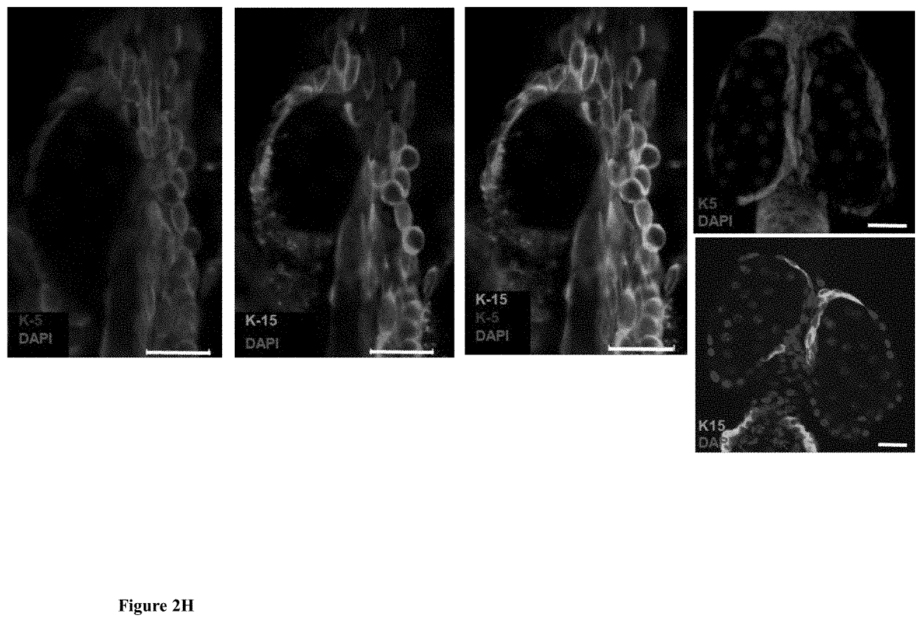

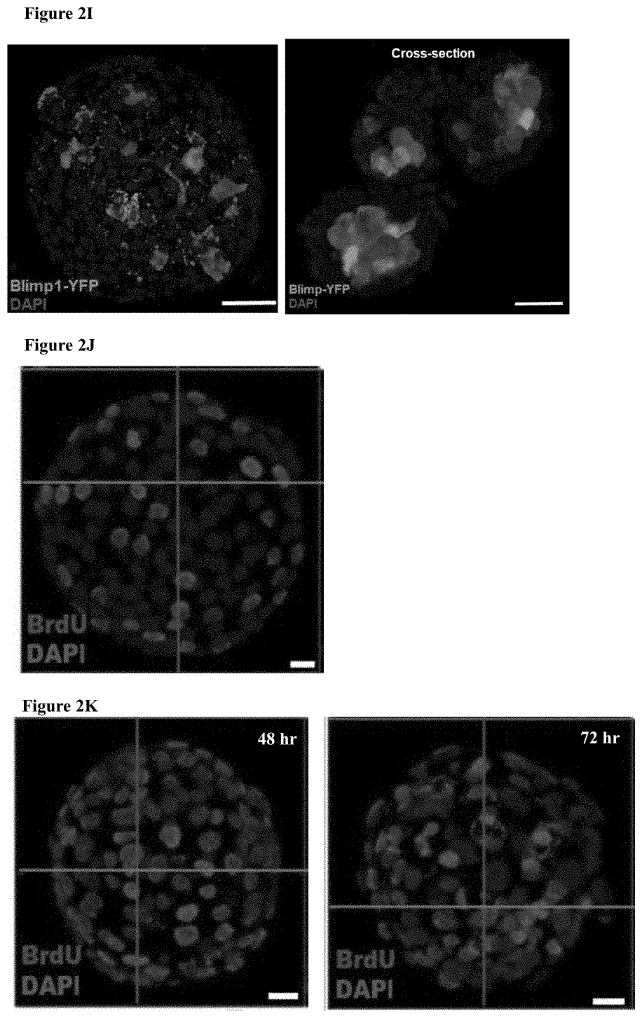

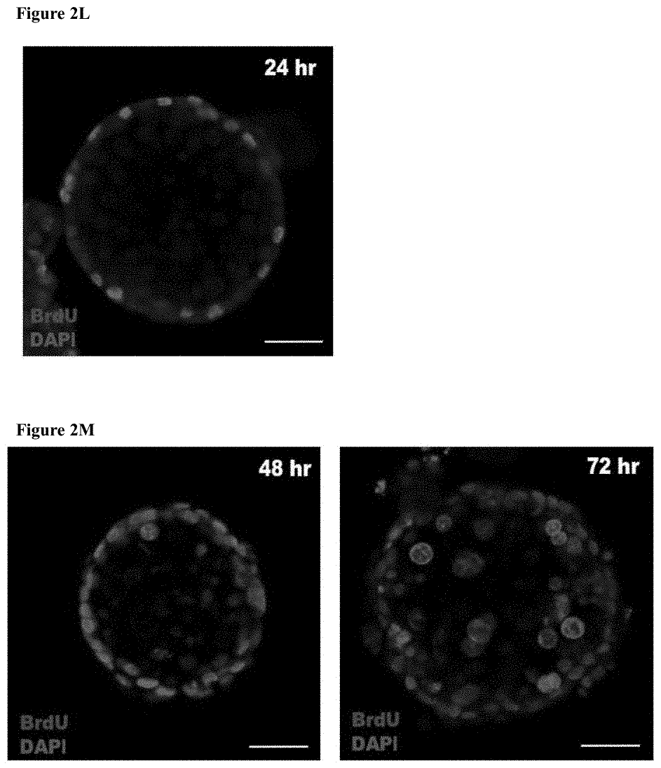

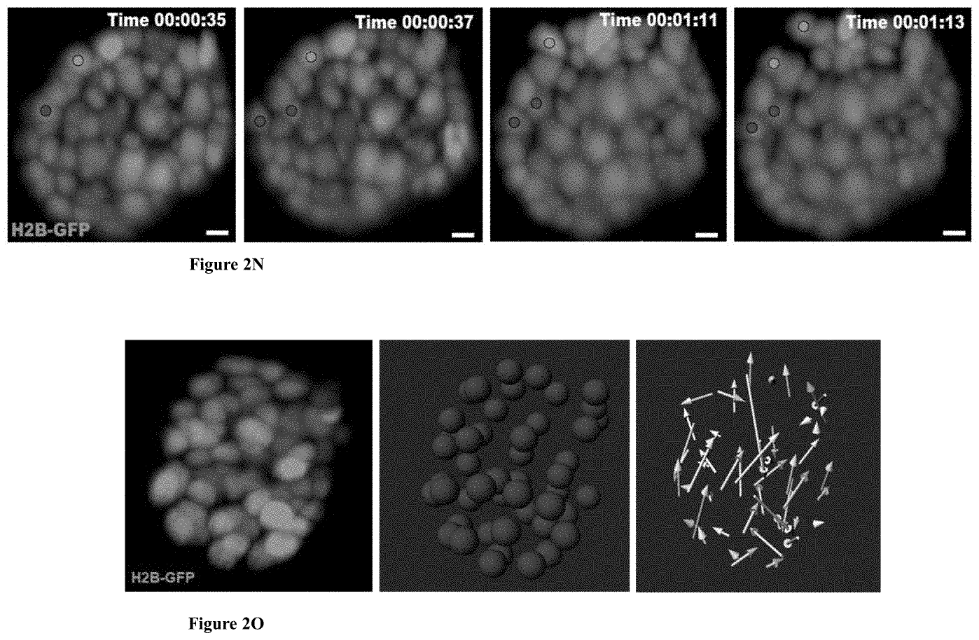

The basal layer of the SG is composed of proliferating cells situated along the SG proliferative zone (SGPZ), which differentiate and give rise to lipid-filled sebocytes in the inner compartment of the SG (FIG. 2A). In order to examine whether these organoid structures recapitulate normal SGs, first immunofluorescence (IF) with the specific proliferation marker, Ki67, known to mark cells of the SGPZ12 was performed. As expected, analysis of skin wholemounts showed that Ki67 is specifically expressed in the outer layers of the SG and could rarely be seen in quiescent bulge HFSCs (FIG. 2B). Quantitative analysis indicated that ˜34% of cells in the SGPZ were positive for Ki67. Similarly, Ki67 marked cells in the outer layer of Blimp1+-generated organoids at a similar frequency (FIG. 2C-D). These results were also verified using the MCM2 proliferative marker (FIG. 2E-F).

An important component of the cytoskeleton of epithelial cells is Keratin 5 (K-5), which is primarily expressed in basal keratinocytes of the epidermis. Similar to normal SGs in vivo, Blimp1+ cell-derived organoids expressed K-5 in the outer layer (FIG. 2G-H). Additionally, performing IF in both dorsal and tail wholemounts with K-15 antibody, it was found that K-15 labels specific cells at the base of the SG and junctional zone as well as individual cells in the outer layer of the organoids (FIG. 2G-H).

Next, the expression of Blimp1, which is expressed in both cells at the base of the SG as well as in mature sebocytes in vivo was examined (FIG. 1A). In the organoid cultures Blimp1−YFP+ cells were observed in both these settings. These findings indicate that the Blimp 1 promoter is active in organoids, supplying further evidence for the similarity to natural SGs (FIG. 2I).

Since proliferating cells could only be seen at the outer layer, whether they could give rise to cells in the inner compartment was investigated by monitoring movement kinetics in the organoids. Conducting pulse-chase 5-Bromo-2′-deoxyuridine (BrdU) experiments, it was found that 24 hours after a BrdU pulse only cells located on the organoid outer layer were positive for BrdU (FIGS. 2J and 2L). This is in accordance with the Ki67 and MCM2 staining (FIGS. 2C and 2E). In contrast, after 48 and 72 hours BrdU positive cells could clearly be detect in the inner non-proliferating mass, indicating that cells from the outer layer either migrated or proliferated asymmetrically and gave rise to differentiated post-mitotic cells (FIGS. 2K and 2M).

Next, in order to investigate the movement kinetics in real time, time lapse imaging was performed using light sheet microscopy. First, Blimp1−YFP+ cells were infected with retroviruses encoding H2B-GFP, which labels the nucleus. Then organoids were grown for 7 days and visualized for 24 hours. The results indicate that the majority of cellular divisions were along the peripheral ring (˜96%), however asymmetrical divisions could also clearly be detected (˜4%) (FIG. 2N-O). Thus, our data reveals that Blimp 1+ cell-derived organoids mimic both the expression profile, as well as homeostasis kinetics, as seen in SGs in vivo.

### Example 3: Blimp1−YFP⁺ Cell-Derived Organoids have a Lipidomics Signature Resembling SGs In Vivo

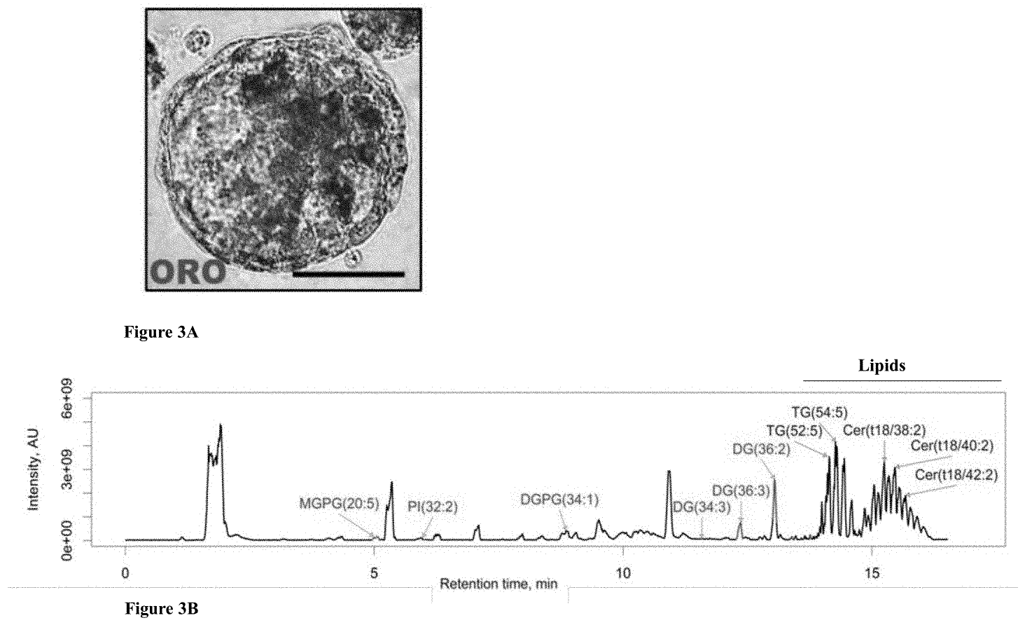

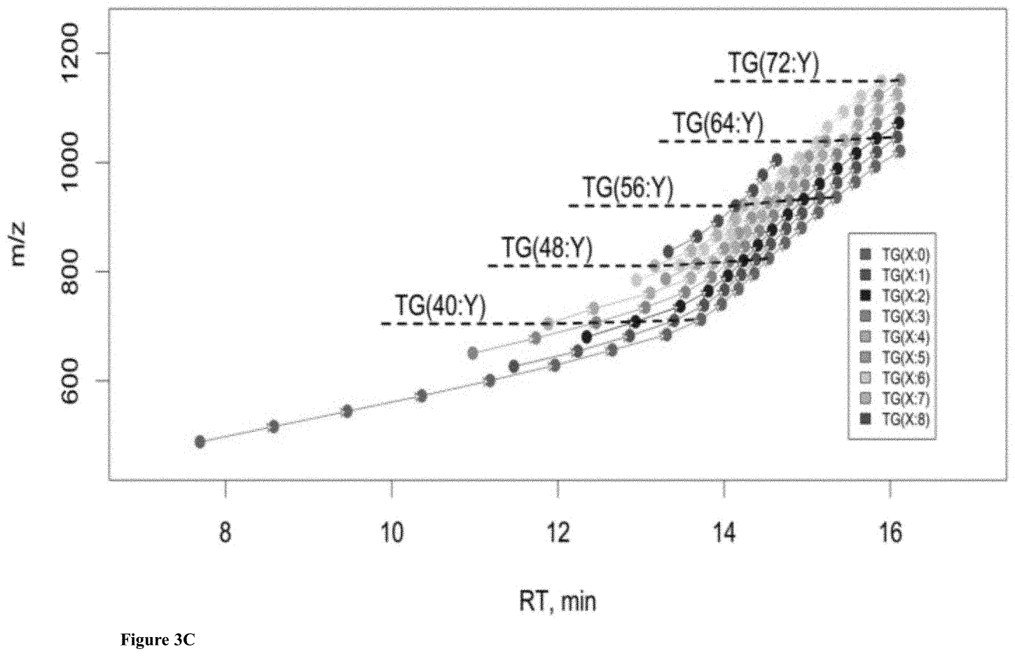

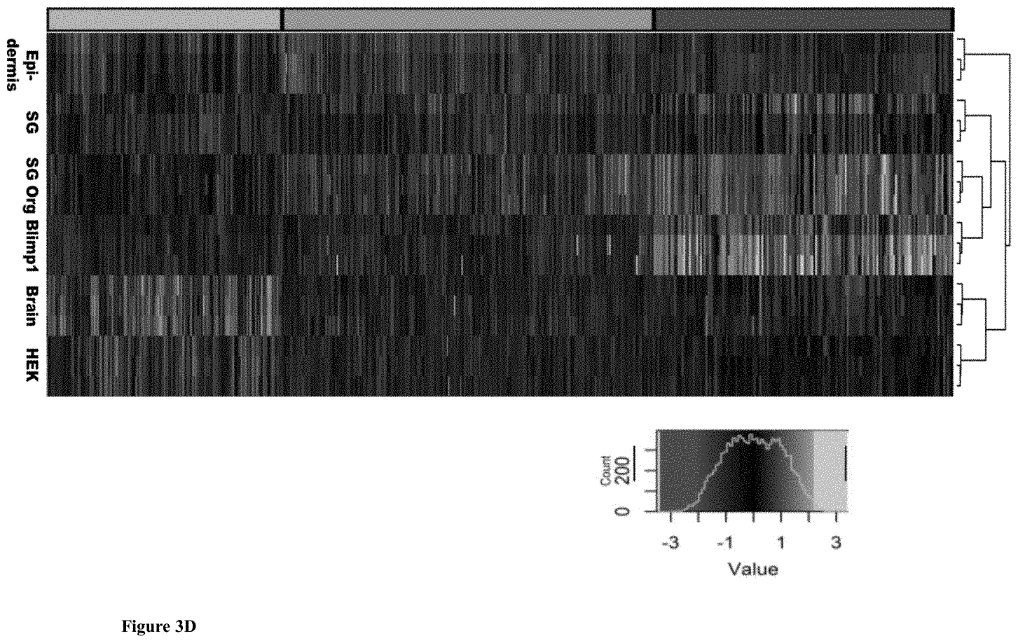

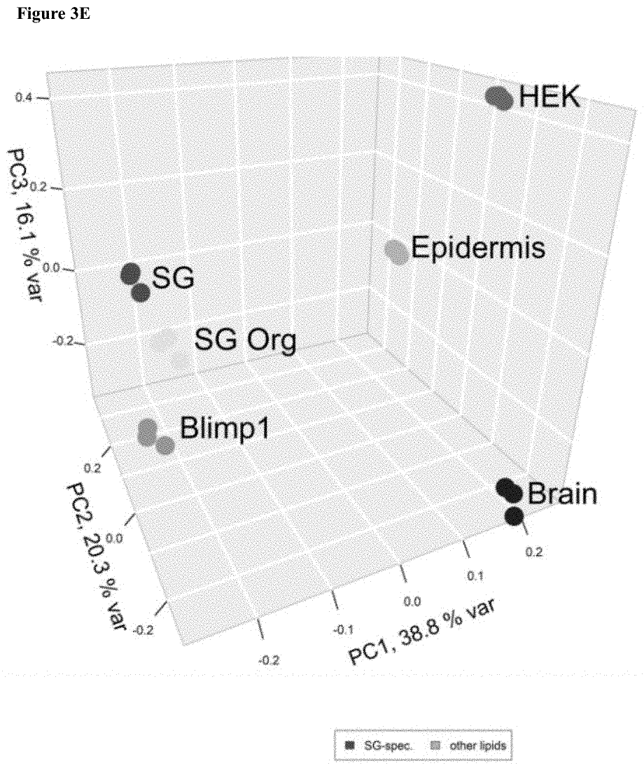

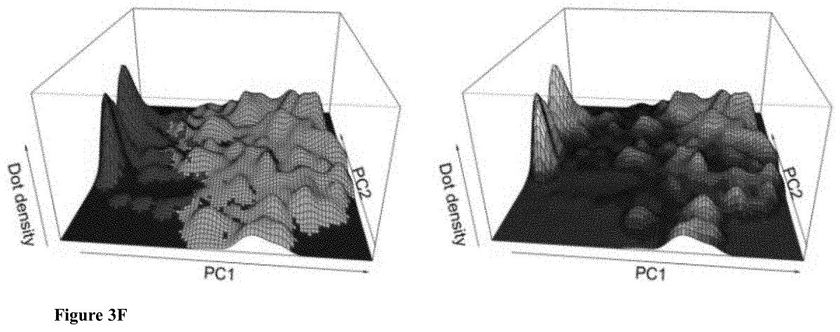

One distinct feature of a SG is the unique profile of lipids that it generates. Using the lipid dye, Oil red O (ORO), it was found that Blimp+ cell-generated organoids encompass lipid-producing cells in the inner mass (FIG. 3A). The observation that organoid inner cells do not proliferate, and but do express lipids is suggestive of a differentiation program similar to the SG. Therefore, in order to examine whether these organoids generate lipids which resemble a normal SG in vivo, lipidomic analysis was performed utilizing HPLC coupled with mass-spectrometry (LC/MS). A comparison of lipid composition between: 1) whole epidermis (Epi), 2) resected SGs, 3) Blimp1⁺ cells grown in 2D and 4) Blimp1+ cell-derived organoids was performed. Additionally, there was included highly unrelated samples: HEK293T cells and mouse brain (designated HEK/BR). Lipids were extracted in three biological replicates by Folch procedure and were subjected to C18 chromatographic separation coupled with electrospray ionization high-resolution mass spectrometry analysis (FIG. 3B). 6470 peaks were identified, representing single-charged molecular ions in both polarity modes ([M+H]+, [M+NH4]+, [M−H]−, [M+HCO2]). Large classes of lipids were observed as series of peaks separated by the mass (m/z) of C₂H₄ segment with a shift to longer retention times (RT) for longer acyl chains and higher saturation levels (FIG. 3C). Normalized intensity profiles of identified lipid compounds were used to quantify content similarity between samples. Similarity in total lipidome was addressed by hierarchical clustering (FIG. 3D) and principle component analysis (PCA) (FIG. 3E). Hierarchical clustering grouped together resected SGs, 2D-grown Blimp1⁺ cells, and Blimp1+ cell-derived organoid samples, while the HEK293T cells/brain and epidermis samples represented two additional distinct groups (FIG. 3D-E). A significant portion of detected lipids could then be grouped based on their elevated relative abundance within a combination of sample types: SGs, Blimp1+ cell-derived organoids and 2D-grown Blimp1⁺ cells (cluster I), epidermis, SGs and Blimp1+ cell-derived organoids (cluster II) and epidermis, HEK293T cells and brain (cluster III; FIG. 3D). Of note, cluster III was clearly distinguishable from the SGs/organoids/Blimp1⁺ cells (cluster I) profile (FIG. 3D). Importantly, in this analysis the production of a specific spectrum of lipids in both normal SGs and in Blimp1⁺ cell-derived organoids was clearly detected (FIG. 3D).

The PCA analysis of mass spectrometry data further supported lipidome characterization, by placing the first principle component (PC1) as direction differentiating SG, SG-Org, and 2D-grown Blimp1 cells from the epidermis and HEK/BR group of samples (FIG. 3E). The second principle component (PC2) differentiated samples of brain/HEK versus epidermis and SGs/organoids/Blimp1+ samples. The first two principle components together explain more than half of the variance in the data. On the PCA score plot, SGs/organoids/Blimp1+-specific compounds are distinctly separated from the groups of epidermis and HEK/brain-specific compounds (FIG. 3E). The density distribution of PCA scores is represented by a landscape with deep valleys separating the SGs/organoids/Blimp1+-specific peaks versus all others, indicating the distinction and the strong cross correlation of the SG-like lipids (FIG. 3F). Taken together the lipidomic analysis indicates that SG organoids have the capacity to generate unique lipids, displaying a signature resembling that of SGs in vivo.

### Example 4: c-Myc Regulates Proliferation and Differentiation in the Sebaceous Gland Organoid

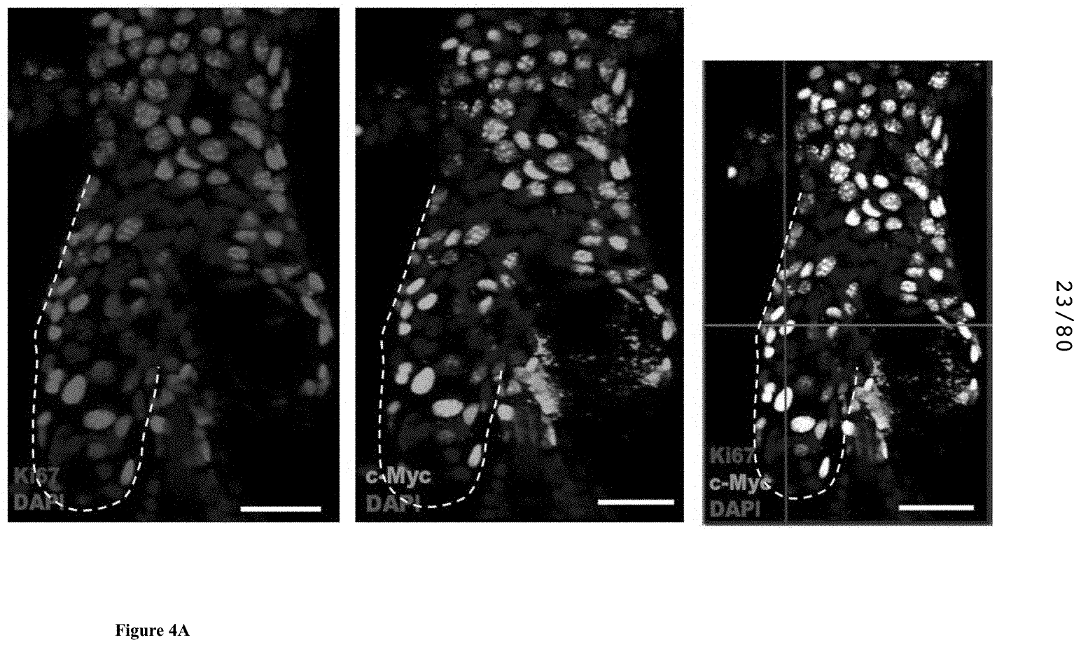

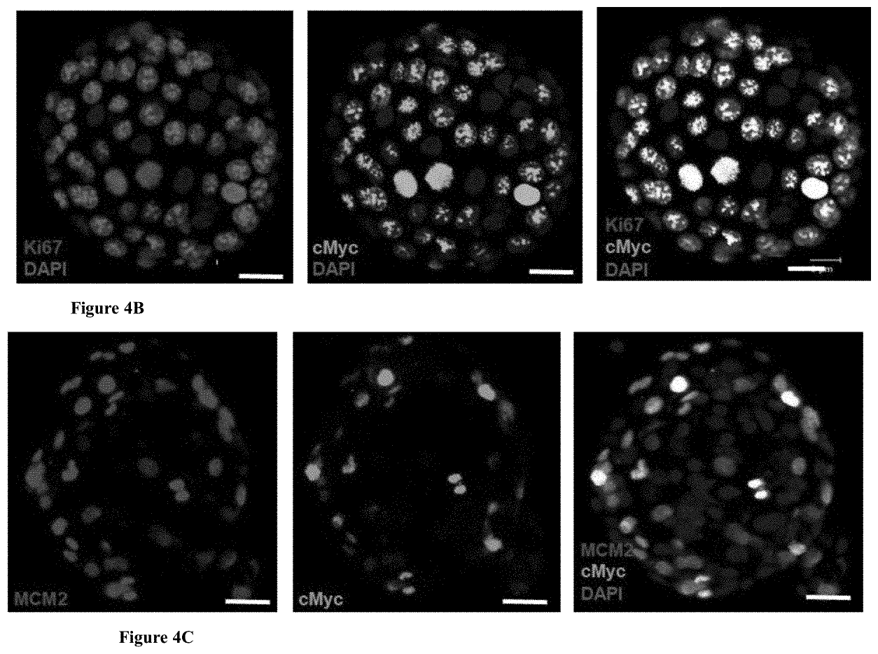

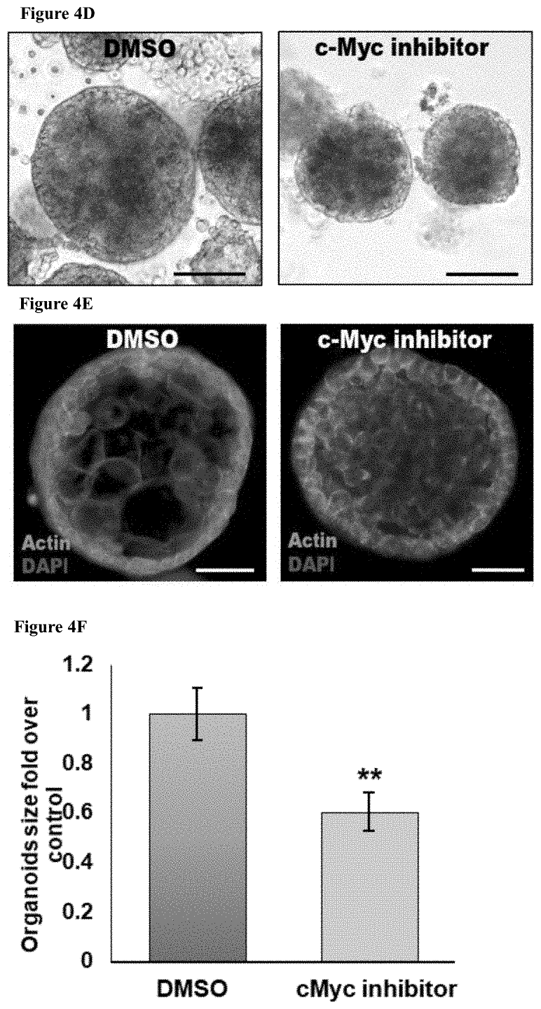

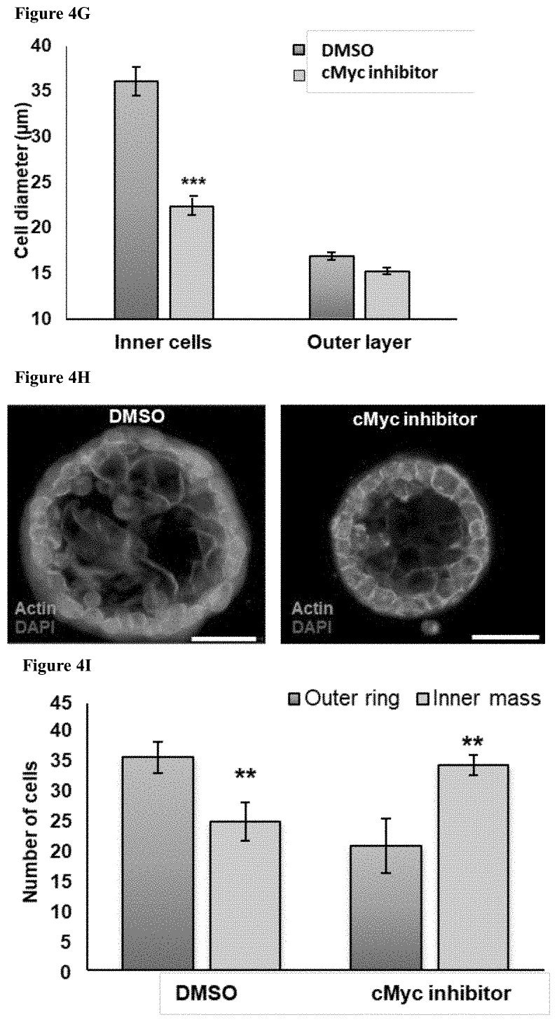

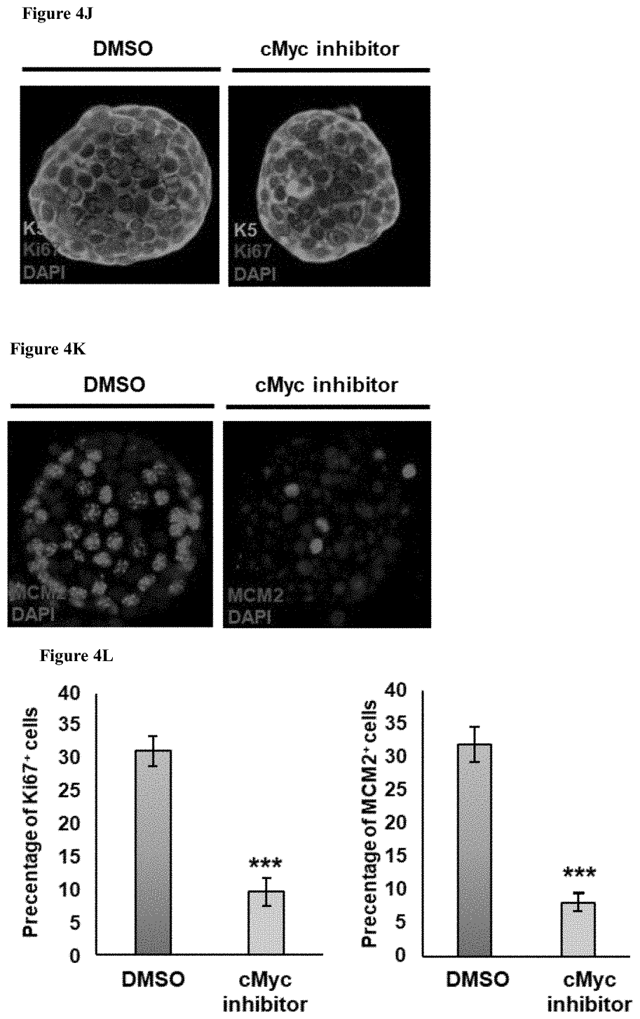

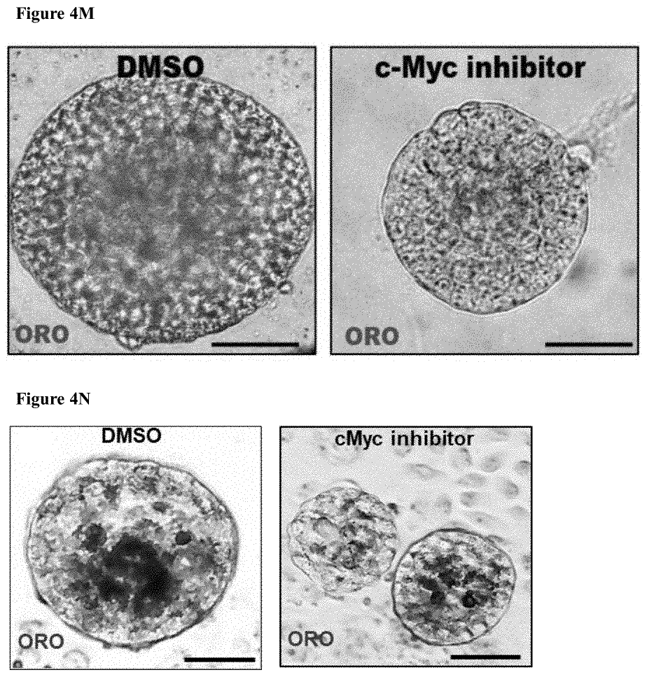

Given that Blimp1+ cell-derived organoids displayed morphological and functional similarity to normal SGs, it was next examined if this novel platform could be utilized to investigate signaling cascades that regulate SG homeostasis. One pivotal factor that governs SG expansion is c-Myc. In vivo, c-Myc labels cells in the SGPZ and regulates both sebocyte proliferation and differentiation (FIG. 4A). As in normal SGs, the expression of c-Myc mostly co-localized with Ki67 and MC2M in the SGPZ and was not seen in the inner compartment of the SG organoids (FIG. 4B-C).

Next, the effect of inhibiting c-Myc activity by administering the specific c-Myc inhibitor, 10058-F4 was monitored. This chemical inhibitor does not prevent the translocation of c-Myc to the nucleus but specifically inhibits the c-Myc-Max interaction and prevents transcription of c-Myc target genes. Six-day old SG organoids were supplemented with the c-Myc inhibitor for 4 days. As expected, c-Myc inhibition did not affect the localization of c-Myc, however it resulted in a ˜40% decrease in organoid size (FIG. 4D-F). Since the effect on organoid size could result from either altered cell size or number both scenarios were examined. Quantifying the diameter of individual cells in the outer proliferating layer of the organoid revealed a modest size decrease (FIG. 4G). However, inhibition of c-Myc had a strong effect on the dimeter of cells in the inner compartment, resulting in a ˜40% decline in cell diameter (FIG. 4E, 4G). In complement, it was found that the number of cells in the outer ring of the c-Myc-inhibited organoids was decreased, while the inner zone encompassed cells of smaller size (FIG. 4E, 4G-I). These results are in accordance with previously published findings that overexpressing c-Myc results in enhanced SG expansion, while c-Myc deletion leads to decreased SG size.

Whether the effect of c-Myc inhibition originates from an effect on cell proliferation and/or differentiation was examined next. Employing IF staining with two proliferative markers, Ki67 and MCM2, indicated a significant decline in Ki67+ (69%) and MCM2+ (72%) proliferative cells (FIG. 4J-L). This suggests that proliferation and expansion of the SG organoids is dependent upon c-Myc activity.

Importantly, the data revealed that upon c-Myc inhibition, cells in the inner compartment were significantly smaller than their control counterparts, suggesting a possible effect on cell differentiation. This is in line with the reported role of c-Myc in regulating differentiation in the SG in vivo. Therefore, as a first step lipid staining with the ORO dye was performed, which demonstrated a significant decrease in lipid levels (FIG. 4M-N). Next, using Real time (RT)-PCR analysis, the levels of transcripts that are expressed in either SG differentiating or differentiated cells, which play a key role in this process were examined. Inhibition of c-Myc decreased the levels of all examined factors including Androgen Receptor (AR) and fatty acid synthase (FASN) (FIG. 4O). Furthermore, the central transcription factors peroxisome proliferator-activated receptor −β and −γ (PPAR-β and PPAR-γ,) which govern numerous genes involved in lipid synthesis and regulate sebocyte differentiation and maturation, were also significantly attenuated (FIG. 4O). These data show that, similar to normal SGs, c-Myc plays an important role in regulating both proliferation and differentiation of the SG organoid.

### Example 5: Modeling the Interfollicular Epidermis, Thy1.2 Expression Pattern

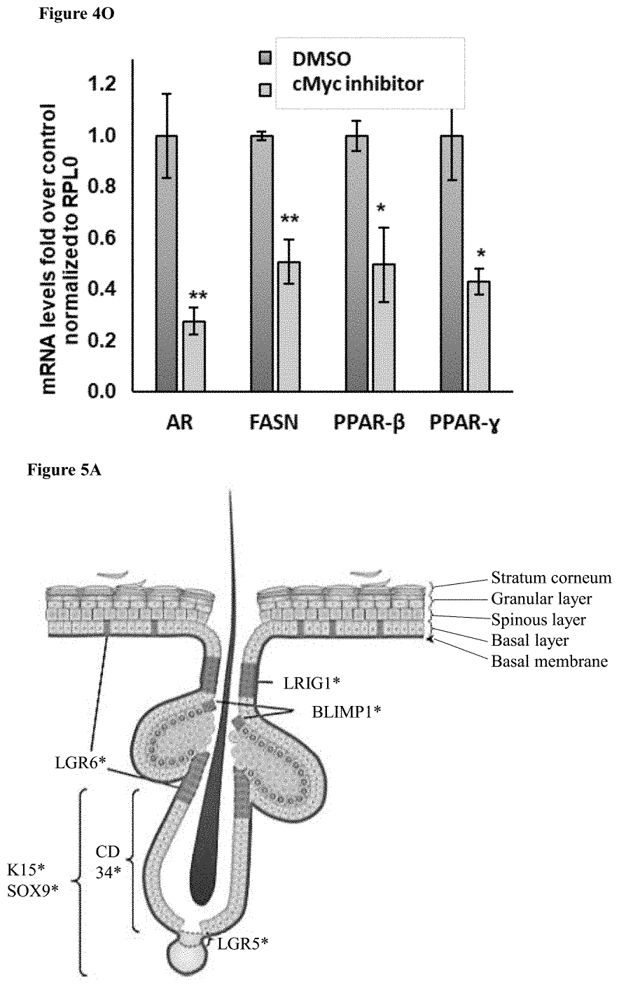

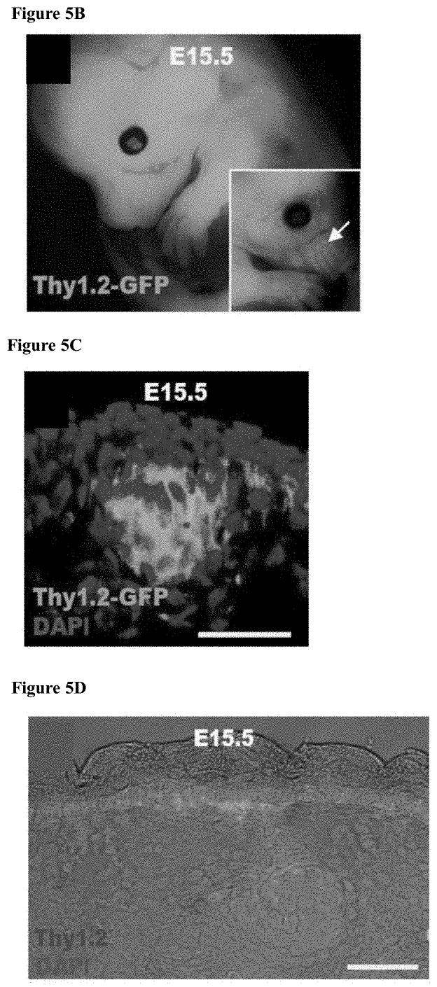

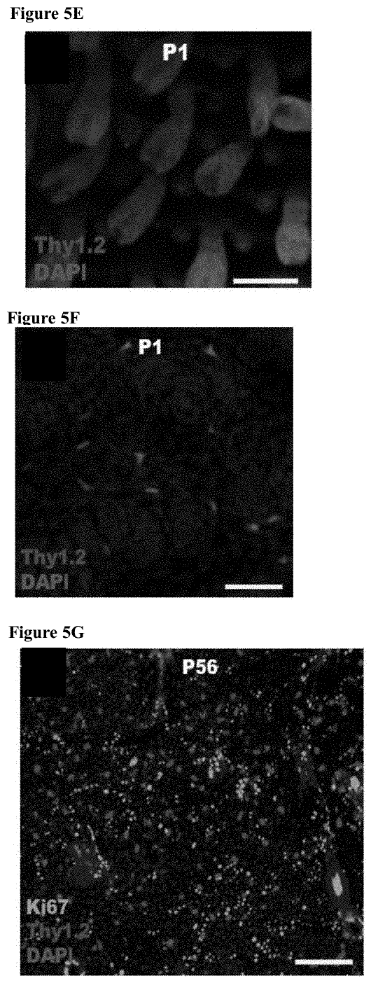

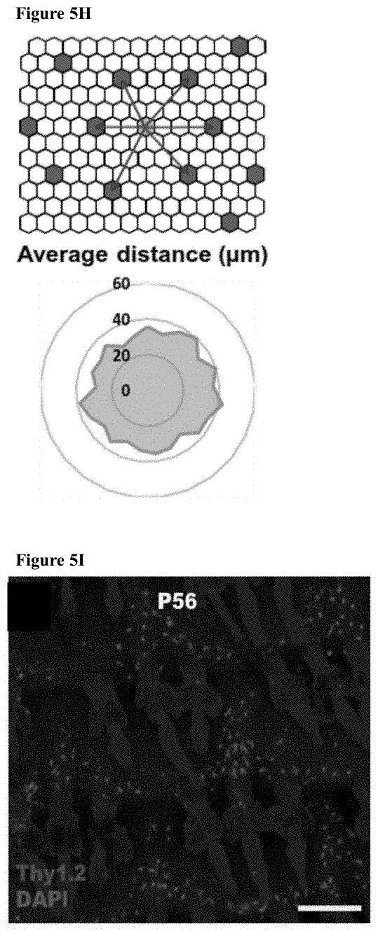

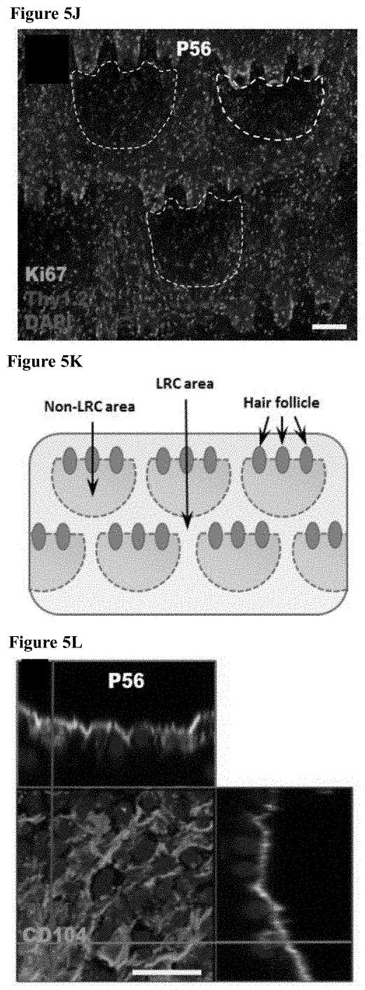

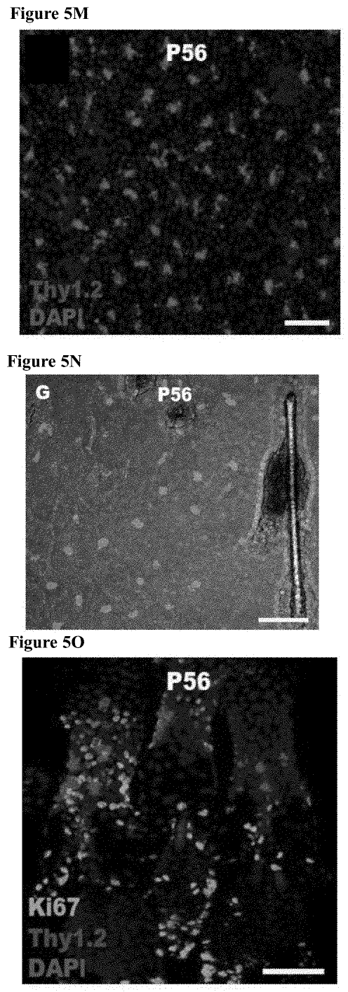

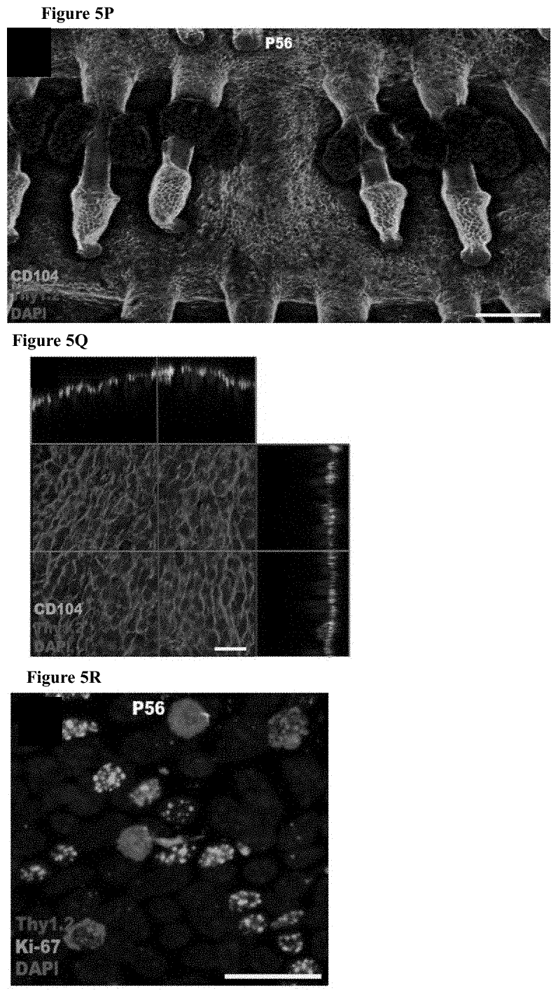

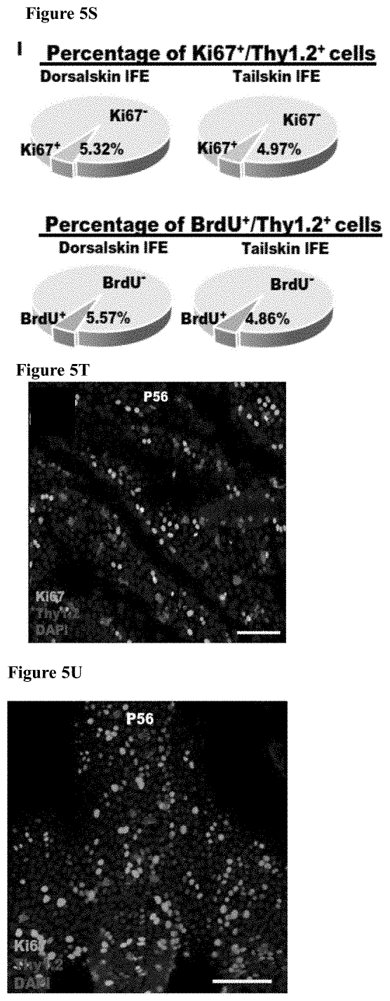

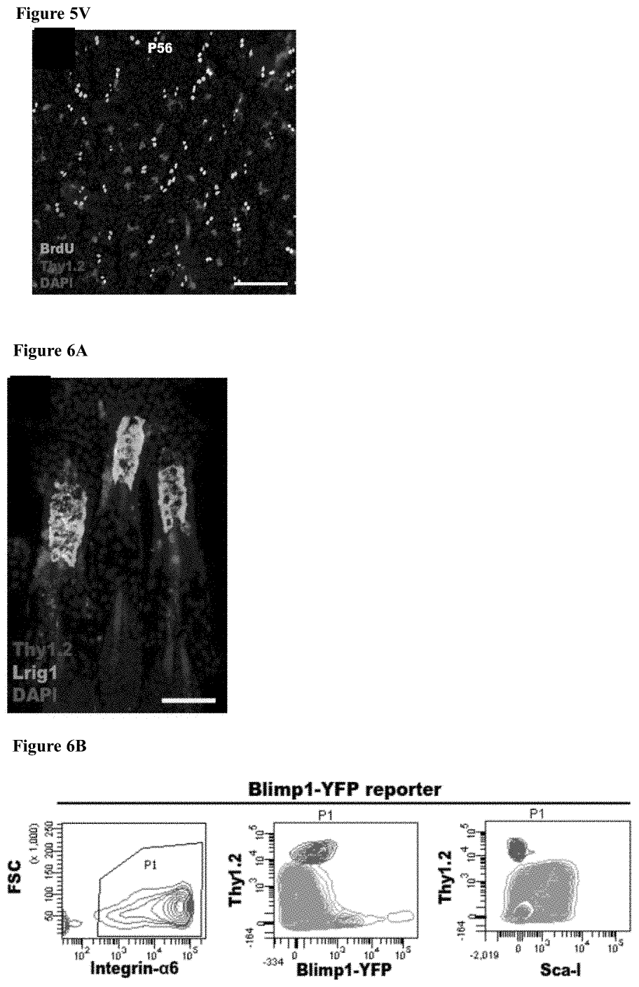

Several progressively differentiated layers of epithelial cells constitute the interfollicular epidermis (IFE). The inner basal layer consists of proliferating cells that undergo terminal differentiation, giving rise to suprabasal cells that gradually shed from the skin surface (FIG. 5A). It has been proposed that the IFE is organized in distinct columns known as epidermal proliferating units (EPUs). According to this model each unit is autonomously maintained by a slow-cycling stem cell (SC) that lies in its center and gives rise to a pool of transit-amplifying progenitors with defined proliferative potential.

In a pilot RNA-Seq screen of skin epithelial populations, the Thy1.2 gene was found to be transcribed in basal keratinocytes. Intriguingly, human Thy1.2 is also expressed in a subset of epidermal keratinocytes presumably with stem/progenitor features. In mice, Thy1.2 is best known as a marker for neuronal cells and has been utilized for the visualization of neurons in the central nervous system and the creation of the Brainbow mouse. In order to evaluate a potential involvement of Thy1.2 in the epidermis, Brainbow 2.1 mice that express GFP in Thy1.2+ cells prior to Cre recombination were used. The artificial promoter of Thy1.2 was designed in such a way that only cells highly expressing Thy1.2, which supposedly occurs only in neurons, could drive the reporter. As expected, Thy1.2-GFP was highly expressed in the brain of Brainbow 2.1 mouse (FIG. 5B). Interestingly, as early as embryonic day 15.5 (E15.5) Thy1.2 expression was also observed in placodes, throughout the IFE and in the recruited fibroblasts (FIG. 5B-D). Moreover, Thy1.2 expression persisted during hair peg development (FIG. 5E) and was detected in distinct cells in the IFE at postnatal day 1 (P1; FIG. 5F). Immunofluorescent staining of 8-week old (telogenic) mouse dorsal skin for Thy1.2 revealed that Thy1.2+ cells are evenly distributed throughout the IFE (FIG. 5G-H, M). The distance between Thy1.2+ cells was found to be relatively constant, spanning between 26±10 μm (FIG. 5H). Additionally, Thy1.2+ cells were also detected in the infundibulum (IFD) and in the HF junctional zone, although no staining was seen in the SG nor other compartments of the HF including the bulge, isthmus and hair germ (FIG. 5G, N-O). Staining for Thy1.2 was also performed in the tail skin. Here, Thy1.2 was similarly and predominantly expressed in the IFE. However, the distribution was not homogenous and labelled cells were detected almost exclusively in a distinct territory recently reported to house label retaining cells (LRCs) (FIG. 5I-K). In order to verify these findings, the non-label retaining zone, which is obscured by HFs, was examined and no Thy1.2+ cells were found in this compartment (FIG. 5J-K). Importantly, in the IFE of both dorsal and tail skin Thy1.2+ cells were localized to the basal layer and were not detected in the differentiated suprabasal layers (FIG. 5L, 5P-Q). Co-staining with the proliferation marker, Ki67, was performed which revealed that in contrast to surrounding IFE basal cells, Thy1.2+ cells are relatively quiescent (FIG. 5R-V). In both dorsal and tail skin ˜5% of Thy1.2+ cells were positive for Ki67 (FIG. 5S). Similar results were also obtained with BrdU after a 24 hour chase period (FIG. 5S, 5V).

### Example 6: Thy1.2 Marks a Distinct IFE SC Population

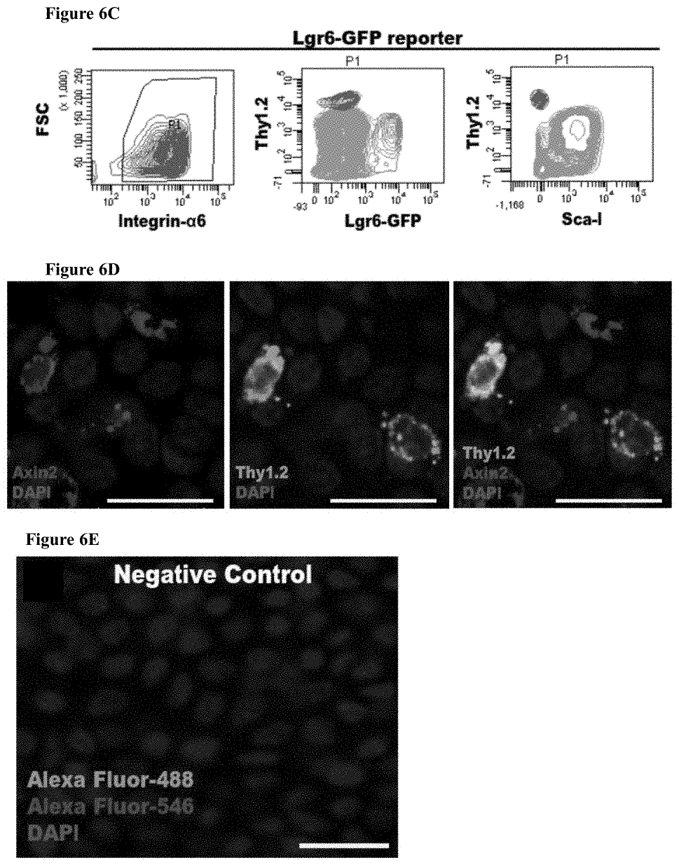

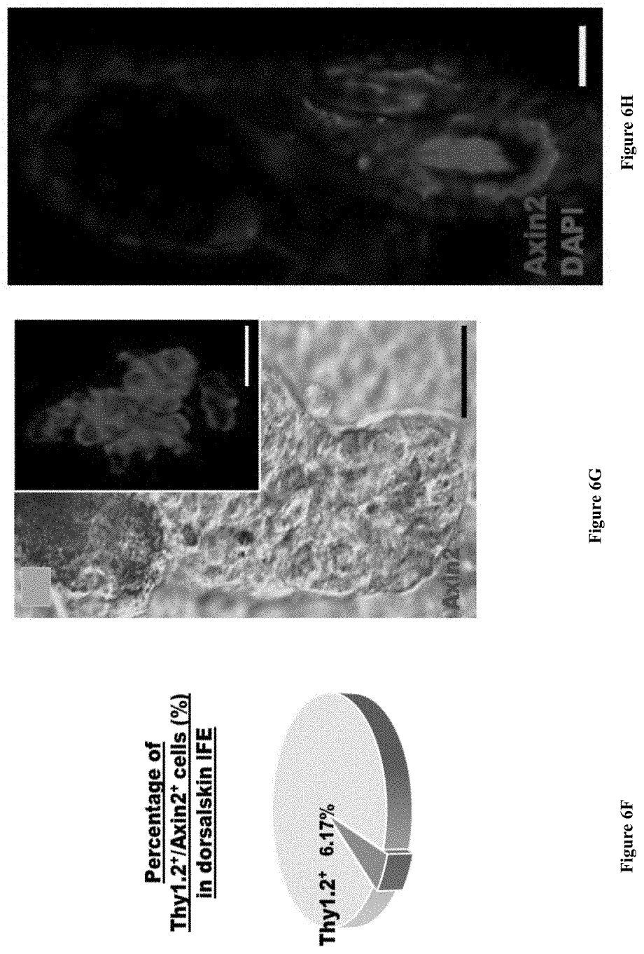

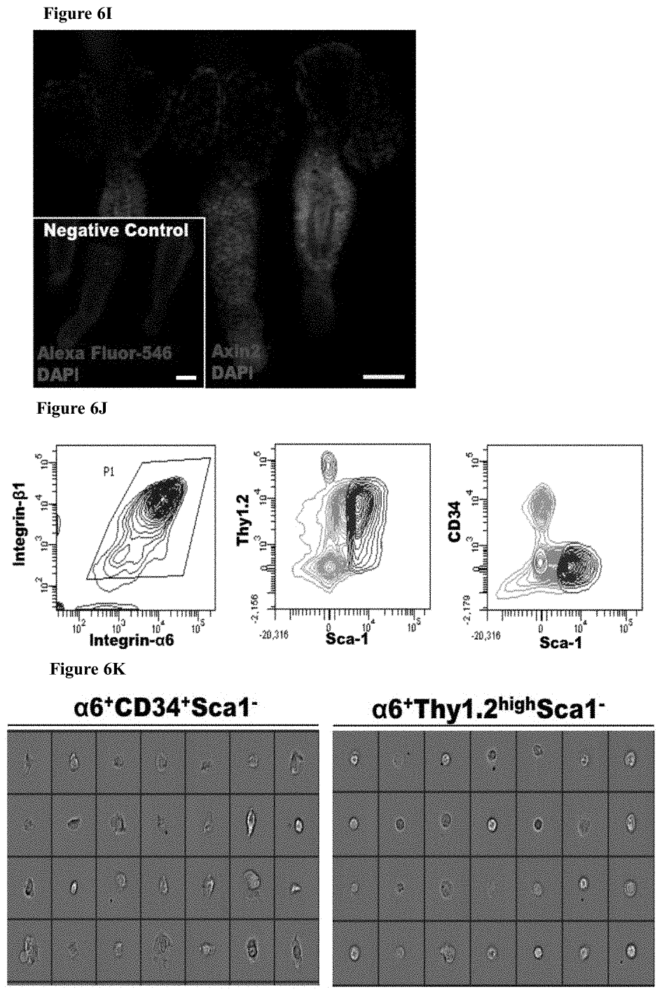

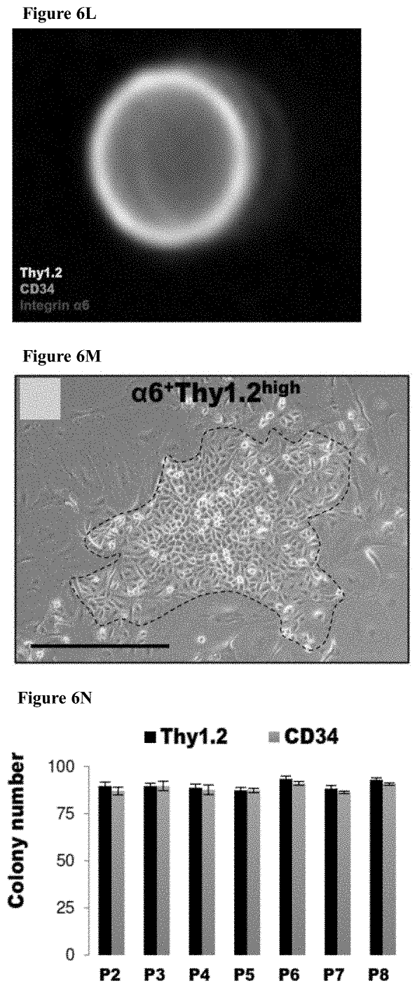

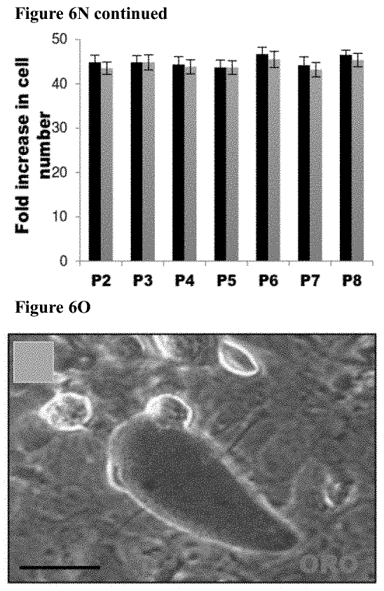

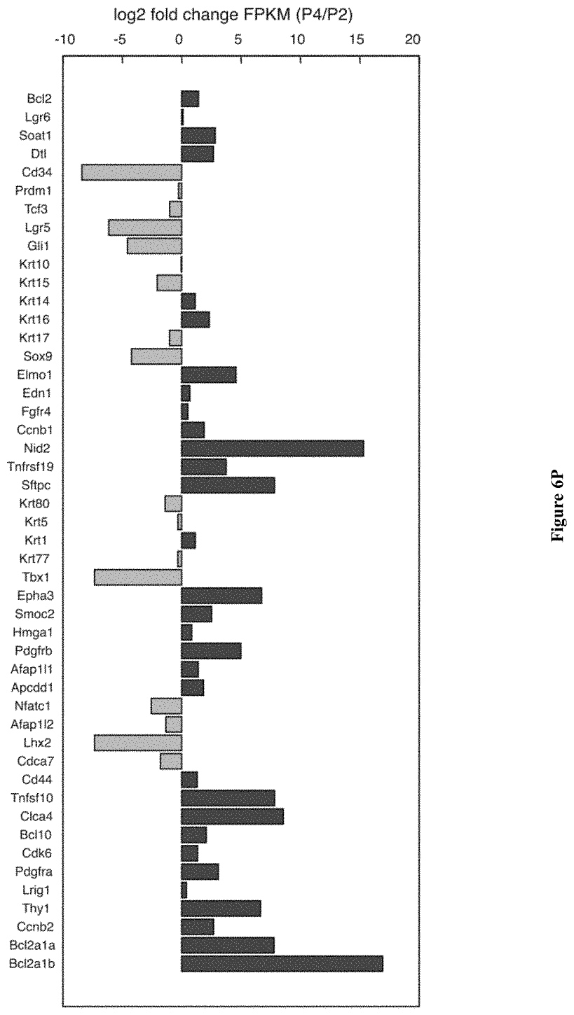

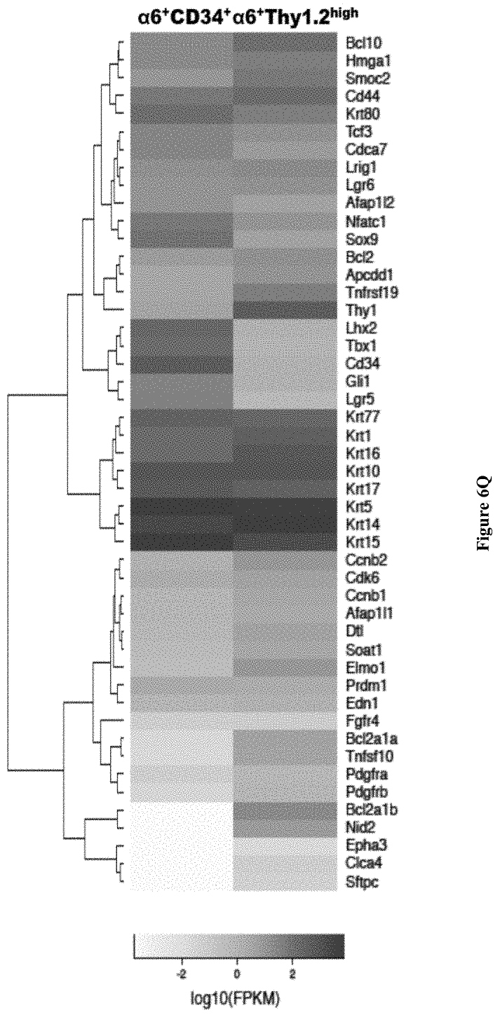

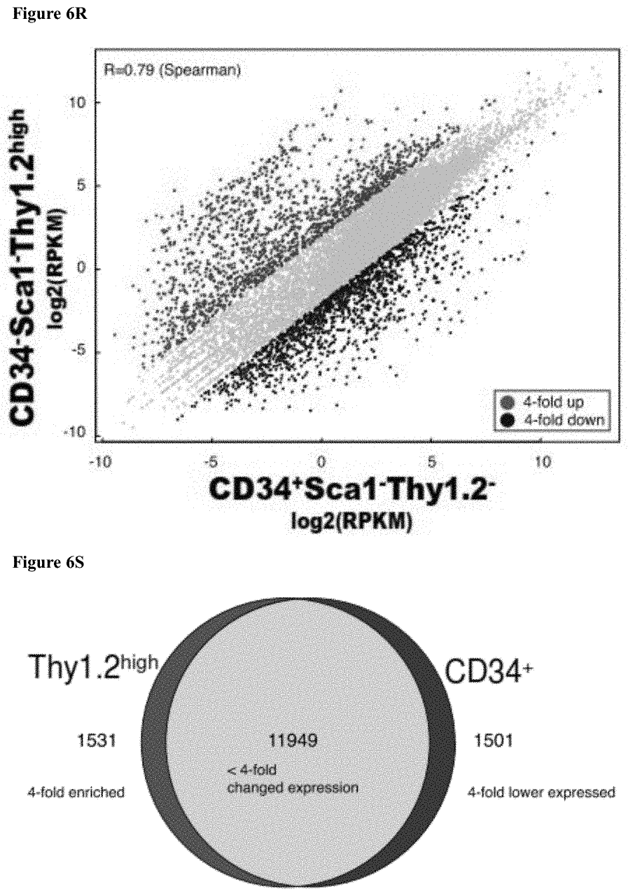

Since Thy1.2 marks a distinct epidermal population of quiescent cells, whether this population is distinct from other known stem and progenitor populations was examined. Given that Thy1.2 marks cells in the IFD, junctional zone and IFE Lgr6, Blimp1 and Lrig1 populations that have been reported to reside in close proximity were examined first. For this purpose, co-labeling with the Lrig1 antibody was performed and it was found that only an extremely minor fraction of Lrig1+ cells were positive for Thy1.2 (FIG. 6A, ˜%0.25). As expected, these Thy1.2+Lrig1+ cells were only seen in the IFD and junctional zone but not in the IFE.

Next, fluorescence activated cell sorting (FACS) employing Blimp1 and Lgr6 reporter mice (B6.Cg-Tg(Prdm1-EYFP)1Mnza and B6. 129P2-Lgr6tm2. 1 (cre/ERT2)Cle/J, respectively) was used. For analysis antibodies for integrin α6, Thy1.2 and ScaI were utilized, which revealed a population of α6+ cells that express high levels of Thy1.2 (designated Thy1.2high). Importantly, these Thy1.2high cells were completely distinct from both Blimp1⁺ and Lgr6+ cells and no overlap was seen (FIG. 6B-C). These findings indicate that although Thy1high, Blimp 1+ and Lgr6+ populations are in very close proximity, they represent completely separate and distinct cell types.

Recently, Axin2 has been reported to mark a discrete SC population in the IFE and the HF bulge. Performing co-immunofluorescence in both dorsal and tail skin, a small fraction (˜6%) of Thy1.2+ cells in the IFE, but not in the HF bulge, were found to be positive for Axin2 (FIG. 6D-I).

Next, the Thy1.2high population was isolated and characterized by FACS using antibodies for integrin α6, (31, CD34, Sca-I and Thy1.2 (FIG. 6J). Approximately 0.3% of the α6+β1+ cells were highly positive for Thy1.2. Consistent with previous findings, Thy1.2 marked a population distinct from CD34 and no overlap was detected between the two markers (FIG. 6J). Interestingly, IFE and IFD keratinocytes also expressed Thy1.2, although at a lower level (designated Thy1.2low). Collectively, these results suggest that high expression of Thy1.2 marks a distinct epidermal cell population.

In order to examine the cellular distribution of Thy1.2 and the morphology of α6+Thy1.2high cells flow cytometry coupled with high-resolution microscopy was performed. It was found that these cells were highly uniform by morphology and that Thy1.2 protein was distributed throughout the plasma membrane. Furthermore, the circularity score, which examines the variability of the cell radius, was significantly higher than the score for both CD34+ HFSCs and Scal+ keratinocytes, suggesting limited morphological heterogeneity within the Thy1.2high population (FIG. 6K-L).

To further characterize the Thy1.2high population, α6+β1+CD34+Sca1−Thy− (bulge HFSCs), α6+β1+CD34−Sca1−Thy1.2high and α6+β1+CD34−Sca1+Thy1.2low (differentiated IFE/IFD cells) cells isolated from telogen-phase dorsal back skins were cocultured. After 2-3 weeks, their colony forming efficiency (CFE) (≥4 cells/colony) and cell numbers were evaluated. It was found that the CFE was approximately 1% and that the size of the Thy1.2high and CD34+colonies were relatively similar (FIG. 6M-N). Furthermore, serial dilution and long-term passaging indicated that, in contrast to IFE/IFD α6+β1+Sca1+Thy1.2low keratinocytes, which dwindled away after ˜4 passages, α6+β1+CD34+Sca1−Thy− and α6+β1+CD34−Sca1−Thy1.2high populations could be expanded over time, indicative of their SC features (FIG. 6N). Specifically, these cultures could easily be expanded for more than 40 passages. Interestingly, as opposed to α6+β1+CD34+Sca1−Thy− cells, some cells within the α6+β1+CD34−Sca1−Thy1.2high colonies displayed morphology reminiscent of sebocytes (FIG. 6N). Therefore, it was next examined whether α6+β1+CD34−Sca1−Thy1.2high colonies could generate sebocytes in vitro. Using oil red O (ORO) staining it was found that only α6+β1+CD34−Sca1−Thy1.2high cells, but not α6+β1+CD34+Sca1− cells, could yield ORO+ cells (FIG. 6O). The percentage of these ORO+ cells was approximately 0.1% of the total cell count (FIG. 6O). These findings show that Thy1.2high cells can self-renew and give rise to differentiated sebocytes in culture.

Next, the gene expression profiles of α6+CD34+Sca1−Thy−HFSCs and CD34−Sca1−Thy1.2high populations were compared using RNA-Seq of isolated telogenic (P56) dorsalskin. In line with results from the immunofluorescence assays, the expression of Thy1.2 was 100-fold higher in the CD34−Sca1−Thy1.2high population (FIG. 5K, 6P). Next, gene expression profiles of isolated cells were directly compared. As expected, the α6+CD34+Sca1−Thy− population was strongly enriched for HFSC bulge markers such as CD34, NFaTc1, TBX1, Lgr5, Lhx2 and K15 (FIG. 6P-Q). Consistent with the staining results, the α6+CD34−Thy1.2high population expressed low levels of these HFSC markers as well as barely detectable levels of the recently reported IFE SC marker, Dl×1. However, these cells expressed high levels of integrin α6 and β1, as well as a variety of keratins including K5, K14, K16 and K77, demonstrating that these cells are a subpopulation of basal keratinocytes (FIG. 6P-Q). Intriguingly, the levels of various intestinal SC signature genes such as Asc12, TNFRsf19, TNFsf10, FGFR4, Cdk6, Nid2, Elmo1 were also part of the Thy1.2high signature (FIG. 6P-Q). Furthermore, the expression of the known embryonic SC transcription factor Oct4 was ˜2.5 fold higher than in bulge CD34+HFSCs. Thus, Thy1.2 marks a transcriptionally distinct population of keratinocytes in the epidermis (FIG. 6P-S).

### Example 7: The Thy1.2 High SC Population Contributes to IFE Homeostasis

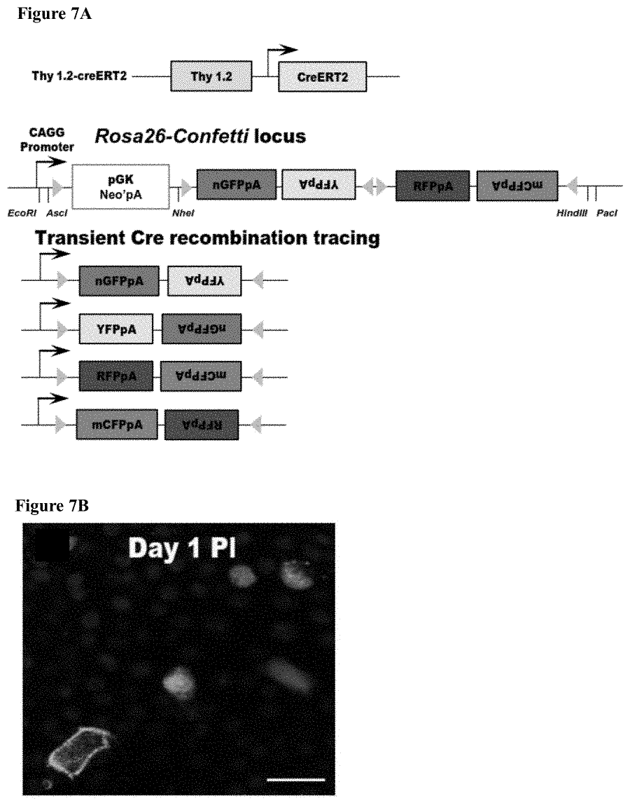

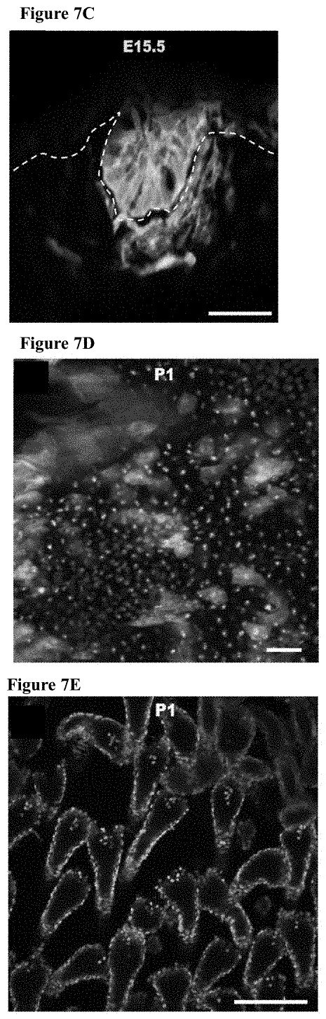

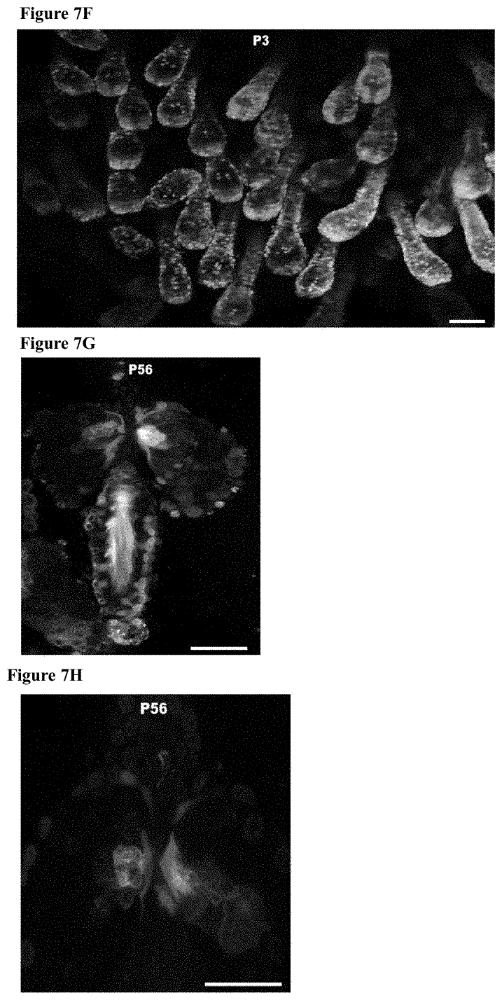

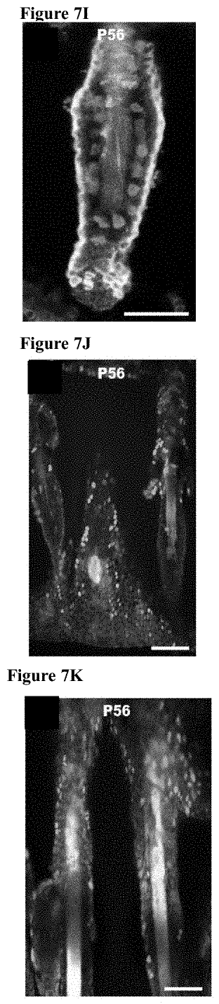

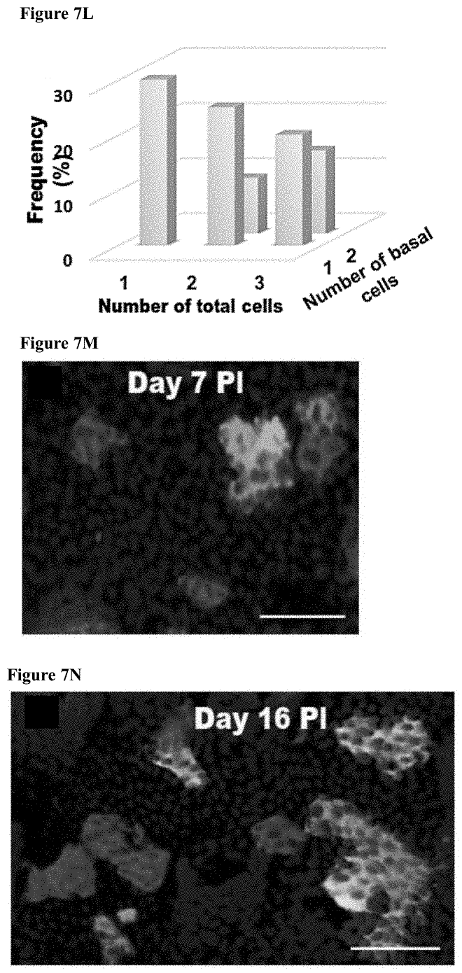

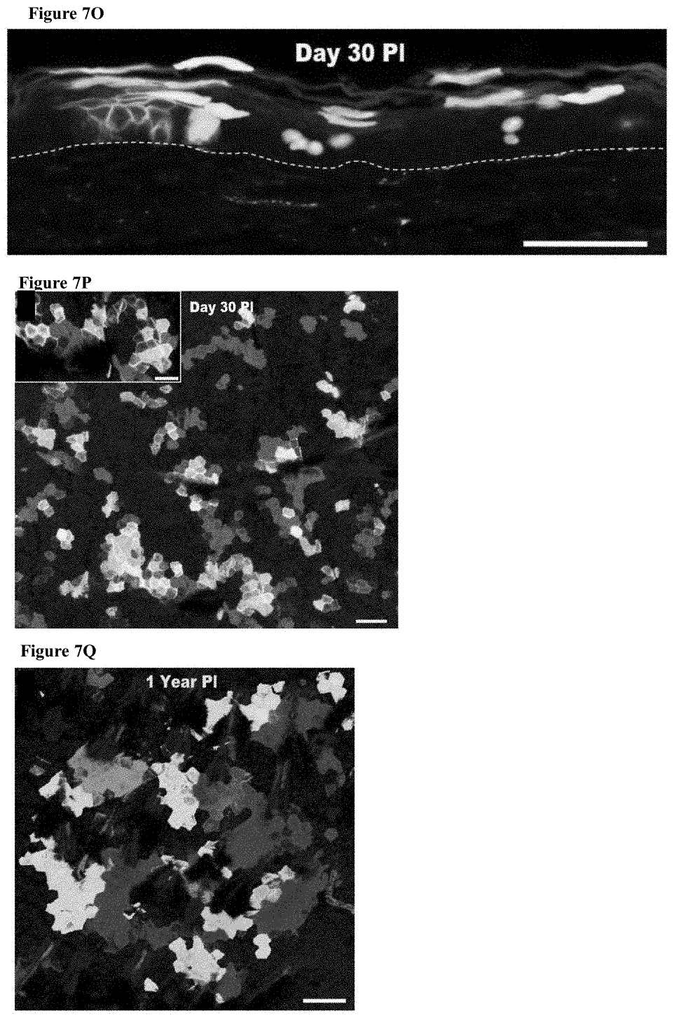

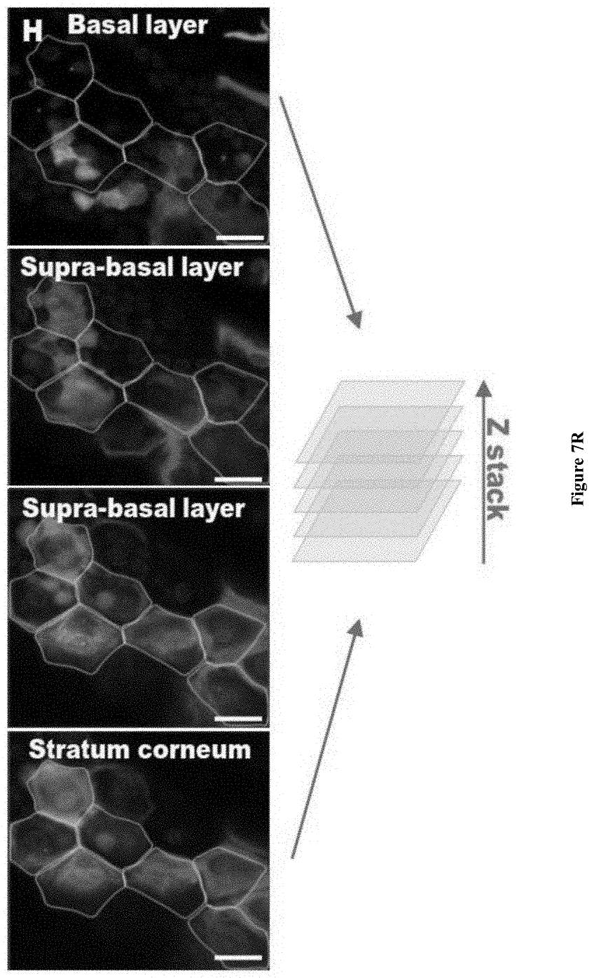

To examine the lineage potential of Thy1.2high cells, both the inducible Thy1.2CreERT2 and the constitutive Thy1.2-Cre mice were intercrossed with Rosa26-Confetti mice. In these mice, upon Cre activation and recombination, Thy1.2high cells randomly express one of four fluorescent proteins GFP, CFP, RFP and YFP, which are localized to different cellular compartments? (FIG. 7A-B). Consistent with the results from the expression studies, widespread labeling in the placodes (E15.5) and in the recruited fibroblasts and epidermis was observed (FIG. 7C). In both postnatal day 1 and 3 (P1 and P3), the HFs and epidermis were labeled in different clones (FIG. 7D-F). At P56 the HF bulge as well as the ORS and IRS, hair germ and SG were positively marked, while the base of the SG was also positively stained (FIG. 7G-I). Additionally, fluorescent clones were seen in the isthmus, IFD and IFE (FIG. 7J-K).

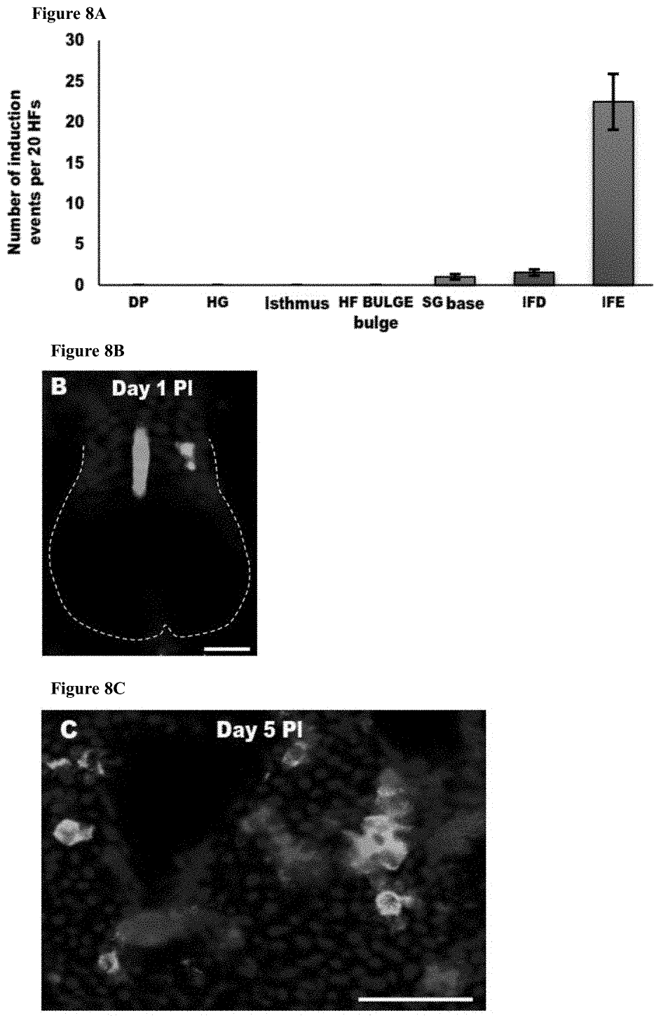

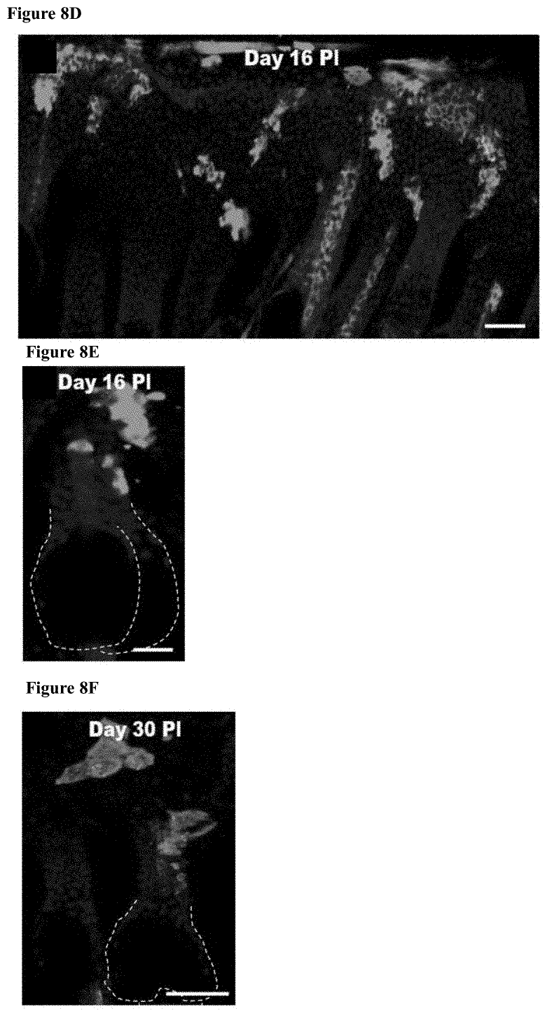

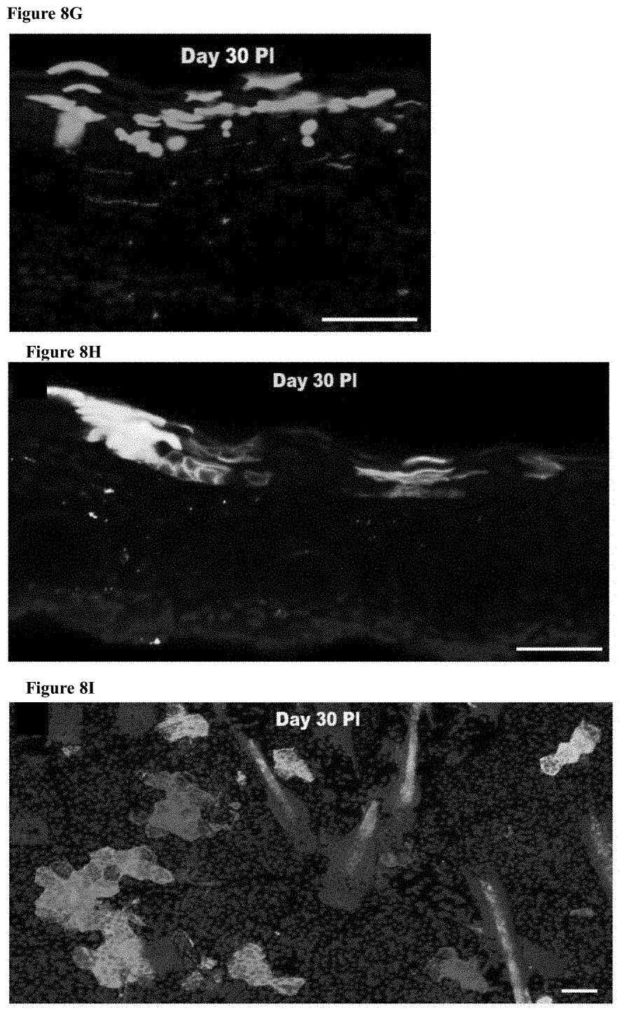

Next, lineage tracing was performed during the second telogen phase (P56) using the inducible Thy1.2CreERT2-Rosa26-Confetti mice. Induction was performed by either Tamoxifen injection or topical application. As early as 24 hours post-labeling, marked cells appeared at the basal layer of IFE, but never in the bulge, hair germ, dermal papillae or isthmus (FIG. 7B, 8A). However, occasional staining in the IFD and SG base was observed (FIG. 7B, 8A-B). All initial clones had a marked cell in the basal layer and −70% of labelled cells were located in the basal layer (FIG. 7L). When dorsal skins were harvested at 5, 7 and 16 days post induction (PI) it was clearly detected that labeled cells gave rise to clones of increasing size (FIG. 7M-N, 8C). Of note, in some instances trails spanning from the IFD to the IFE were also observed after 16 and 30 days PI (FIG. 8D-F).

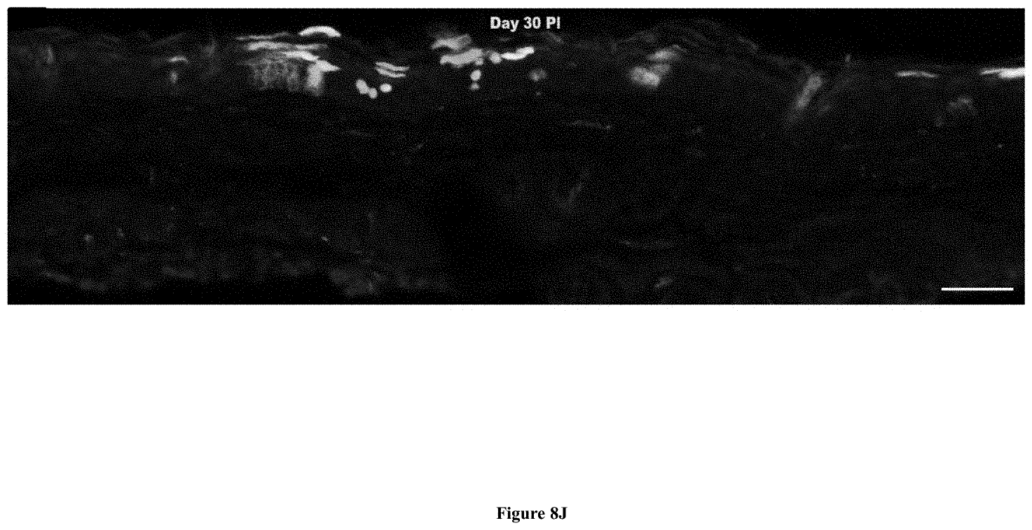

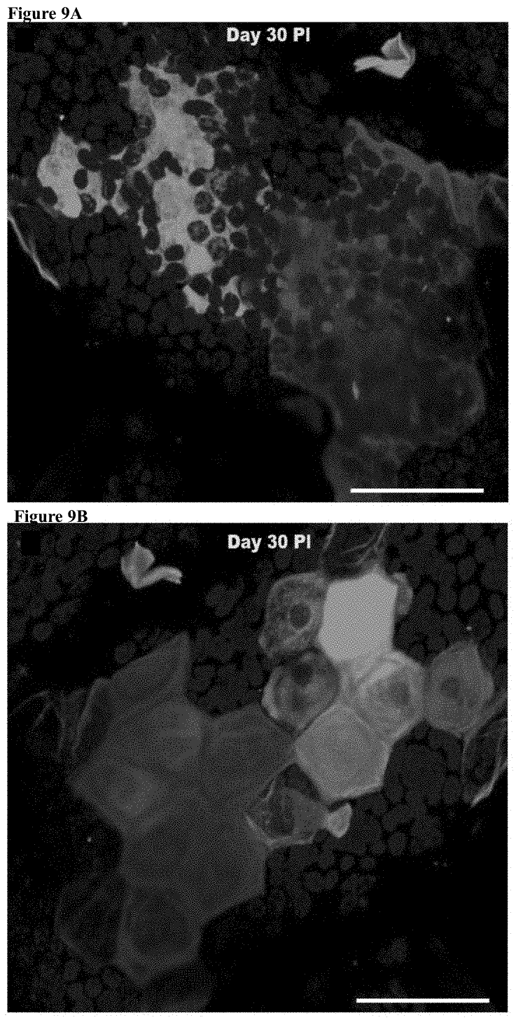

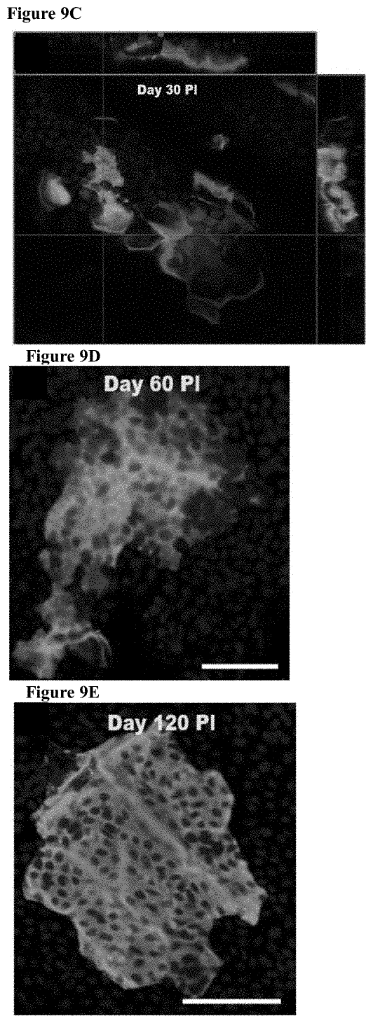

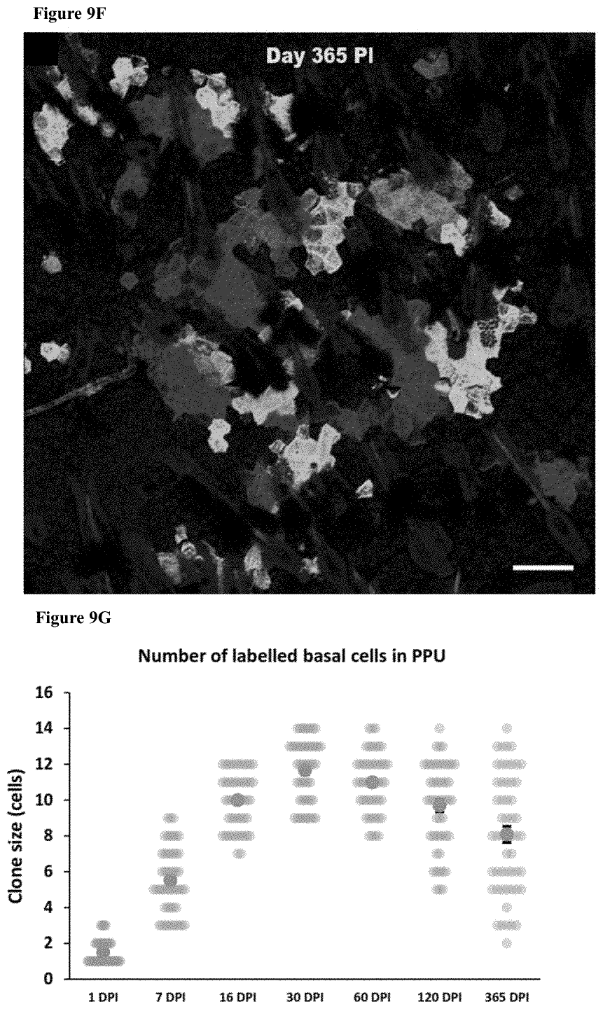

By day 30 PI, labeled cells emanating from the basal layer of the IFE were seen to differentiate into all layers, clearly indicating a crucial role for the Thy1.2+SCs in epidermal homeostasis (FIG. 70-Q, 8G-J). Importantly, the epidermis appeared as a mosaic of labeled clones where each individual clone displayed a polygonal shape (e.g. pentagon or hexagon), which was defined by the perimeter of the cornified layer (FIG. 7P-Q, 9A-C). These structures appeared similar to the units described by the EPU hypothesis, which predicts that each EPU harbors on average 10 cells in the basal layer with one slow cycling SC positioned in its center. These PPUs could clearly be detected at 60, 120 and 365 days PI (FIG. 7R, 9D-E, 9G), indicating that Thy1.2high SCs retain label for extended periods of time. Examination of the labeled clones 30, 60, 120 and 365 days PI indicated a footprint of 8-12 basal cells per PPU (FIG. 9F, G). Importantly, the PPU columns did not stretch from the basal to the cornified layer, cells could be detected outside the polygonal projections (FIG. 7S). However, the majority of labeled basal cells aligned vertically into the borders of the PPU (FIG. 7S). Additionally, in many scenarios the suprabasal cells appeared to cross the projected border, while cells of the granular layer were committed to the cornfield layers above (FIG. 7S). These finding are consistent with recent reports, which examined the spatiotemporal coordination in the mouse ear skin. These findings indicate that Thy1.2 marks a distinct label retaining SC population that highly contributes to IFE homeostasis.

### Example 8: Thy1.2 SCs Drive Wound Repair

It was next examined whether the distribution of Thy1.2+ cells correlates to individual PPUs. As expected, it could clearly be detected that Thy1.2+ cells become positively marked upon Tamoxifen induction and labeled PPUs retained a Thy1.2+SCs at their basal layer (FIG. 10A-C). Performing quantitative analysis of the spatial distribution of Thy1.2+ cells indicated that on average, the area per Thy1.2+ cell in the basal layer is 710±330 μm². While the average area per Thy1.2+ cell corresponds to the average PPU area of 717±150 μm², the variation in the distribution of Thy1.2+ cells in the basal layer was significantly larger than the variation in PPU sizes. The spatial organization of the Thy1.2+SCs in the basal layer was further analyzed by calculating the distribution of distances between neighboring Thy1.2+ cells and comparing it to a random Poisson process (see Example 9). Interestingly, it was found that Thy1.2+SCs are homogeneously distributed, yet with little geometric regularity in their arrangement (FIG. 10D-F). Taken together, these findings suggest that while Thy1.2+SCs in the basal layer are linked to the PPUs in the cornified layer, there is little support for the simplistic EPU model, in which clones are parallelogram-shaped structures that hypothetically would contain a single Thy1.2+SC in the center of their base.

Since distinct SCs are known to play a key role in wound healing, the contribution of Thy1.2+SCs to this process was examined. Lineage tracing followed by full-thickness excision wounds (1 cm²) was performed on the dorsal skin. Reporter expression was induced by Tamoxifen administration and wounding was executed on P51-old mice. Skins were analyzed at days 0, 5, 7, 18, 30, 60 and 120 post wound infliction (PWI). Significantly, Thy1.2high cells and their progeny contributed to wound repair, as evidenced by the presence of fluorescent clones migrating toward the center of the wound (FIG. 10G-Q). Intriguingly, labelled cells gave rise to a trail of fully developed PPUs spanning from the healthy skin towards the wound center (FIG. 10I-K, M-Q). However, careful examination of PPUs in the neo-epidermis (day 120 PWI) revealed that they displayed an abnormal organization of overlapping polygons (FIG. 10R-T). These results indicate that Thy1.2⁺ SCs generate trails of persisting clones along their migration path as part of their commitment to restore the wounded epidermis. Importantly, the data suggest that the process of generating and maintaining the scar tissue is achieved by Thy1.2+SCs that had migrated and facilitated the repair.

Collectively, these results identify a novel SC population, distinctly marked by Thy1.2, that play an integral role in fueling homeostasis and driving wound repair of the epidermis.

### Example 9: Testing the Order of the Spatial Distribution of Thy1.2⁺ Cells in the Basal Layer

The degree to which the spatial distribution of Thy1.2⁺ cells deviate from complete spatial randomness towards either clustering or regularity was investigated. The utilized approach conceptualized clustering, randomness and regularity as laying along a continuum. For a set of n points, where the distance between the points i and j is uᵢⱼ, the observed mean nearest neighbor distance is:

$$\frac{1}{n} {\sum }_{i = 1 ; i \neq j}^{n} min \{ {u}_{ij} \} .$$

Complete spatial randomness for n points in an area A is described by the Poisson process, in which the probability density function for nearest neighbor distance, d, is 2πδde^{-πδd}^{²} and δ=n/A is the point density, namely the mean number of points per unit area. The basic assumptions underlying this process are that a point is equally likely to fall at each location in the area, and that multiple points are chosen independently. The expectation value of this distribution gives the average distance between nearest neighbors for a random process.

The ratio of the observed and expected mean, R=r0/rE, gives the nearest neighbor statistics, which provides a quantitative summary of the spacing between the elements.

The average nearest neighbor distance decreases as points become more tightly clustered; hence, the closer to 0 the value for R becomes. The most tightly clustered situation is one where all points are superposed, when the value of R equals 0. The closer the points are to being randomly scattered, the more similar are the values of r0 and rE and the closer to 1 the value for R becomes. The value of R equals 2.149 for points that are spaced with perfect uniformity, as in a triangular lattice arrangement. Hence, the closer to 2.149 the value for R becomes, the more uniformly spaced are the points.

The basal layer figures of 5 independent sessions were analyzed to determine whether the distribution of Thy cells does exhibit some regularity. The resulting nearest neighbor statistics correspond to 1.25≤R≤1.41.

These results indicate that the spatial distribution of Thy cells in the basal layer is significantly closer to spatial randomness than regularity.

### Example 10: Average Area Per Thy1.2⁺ Cell in the Basal Membrane & Area Per PPU

To find the average area per Thy1.2⁺ cell, A_{Thy}, and the resulting Thy1.2⁺ cell density per polygon, the Voronoi diagram for the cell centroids was used. The Voronoi diagram is the partitioning of the figure plane into regions containing all points closer to a given centroid than any other.

The Voronoi region for the kth centroid is defined as: Rₖ={x∈X|d(x, Cₖ)≤d(x, Cⱼ)}, for all j≠k. A_{Thy} as is defined as the average Voronoi region area for each basal layer figure. Averaging over all figures we obtain A_{Thy}=710±329 μm².

The area per PPU was estimated by direct measurement of PPU area in the apical layer and found to be A_{Pol.}=717±149 μm².

### Example 11: Mixed Sebaceous Gland Organoid

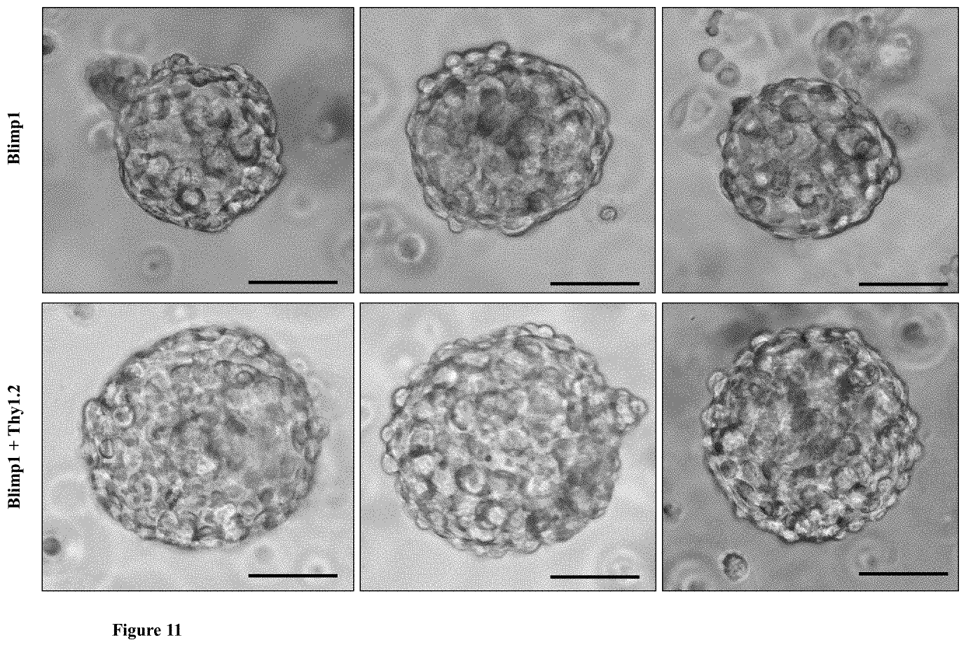

In order to improve on the sebaceous gland organoid derived from only Blimp1 positive cells, Blimp1⁺ cells were cultured alone or with Thy1.2+SCs in a ratio of 1:1. The morphology of the resulting organoids was monitored for 14 days. SB organoids derived from the coculture shared a similar morphology to the one described herein for organoids from only Blimp1⁺ cells. Further, the organoids derived from the mixture of cells were significantly larger, with a larger average diameter and a greater number of cells in the interior (FIG. 11). Thus, the coculture of the two stem cells provides an avenue for generating model sebaceous glands that may more closely mimic an endogenous organ.

### Example 12: The Sebaceous Gland Organoid can be Used as a Model Organ to Test Drugs

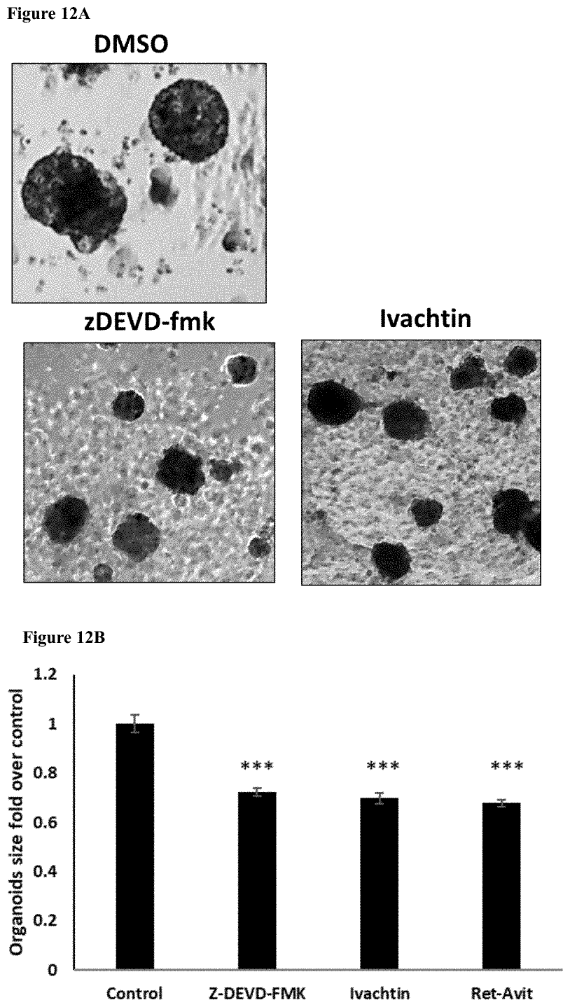

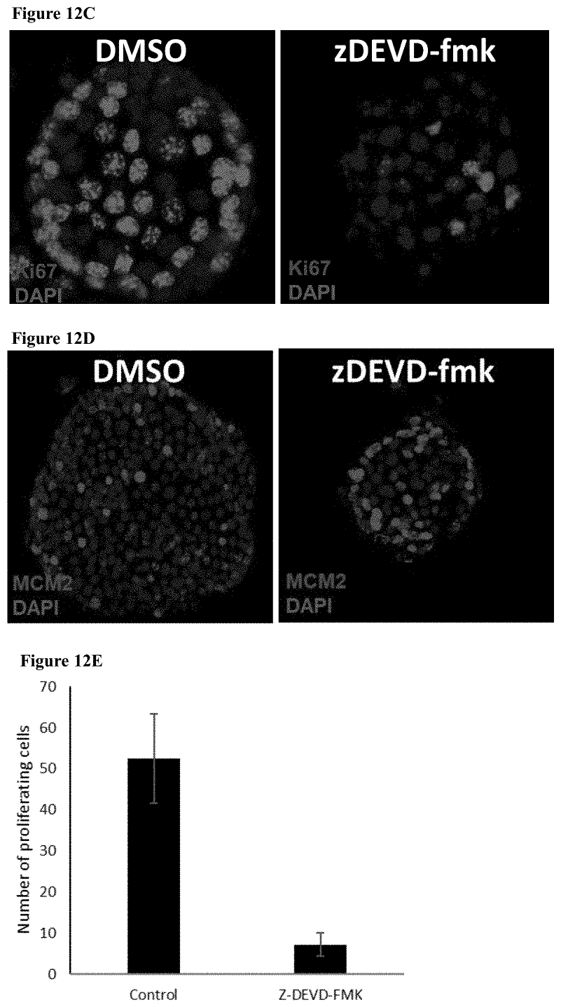

As the sebaceous gland organoid closely mimics an in vivo SG, it was next tested whether it could be used as a model system for drug evaluation and discovery. Organoids were generated as described herein and treated with two caspase-3 inhibitors (zDEVD-fmk at 100 uM and Ivachtin at 100 uM) to assess their effect on the SG. A known anti-acne drug, Tretinoin (10-100 uM) was also added as a positive control. After 72 hours both caspase-3 inhibitors attenuated SG organoid size by about 30% (FIG. 12A). This effect was very similar to the reduction caused by Tretinoin (FIG. 12B). It is likely that the size reduction was the result of a decrease in cell proliferation, as cells positive for Ki67 and MCM2 were greatly reduced by treatment with the caspase-3 inhibitor (FIG. 12C-E). This experiment confirms that the SG organoid can be used as a model system for the in vivo SQ.

### Example 13: SG Organoids can Serve as a Model for Acne Vulgaris

It was further examined whether this platform could be beneficial for the study of SG pathologies. Since Acne vulgaris effects millions of lives worldwide, conditions were generated where the SG organoids potentially exhibit aspects of this pathology. Acne vulgaris is characterized by an increase in SG size and is dependent upon sebocyte hyperproliferation and sebum production that serves as a nutrient source for Propionibacterium acnes. Additionally, androgen stimulation has been found to play a critical role in regulating sebocyte proliferation and driving the emergence of acne, while PPARs have been shown to alter sebaceous lipid production and modulate acne formation.

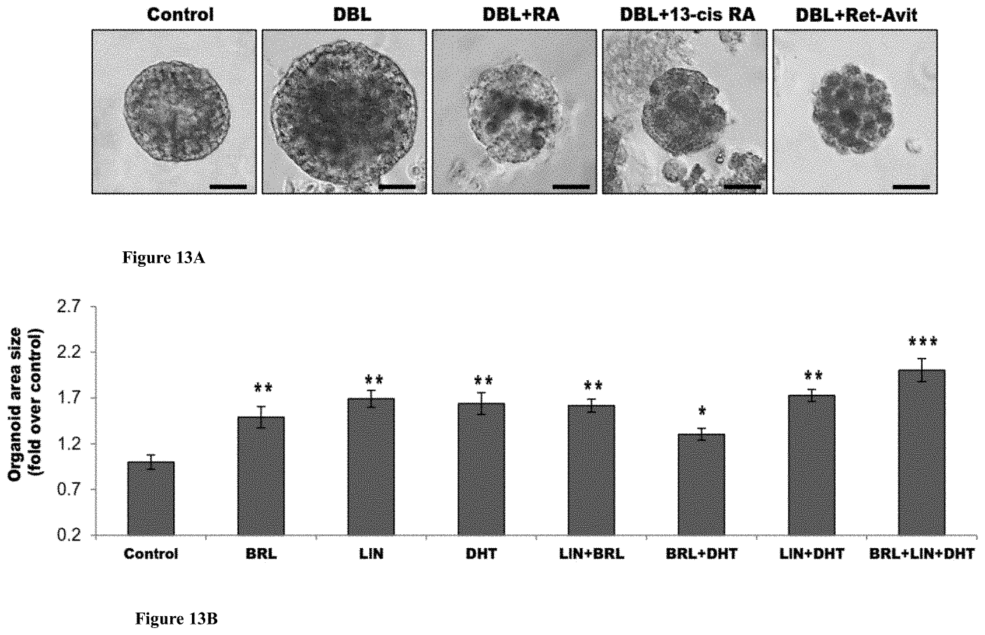

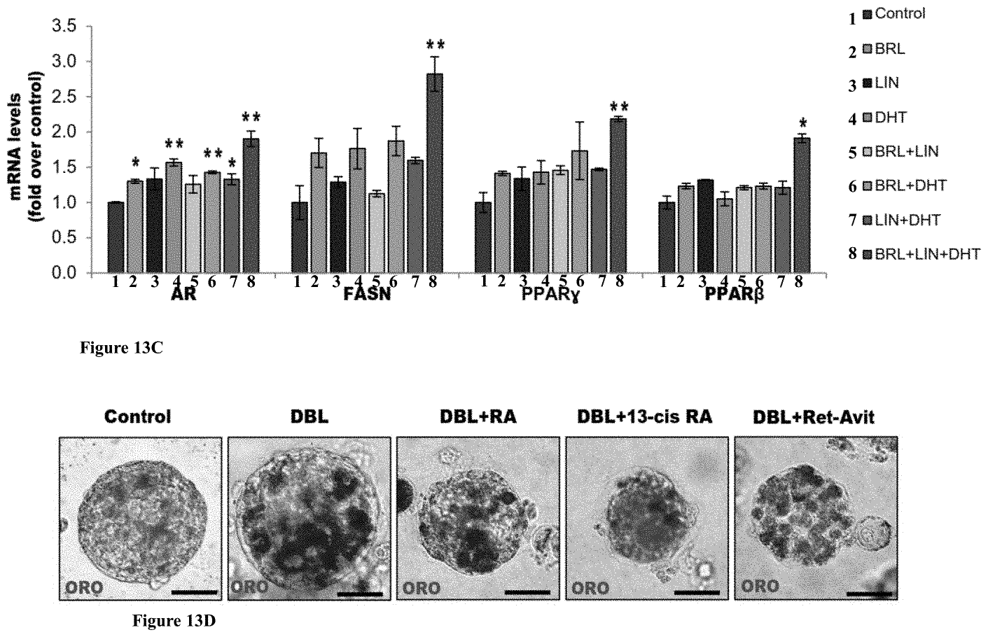

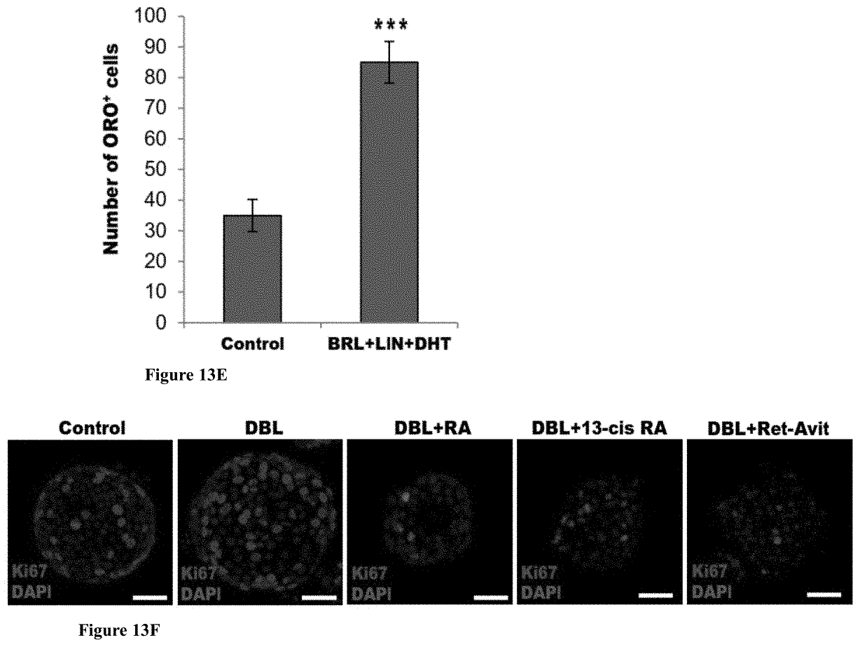

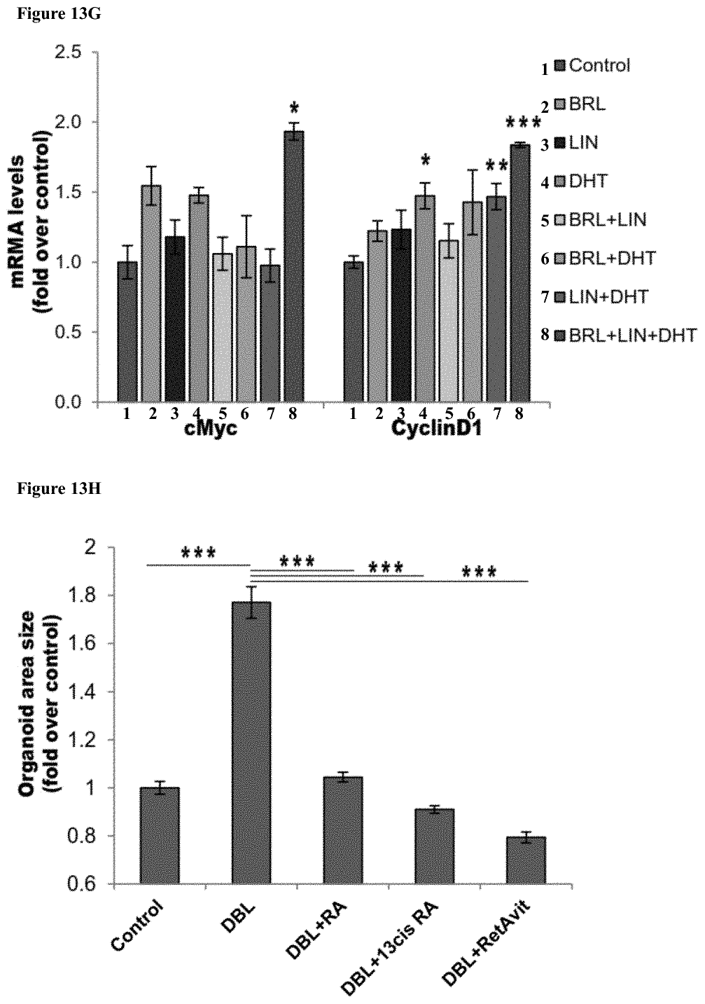

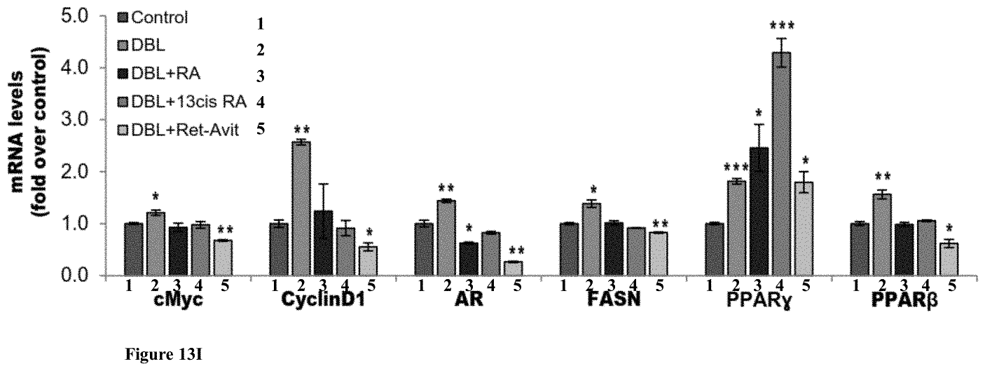

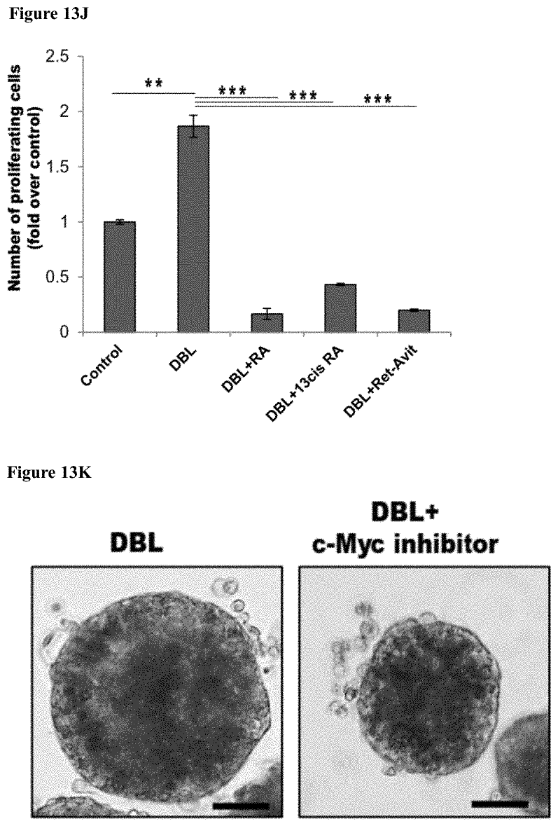

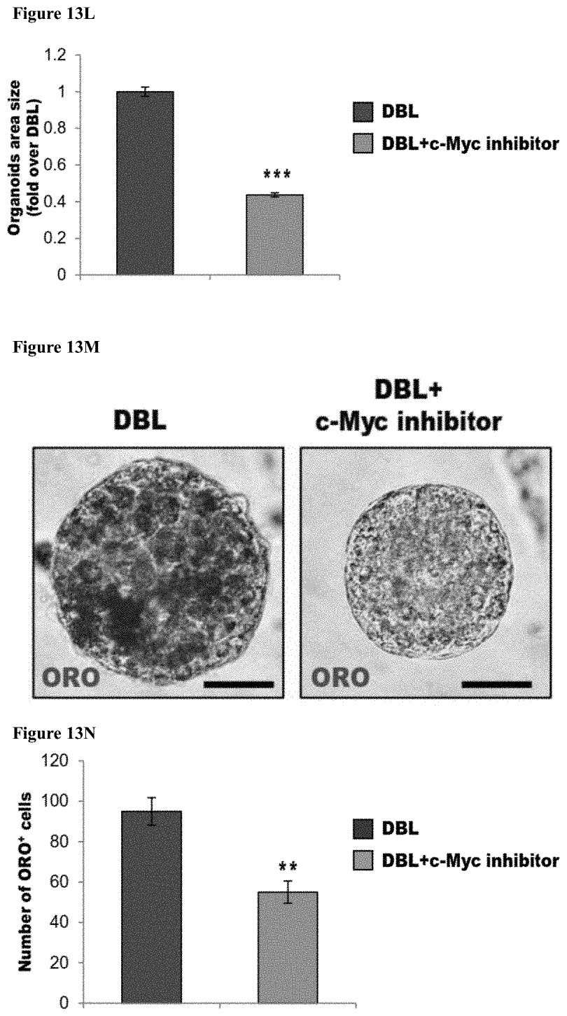

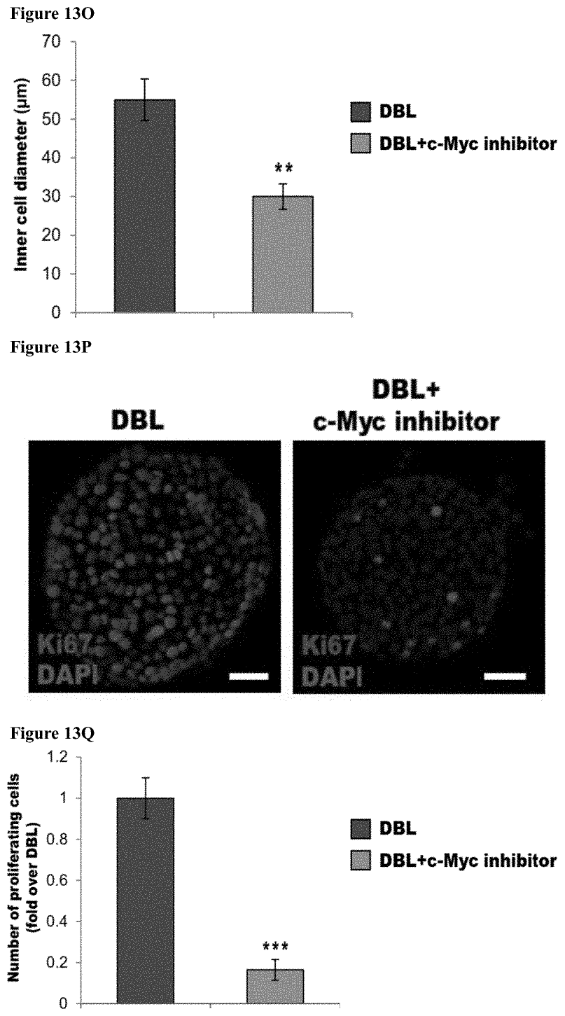

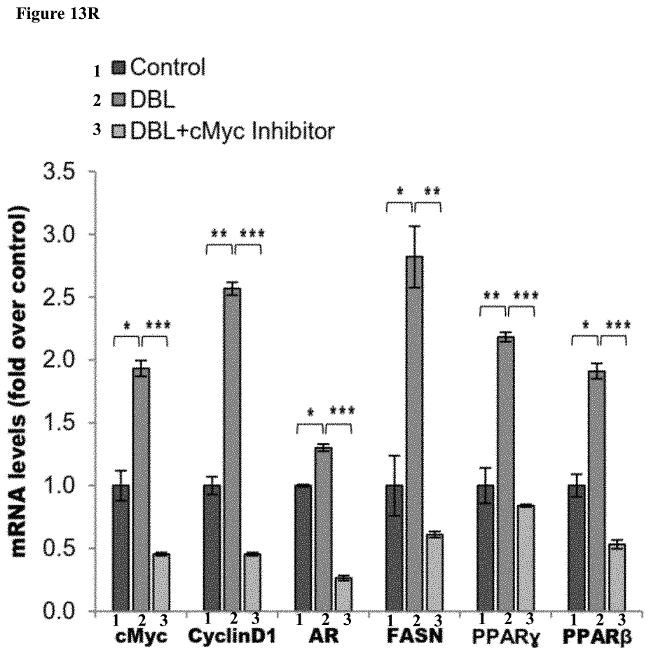

Therefore, as a first step, the potent dihydrotestosterone (DHT) androgen, the PPAR-γ BRL-49653 (BRL) activator and linoleic acid (LIN) known to activate PPAR-B38 were administered to organoids. Administration of BRL, LIN or DHT for 7 days significantly increased the size of individual SG organoids. While dual combinations did not have an additive effect on organoid size, the combined administration of DHT, BRL and LIN (denoted DBL) resulted in significantly larger organoids (FIG. 13A-B). In accordance, treatment with DBL led to the most considerable increase in mRNA levels of AR, FASN, PPAR-B and PPAR-γ, suggestive of increased lipid synthesis (FIG. 13C).

In order to assess whether sebum production was indeed affected in response to DBL treatment, ORO staining was performed and a significant increase in the number of ORO+ cells was observed (FIG. 13D-E). Of note, as in control organoids and normal SGs, all ORO+ cells were located in the inner differentiated part of the DBL-treated organoids (FIG. 13D). To examine whether proliferation was also affected staining against Ki67 was performed and a significant increase in the numbers of Ki67+ cells upon DBL treatment was detected (FIG. 13F). These results were verified by RT-PCR analysis, indicating the most significant increase in levels of c-Myc and Cyclin D1 transcripts upon DBL treatment (FIG. 13G).

With a system that recapitulates important aspects of acne vulgaris, the extent that currently prescribed medications could affect its development was examined. Retinoids are derivatives of vitamin A, which serve as the first-line treatment for acne. Specifically, all-trans-Retinoic acid (ATRA; Tretinoin), formulated ATRA (Ret-Avit) and 13-cis RA (Isotrtinoin) are used worldwide. Administration of all three retinoids to DBL-treated organoids resulted in a significant decrease in organoid size with formulated ATRA yielding the most robust results (FIG. 13D, 13H). Furthermore, retinoid administration instructed cell differentiation leading to a heightened number of ORO+ cells (FIG. 13D). Interestingly, in these conditions ORO+ cells were also detected along the SGPZ, suggestive of an altered differentiation program.

The differentiation program in the SG is a multi-step process. Cells prior to the onset or during early stage of differentiation express AR, which is known to reside upstream and lead to the expression of FASN35. In response to the different retinol treatments, a significant decrease in the mRNA levels of AR and FASN was detected, indicating that early differentiating cells were not abundant in these organoids (FIG. 13I). Additionally, the mRNA level of PPAR-β was also significantly decreased (FIG. 13I). Interestingly, the level of PPAR-γ, which is known to be expressed during middle/late differentiation, was significantly increased (FIG. 13I). This result, in combination with the high numbers and location of ORO+ cells, suggests that retinol treatments can advance and alter the differentiation process. In addition to increased differentiation, it was found that all retinol treatments led to a drastic decrease in proliferation levels as indicated by Ki67 staining (FIG. 13F, 13J) and RT-PCR analysis examining the mRNA levels of Cyclin D1 and c-Myc.

Retinols, such as 13-cis RA, are known to have severe side-effects and concerns have been raised regarding the teratogenicity of these compounds. This highlights the immediate need for alternative treatments. Interestingly, a recent genome wide association study of severe teenage acne has suggested c-Myc as a potential regulator. Therefore, it was hypothesized that inhibition of c-Myc could serve as a beneficial treatment of acne vulgaris. For this aim, the 10058-F4 c-Myc inhibitor was administered to DBL-treated organoids and size, proliferation and differentiation were monitored. The results clearly show that c-Myc inhibition results in a significant decrease in organoid size similar to that of Retinol treatment (FIG. 13K-L). Interestingly, c-Myc inhibition led to a significant decrease in ORO+ cells (FIG. 13M-N). Moreover, a decrease in the size of individual inner cells (FIG. 130) and significantly lower expression levels of lipid synthesis genes, indicative of diminished differentiation was observed (FIG. 13P). Finally, cellular proliferation was monitored and a more than 80% decrease in the number of Ki67+ cells was observed (FIG. 13P-Q), correlating to a significant decrease in the levels of Cyclin D1 and c-Myc transcripts (FIG. 13R).

Taken together, these results indicate a potential role of c-Myc in the pathogenesis of acne vulgaris and suggest that it may be utilized as a therapeutic target.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A sebaceous gland (SG)-like organoid, comprising:
a. an outer layer of cells, wherein said outer layer comprises a proliferating cell, a stem cell and a B-lymphocyte-induced nuclear maturation protein 1 (Blimp1) expressing cell; and
b. an inner core of non-proliferating cells wherein said inner core comprise a Blimp1 expressing cell.

2. The SG-like organoid of claim 1, wherein said outer layer stem cell expresses Blimp1 or wherein every stem cell of said outer layer expresses Blimp 1.

3. The SG-like organoid of claim 1, wherein said outer layer comprises cells expressing Keratin 5 (K-5), Keratin 15 (K-15), c-myc or a combination thereof.

4. The SG-like organoid of claim 1, wherein at least one outer layer proliferating cell does not express Blimp 1.

5. (canceled)

6. The SG-like organoid of claim 1, wherein said organoid is derived from a Blimp1 expressing stem cell.

7. The SG-like organoid of claim 1, wherein said organoid is devoid of immortalized cells, genetically modified cells, or both.

8. The SG-like organoid of claim 1, wherein said organoid is characterized by outer layer cells that migrate to said inner cell core, a portion of said outer layer cells that divide asymmetrically into an outer layer cell and an inner core cell or both.

9. (canceled)

10. The SG-like organoid of claim 1, wherein said inner core
a. comprises at least one of: cells that are larger than said outer layer cells, sebocyte-like cells, and lipid-producing cells;
b. is devoid of proliferative cells; or
c. both.

11. (canceled)

12. (canceled)

13. The SG-like organoid of claim 1, wherein said inner core Blimp1 expressing cells are terminally differentiated.

14. A method of producing a SG-like organoid of claim 1, the method comprising culturing a Blimp1 expressing skin stem cell in a 3D growth matrix for a time sufficient for the production of said SG-like organoid.

15. The method of claim 14, wherein said Blimp1 expressing skin stem cell is a primary epidermal keratinocyte stem cell.

16. (canceled)

17. The method of claim 15, wherein said epidermal keratinocyte expresses integrin alpha 6 (α6), does not express lymphocyte antigen 6 complex (SCA-1), or both; or further comprising preculturing said primary epidermal keratinocyte stem cell on a feeder layer before said culturing in a 3D growth matrix.

18. The method of claim 14, wherein only Blimp1 expressing skin cells are cultured in said 3D growth matrix.

19. The method of claim 14, further comprising at least one of:
a. coculturing a Thy1.2 expressing stem cell in said 3D growth matrix with said Blimp1 expressing skin cell; and
b. preculturing said Blimp1 expressing skin stem cell on feeder cells for at least 4 passages before transferring at least one Blimp1 expressing skin stem cell to said 3D growth matrix.

20. The method of claim 14, wherein said culturing comprises at least one of:
a. culturing in Matrigel;
b. addition of media, and wherein said media is hair follicle stem cell media;
c. culturing in media comprising 45-55 mM Ca2+;
d. culturing in media comprising at least one growth factor selected from epidermal growth factor (EGF), fibroblast growth factor (FGF), Noggin and R-spondin;
e. culturing is in media comprising a ROCK inhibitor; and
f. culturing in the presence of an agent that inhibits cell-cell interaction-dependent anoikis.

21. (canceled)

22. (canceled)

23. The method of claim 14, wherein said time sufficient for the production of said SG-like organoid is at least 8 days.

24. A method of ex vivo testing a skin drug, the method comprising contacting a SG-like organoid of claim 1 with said skin drug.

25. The method of claim 24, wherein said skin drug is for treating acne, further comprising contacting said SG-like organoid with at least one of dihydrotestosterone androgen (DHT), PPAR-γ BRL-49653 activator (BRL) and linoleic acid (LIN) before contacting with said skin drug, or both.

26. (canceled)

27. A method of treating acne in a subject in need thereof, the method comprising decreasing c-Myc expression, function or both in a sebaceous gland of said subject, thereby treating acne in a subject.

28. The method of claim 27, wherein said decreasing c-Myc function comprises contacting said sebaceous gland with a pharmaceutical composition comprising a c-Myc inhibitor, optionally wherein said c-Myc inhibitor is 10058-F4, or wherein said treating comprises at least one of decreasing the size of said SG; decreasing lipid production by said SG; and decreasing transcription of PPAR-γ, Cyclin D or both.

29. (canceled)

30. (canceled)


SEQUENCE LISTING (10 sequences)

SEQ ID NO: 1 - DNA, 20 bp, Mus musculus
gcgacctggaagtccaacta

SEQ ID NO: 2 - DNA, 19 bp, Mus musculus
atctgcttggagcccacat

SEQ ID NO: 3 - DNA, 21 bp, Mus musculus
agaatcccacatcctgctcaa

SEQ ID NO: 4 - DNA, 21 bp, Mus musculus
aagtccacgctcaccatatgg

SEQ ID NO: 5 - DNA, 20 bp, Mus musculus
gatggaagaccactcgcatt

SEQ ID NO: 6 - DNA, 20 bp, Mus musculus
aaccattgggtcagctcttg

SEQ ID NO: 7 - DNA, 18 bp, Mus musculus
ttccttccagcagctgtg

SEQ ID NO: 8 - DNA, 15 bp, Mus musculus
tcgcacgcgtggacc

SEQ ID NO: 9 - DNA, 20 bp, Mus musculus
cccttgatgaagagggatca

SEQ ID NO: 10 - DNA, 20 bp, Mus musculus
actccacaggtgggaacaag